SEQUENCE LISTING (106 sequences)

SEQ ID NO: 1 - DNA, 1269 bp, Agrobacterium tumefaciens
  CDS (121)..(1146)
cagccggcgcaggtcggcggatagacgaggcggaagaacgaacgcgccaccgcgcgtgccgcggaaggcacgacggcggacttcgacagcaatcccgaaaacagatcgtcaccagcactcatgaggttgactttagcgccgaacgggtgcctttgcgagagcgatacgctccggcgcgacaccggaaataatgcccgaagcggcatgacagcaacgataaggcccgccgacccagaaggcgttgaaggccgagcaggcaagacgatggatattctctttgaccaggccctgatcgaacaaaaccgccgccgtgcgtggacccggcgtgatgacaaggccctgtttcttctcgacatggcagccgaagagctggccgacaggcttgccatcgtcgagcgcaagttcgagacggccatcgaactgcacggcggaacgggcgtgacggcccgccggctggccgagacgggaaaagtggacaacattcgccgcatcgaaaccgaaagcgcattcgcgacggacggaaacattcccgaaacggcgtcgatggagcatctgccgctcgcggaagcctccgccaatctcatcgtttcaccgcttgccctgcatctcaccaacgacacccccggtgcgctgatccagatacggcgcgcattgaagcccgatggtctgtttctcggcgccattcccggcagcggcaccctgcatgagctgcgcgatgtcttgctgacagcagaagcggagctgaccggaggtgccagcccgcgcgtcattcccttcgccgacgtgcgcgatgtcggtgcgctcctgcaacgcgccggttttgctcttcccgtcaccgacgcggagacctacacggtgcgttatgattcgattttcccgctgatgcgcgacctgcgcgccatgggcatggccaacccgctggcaagcagaagccgaaagccgctcaaccgcgcctttttcctgcgggcggcggaactctatgccgagcgctattcggatcccgacgggcggataagggcgacattttccatcatctatgtatcaggctgggcgccgcatgagagccagcaaaagcccctgaaacccggctccgccaagatgcgccttgctgacgccttgaagacaacagaagtaaaactgtcctgaatgatcggcgcgagctttcaacccttgggaaaagcgtcgacgatgaactgaaacacggcattgataccgctgctgatgttgccgaggccggcgatgagcgctgcggaaattatccctgcgatc

SEQ ID NO: 2 - DNA, 687 bp, Agrobacterium tumefaciens
  CDS (121)..(564)
gatatcgatgcggcgaaggctctcgccgatgccgatccctatgccaaggccggcctgttcgagagcgtggatgtgaagccctataactgggtcttcaacaatccgggagcgtgaccggagatggcgaattattggctgtacaagtccgagccgttcaagtggtcctgggaaatgcagaaggccaagggcgagaccggcgaagaatggaccggcgtgcgcaactatcaggcccgcaacaacatgcgcgccatgaagatcggcgacaagggtttcttctatcactccaatgaggggctggatgtcgtcggtatcgtcgaagtctgtgccttgtcgcatccggattcgactgcggagggtgatctgaagtgggattgcgttgatatccgcgccgtctgcgatatgccgcagccggtttccctgaaggatgtgaaggcgaacccaaagctcgaaaagatgtcgctcgtcacctcgatgcggctttccgtgcagccggtgacggaagaggaatatcttgaggtctgccgcatgggcggcctcgccaatccgccgaaatcgccggactgatgcttcggtgaggaccgatcccgagcgcttcattctcgacaatgctgccatcatgcatccgccgcatgtgccggaattgcgtcttcatctcgccaccgaggcgcacgagctttggctgaagac

SEQ ID NO: 3 - DNA, 942 bp, Agrobacterium tumefaciens
  CDS (121)..(819)
cccgcagcgttccctccaaggcgaagctcaacaggatgatgatgaaagtgcgtgagctgggttatgatgtcaaaaagctcgaatttcccgcccagaaataaccgctgacggagagcgccgatgaagacccttgaaaacggctatcgagccatcgtcatcggtgcgtccggcggtattggcgctgcattgcttgcgcgcctcaaggccgatccccggtgtgcatttgccctgcctctgtctcgcagccacgacggtctcgacatcaccgacgagacgacggtccgcgaagccgctgcccgtctggaagaccagcgcggttcattcgatctgatcttcaacgcaacgggtgctttggtggtcgatggtgccggtccagaaaagtcgatacgggcgatcgacgccgatctcatggcgaaacaatttgcggtcaatgccatagggcctgcactgctcctgaaatatttcacgccgcttttgcaacgggacaggcgaagcgtctttgcctccctgtcggcgcgtgttggctcgattggcgataaccggctgggcgggtggatttcctatcgcgccgcgaaggccgctcaaaatcagatcattcgaactgcggcgatcgaaattgctcgcaccaaccgccaggccatcatcgtcgcgctgcatcccgggacagtcgatacggacctttcccagccgttctcgaagggcagagaccggttcactccggatcactcggctgtgcgccttttggagaccatcaacggtctcgacgagagccagacgggccgatttttcgcccatgacggctcaacgatagaatggtgagttctgacttatacagtgctgttgcgagcaacagcaactcgacgaaacgagctataagttttcaacatagcgactttgcgcattgcagtctgatgaactcggccttctcggcgttcctgacct

SEQ ID NO: 4 - DNA, 1095 bp, Agrobacterium tumefaciens
  CDS (121)..(972)
ccaagtaaggcttggcgaacggaatttttcgaacacccggccttcgtgccgggtgttctgcttttggcatgtcgcgcggtgtcacaagcctgtcatcagccgggtttatcgcggcgcatcatgactgaaaaaagcacacccctcttctccttcggcattgtcgccgatccccaatatgccgacgccgatccccgcccggatatgggccgttattatgccgaaagcccgaaaaagctggctgaggcgatcaatgtcttcaacagcgaggacctcgccttcgtggtgacgctcggcgatatcatcgatcgcggctttgaaaatttcgatgcgatcctcagcgtttatgacgcgcttcgccacccctcggtgctgctgccgggcaatcacgatttcgccgtcgcccccgagcatctttccgccatccacgcccgcctcggcatgaaggccccctggcatgattttgccatgagcggcatccgcttcgtcgtgcttgacggcaacgatgtcagcctcttcgccccgccacctgatgatccgcgccgcctggtcgcgcaggagcggttgaagcgcctctgggatgtcgatgcgatcaatgcgcaggactggaacgcctcactcagtgacaagcaattcgaatggttgcgcaccattctgcaacaggcggatgcggccggcgaaagagtaatcgtgctgtgccactatcccgtctatccggaaaacgcccacaacatgtgggatgcgccgcgcatccttgatcttctcggttctcactcctccgttgccgcatggttcaacggccataatcacgagggtaattacgggacgctcggcaccacccacttcgtcaatttcaagggcatggtggatacgcccgaccagaacacctttgccatcgccgatgtcttcgccgatcggatcgcgattcgcggttttggacgcgaggaggatcgggtgttgcagtacttacggtaaagtgacgcctgagatcggccgccccaactctccacccggctggccgctctgccagccgctcatccacaatcgccgcagcttctcgccttccatcttgaagaagctgcccgtcgcggcacaatc

SEQ ID NO: 5 - DNA, 1209 bp, Agrobacterium tumefaciens
  CDS (121)..(1086)
cggaaaaatttcacggcctatggttaccagcgttccatgacagaaaatttccacgcgctgacgaggtgcgggcaaaccgctctttgcgggcagtcttcgttttcggcatagcggggttttatggtcggatggtggccggtaggccgcaggcaggcctggcaccgcttttttccttgccatcggcgtcccgctttgcgatgtgacgcacactcttttttcgcacccggaaagcacatcatggcccatcgcattctcaccctcggcgaaatcactgatgggttcgacgttgttctttcggatgtctggggcgtgctgcacaatggcgtcagcgcctttccggatgcggctgttgcgctgcatgaagcgcgcaaggcgggcaagacggtggtgctcatcaccaattcgccgcgcccggcccccggcgtcatcgcccagctgcgtgttctcggcgtgcctgacgaggcctatgaccgtatcatcacctcaggtgatgtcacccgcggcctgatcgccgaaggcccgaaaaaagtcttcctgctcggtcccgagcgggacatgccgctgctggaaggtctcgatgtcgagcgggtcggcgaggctgaggcgcaatccgtggtctgcaccggcttctttgatgacgagacggaaacgcccgaagattatacggaaatgctgaagggcttcatcgcccgcaaggtgccgatgatctgcgccaaccccgacctggtggtggagcgcggcgagcgcatcatcccctgcgccggcgcaatggcggcctattacgaacagctgggcggcgaagtccgcattgccggcaagccgcatgcgccgatctatgaagcctgccttgcggcggccaaagacgtgcgcggcgccttcgccaaggatcgtgtgctggccatcggcgacggcatgccgacggatgtgaagggtgcgatcgcgagtggcctcaaccttctttatatcagcggcggcattcacgccgctgaatatacgctgaacggccagacggacgaggcgcttctgaatgcctatctgaaggggcagggtgctgctcccggctggtggatgccccgtcttgcctaagaaagtcttgcctaagaacgtctcgcctaagctggcgtaaaaattggatcgactgccatgaccgtctttcatcgcaatgaaaaaaaagagccgttgccggaagctcttcgcggtggcgttatc

SEQ ID NO: 6 - DNA, 1335 bp, Agrobacterium tumefaciens
  CDS (121)..(1212)
acaggaggcagcgccacggacaatagcaaaattttgagcgcaaaccacgattccccaccgtcacagaccctgacatgatcctgtaacaagcgcgccgcatatacgggcagaagccgggcaatggggattcgcccgagcgcagatgagcaggatcagtgtttcatgacgagaatcacgattgtcacagatgcctggcacccgcaggtcaacggcgtggttcgctcgatcgaaaacaccaataccgaactggcgcgactgggcgtggatgtgcgcatggtcacgccgcagagtttttacagcatcccctgccccacctatccggaaatccgcctgtcggtcgccggttatcgccgcgtggcggtagagattgaaaaaagccagccctccttcgtgcacatagccacggaagggccgctgggcttcatggcccggcgctggtgcgtgaagaaccatatgcggttttccaccagctatcacacccgctttcccgaatatgtcgcggcacgtttccctgttccggaaagctggctttatgctttcgtgcgctggttccacaatggcggcaacacctgcatggtggctaccccgagcctcgagaccgaactggaagcgcgcggcgttcgcaacctcaagcgctggagccgcggcatcgacgcggagcttttccatccccgcacgaaaacaaagctgcccttcgacctgccgagaccgatcttcatgacggtgggccgggtggcggtggaaaagaacctgccggaattcctggacctcgatctgccgggatcgaagatcgtcatcggcgacggccccgcccgctatgaattgcaggaaaaataccctgatgttctgttcaccggcatcaagaccggtgaggagctggccgatgcctatgcccaggccgatgtcttcgttttcccgtcgaagaccgacacattcggcaacaccattctggaagcgctggccagcggagtgcccgtggccgccttccccgtcaccgggccgatcgacattctcggcggcaaccccgccgccggcgcactggacaataacctgcgcgatgcctgtctggcagcgcttcactgctcaccacaggcagcgcttgcgctttccaaaagctacagctgggaaaaggcctcgcggcaattccttgacaacgtcatccacgcagccggaaaatcgctgccgcttctgtcgcgctcgcggcttgcatgaggcaatccccgagatttgtgcgggaatgagtagacgaaagggtgcgggagtgtcacaaggcgatcaggacgcgactgccgttatctctagaacggggtcgggctgctgattgacgtcaggcgg

SEQ ID NO: 7 - DNA, 801 bp, Agrobacterium tumefaciens
  CDS (121)..(678)
gacacaacgcgttcactccgggtgggggcgtgccggcatgatgaatttatcggctgacaaagaccggaacctgaactagacataatatattagacggcgatactcacaacgtggatcgcaatgctactgcgctggtctctcatcgtcctgctgactttcggactttccctcgattccgccgctgcgacggaaaccgacgcggtgggcgccttccaccacgccgttcaggtgggcgatgtccaaaccattcgaaccatgattgcggctgaccccggtttggccacgtcaagggacaagttcggtttccagccgatccatcttctcgacatgtaccccgaagaggaggtgctcaaccttctgcttaaaaatggtgccgacatcaacgcagcgaatgacgaaggcgtcaccatcctccatatcatcaccgatccagacaccgtttccctgcttgtaaagcgcggtgcgaatatcgaggcccgcgatgagaggggatggacgccattgatcatgcaggcgaataatcagcagaacggccccgatgtggtggctgcgcttctggcccaaggtgcagacccgaatgccgagggcttcgcgggtgagactgctctctcatttgcaaaacaaactggcgacgcggctcttgtccgtatgctgatcgcaagtggtgcaaagaattagcgctggcggtttagagagtgtctgtgccgccgctacaaagaagcgaggctatgcatcacgtcggtcacagcaatgtccgcagcgctgacaggagcattcggtgcgacacatatgtgaggatga

SEQ ID NO: 8 - DNA, 1089 bp, Agrobacterium tumefaciens
  CDS (121)..(966)
cgatccgatgagcacgggtgccgcggtccggacctataatatcatcctttcggaatcgcgcgccgtagccgcggcgctgattgccgtcgaaggctgagggcgtttcgcgcgcggggaaccatggaacccacagaaaatctcgacgtctgcctcgcgacgctgcgcgacaccgatcgtgaccgttatctcgcctgccttttgtcgcctgccgacaagcgcaatgctctggctgccctttacgccttcaacgccgaaatcgcccgtatccgcgattccgtgcgcgaggcgctgccgggtgaggtgcgtatgcaatggtggcgtgaccttgtcgacggcaatccgcacggtgacagccaatcccatccggtggccgcagcgctcctgacgacgatcgaacactatcgcctgccgcgcccggtgctcggcaatatgatcgaggcgcgtatcttcgatctctatgacgatctgttcgaggatcgcaacgcgcttgagggatacgccggcgaaaccgcctcggccctgatccagctcgcaagcctcgtgctttccgccgaggatgccgctcatagcgcggaagcggcgggacatgctggtgtggcccaggccatggccggcatgctgctgctcatgccgcttcaccggcgtcgcggacaggtctatattcccgctgacatgctggccgcggctggtcttgatcgtgaaacctttcttgcaggtcaggacaggcagcgtatcggcatcgccgttgaaattttcgccgctcatgcgctcgatcatatcgagaaggcaagacgcgcgacgatctcgaaaaacctctttgccgcctatctgcccgtcgctttgtccggcccggtcatcgctgccgccaggaaggcgggggcaggggtgttcgacggcgacctgcaattgtcgcagcttcgccggcaatggttgcttgcgaaggcctctttcctcaagcgtttctgatcacagggcgcctgtcaccaggagccacacttcacgcaagcctcgagctttatgaatccagattgggttcgtgtcgtttcgagtcgcgaatcgaagggtttcggtactgagggagcgtcccgg

SEQ ID NO: 9 - DNA, 1146 bp, Agrobacterium tumefaciens
  CDS (121)..(1023)
aaggacatgttcggcaccggcagcggccgaccgagcccgagatgcaggaatgcaagataggcgagtgcgaagggcataccgaacacgaagcgcacaaaggttgcgcctgtcgtgcccatcatgcccttgaggtgtttttgcaacgtcgagcgcaggttctgcaggaacgcgcttgcgaaagtaatgcctatccagagttccatagccttggcggtatcatgcgcgcaaggccgcagccaagcccgattttccgatcaatcgtcgtcttttcgattttcttcgccgttcgaaacaggcataaagcagcccatggacacaaagccgaaaaacctgcccgacgccgccgatttcgcccatgtcagcgaatgggtcttcgatctcgacaacacgctctatccgcatcacgtcaatctgttttcccagatcgatcgtaacatgacagcctatgtggccgagcttctgaagctcgaccccgaagaggcgcgcgtgctgcagaagcgctattatcacgaccatggcaccacgctgcagggcctgatgatcaattacggcatcagcccggacgagtttctggaacgtgcccatgccatcgactattcagcactgaaaccacatccggaactcggcgaagcgatcaaagctctgccgggccgcaagttcattctgaccaatggcagcgtcaaacatgcgcaggccgcagcaggtgcgctcggcattctcgatcatttcgaggatatcttcgatattgtcgccgccaactatctgccgaagccggccagcgcgacctacgagaagttcgcagcacttgcgaagctcgacacaaaaaaagccgccatgttcgaggatttgccgcgcaatcttgcggctcccaaggcgcttgggatgaagaccgtgctgctcgtgccttcgaaccttgagggcgtgatcatggaacgctgggaaatccctgatgtgacggatgagcatatcgactatatcaccgacgacctgaccgggttcctggcgaaaaccatcgcccgctgagcagcgaacattaccgaagcttcatgcaccttacggatgtttaagtggtgtgccttggtcgacgtcactatcttttcttcagggacaccaaagaaaaggtaatgacgatgaccacacgcaaaa

SEQ ID NO: 10 - DNA, 1116 bp, Agrobacterium tumefaciens
  CDS (121)..(993)
gccgcatgggctttgtcgcggcttatggacagggacgaattccgtacatactctgccggtcgcgcaccggagactgatccggaggcggaaatggaatggcaactggcagaggcatgaagcatgcatgtgatgattttcggcgcgggatattcagggaaagccattgccaacgcgttgaaaaccaaggctgcaagtgtctgcggcaccacacgcagcgcggataagttcgcaaacctcgccgccgccggcatgacgccgctgctcttcgacggcgtccatctcgatgacgatctcatcgctgcgatgcgcaatgttacccatctcgtgcaatccatcgctccaggcaaggacggcgaccctctgtttgcgcttctgggcggcgatctgaaaaagctcctgcccaatctgaaatggttagcctatctttccaccgtcggcgtctatggcgaccatcacggcgcctgggtgaacgagacgacaccctgtcgcccggtctccgcacgctccgtggagcgggtagccgccgaggccgcgtggactgaggcagcccaaagcgcagatgtgccgctttccatcctgcgcctgtccggaatttatggcccgggacgcaataccttcatgaatttcgagaagggaacggcgcgacggctggtgaagaaggatcaggtattcaatcgcatccgtgtcgaggatatcggcgcggcaatcgcctttctggcactgaagaacgaacgcggcatcttcaacgtgaccgacgacgaaccctgcccaccgcaggatgtggtgagcttcgcggccgacctaatgggtgttgtcccccctgctgaacaggatttcggaaccgccgatctgacgccgatggcgcgctctttttatggcgaaaacaagcgcgtttccaatgctaaaataagggaactcggctttgatttccgctttccggagtatcggcagtcgctgaagcaattatgggaaagcagcctctggcgtggctgaaaacaaccccgtcaaaaacgacgaaaaatccatccaacgccgttttggcggatagttttcgttgtatttaagcgaattaagacttaattaactacggcgaatcgctaaaatcctactcattgg

SEQ ID NO: 11 - DNA, 1191 bp, Agrobacterium tumefaciens
  CDS (121)..(1068)
acttttttctcccgcaaggatgtcgaggtgagactggcggcttgagatggtgtccagcagagggtatcaggtgctccgatcctcgccggaccttcggcaagcaacaaggtatatcctgacatgacccccgacttcctcgtcacccattccggcggctttcacgccgacgaactgctgtccagcgtcgtgttgacacggctttttccgcaggcccggctcgtccgcagcagagcccccgaatggataacgcctgatgcagaccgcatcatctacgacgtgggcggcgcttatgacgccgaaaaatgcattttcgatcaccatcagcgtggtgcgccgctgcgcgaggacggtcagccctatagttcgttcggcctgatctggaagcatttcggacgcgactaccttgccacatcaggcattcctgaagatcatgtcgaaactctgcacgcgtctttcgatgcaggatttgtattgccggtggatctggtcgataacggcgccctcagcccctccattgccggaccacttgccacgctcacgctgcccgtcctgctggagactttaaagcctgtcttcgatgacaccgatcccgaagcggacgatcgcggttttatagccgcgctggcagtcgcgcgcagcttcgtcgaggcgaaactggccacaggcgctgccaaattgcgcgccgaagcactggtgcagaaggcgatcgccgatactggcgaagggcatatccttgagctgccgatgggcatgccatttcggtccgccatcgtaaaagccggcgccgaccacctgctctttgtcgtccatccccgggacaacgactggtgccttaccggcatccgccgcgcggatgaaggttttgaactgcgagccgacctgccagcggcctgggccggcctgacgggcaaagagttggaagcggtttgtggcgtcgaaggcgcgagcttctgccacaatggccgctttatcgccgcagccaaaacccgcgaggcgattttgaccatggcggaactggcagtgaaagaggctctcgcctccgcgcaaacaacagcggcgaagcaataacattgtgcacaggctaaacggttgccgtccggttcttttcaggcggatggcgaccgtttcacacccttatggcaggcgcgctcagggccgcggtcaggacatgtccaaagcaagggtcagatc

SEQ ID NO: 12 - DNA, 2043 bp, Agrobacterium tumefaciens
  CDS (121)..(1920)
cgagggcgcaaggcgttccattgccgccggcgacgtgtttttcggatgaggtgaacggtgcggcccaggaacccgcgccgaaccggatcccacaggttggagcttcctgcagctcatgaaatgcagggtgacaaaccggttccgcatcggcccggtcgatcagatggttcaacaggtccgatgctggataaaggtggcgggcgcggcaaattgtcgtcgcctgagcgctttcgtgaatggacaagaaaaacaatggctaaacaggacgaactggtctttgtgccgcttggcggcgttggtgaaatcggcatgaatctggcgctttacggttatgggccggagagcaagcgccaatggatcatggtggattgcggcgtgactttcgccggcccggacctgccgggcgtcgatcttgtgctgcctgacatcagctacatcgccgagcagaaaaagaacctcaagggcatcatcatcacccacgcccatgaggaccattatggtgcaatgaacgatctctggccgggtctcaatgtgccggtctatgcctcgcctttcaccgccggcatgctggaagccaagcgcgcctatgagggtacccgtgccgaaatcccggtgacgattttcaaggccggcgaccgcatcaatgtcgggccgttcgaaatcgaggctgttggcgtcaaccactccattccggagccgatgtcgctgatcattcgcacgccgcttggcaatgtcatccacaccggcgactggaagatcgacgaggccccgtcgcttgggccgttgacggatgaagcccgtttccgcgcgatcggtgaagagggcgtgctggcgctgatgtgcgacagcaccaacgcgctgcgcgacggcgtgtcgccctccgagcatgaggtttccgaaggcctgcgccagatcatcgaaagtgccgaaggccgcgtggccgtcaccactttctcctccaatgtcgggcgcatccgctcgattgcgcaagcggcggaggcggccggccgcgaggtattgctgctcggctcttcgctgaagcgtgtcgtgaatgtctcgcaggatcttggcatcatggaaggcatcaagccgttcctcgccgaagacgaatatggttatattccgcgcgaccgtgtggtggtgatcctgaccggttcgcagggcgaaccgcgcgcagcgctcgccaagctttcccgcgacgagatgcgcaatgtggcgctcgcttcgggagataccgtcgtcttttcctcacgcgccattcccggcaacgaaaaggcgatcatcgagatcaagaacggcctgatcgaacagggcgttcacatcgtgaccgacaatgaagctctggtgcatgtctcgggccatccgcgccgcaacgagcttttgcggatgtatgagtggacgaagccgcagatcctggtgcctgtccacggcgaggcggcgcatctggtggcgcagaaggaactggccgagcaggccggcatctcgcaggtgccgaaggtgcgcaacggcaatgttctgcgcctggcacccggcccggcggaagtcatcaacgatgtgccgcatggacgcgtcttcaaggacggtaatctcgtcggcgatatggacgagatgggcatcagcaatcgccgcaaactgtccttcgccggccatgtcgctgtcaacgtcgtgctggacagccgttacgacttcctcggcgatcccgatgtcgtgccgttcggcctgccggaatttgatgacgaggacgaggcgatggaggacacgctttatgacgccgtactcggcgccgtcgagagcattccgcgcgcccgccgcaaggacctggcactgctgcgcgaatcggtgcgccgcgccgtgcgcgccgctgccaaccagagctggggcaagaagccgatcgtcacggttttcgtaacgaaggtctgatccgggaggtgagggtggggcttacgaatcctccgtcgccgcttcgttgaattagggggccgtgccgaaaagcgcggccctctttgtttctttgtggctttttgcgcttcttgccgagaattg

SEQ ID NO: 13 - DNA, 2616 bp, Xanthomonas campestris
  CDS (121)..(2496)
gccggcgcttgcgttccatcgtcgcaacgacgccgcaacaccgccgagcgcaaggccgaccaccgcagactgcaaccgcgtgcgcactgcgccgttgcctacaggagctaagctggtccgatgacgcatgcacaccccaatgatgacgtgaacgatgctgcgctgagcctgtcgcggcgtcggtttctggcctggagcagtctggcggtggcggccggattcctgcgttttccagcccaggccgccgcggcgcagcccggcagcatccgcgcggtgccgctggcgcaggtgcggctgacgccgtcgctgtttctggatgcgctgcataccaatcgccgttatctgatgcgcttgcagccggaccgcttgctgcacaacttcgtgctgtacgccggtctcgatcccaaggcccccgcctacggtggctgggaggcggacaccatcgccggccatacgctgggccactacctcagcgccttggcgctgatgcatgcgcaaaccgacgatgcacaatgccgtacgcgcgccagctacctggtggccgaactcgcacgctgccaggcgcatgccggcgatggctacgtggccggcttcacccgcaagaatgcggccggccagatcgagagtggccgggcggtattcgacgaactcaaacgcggcaagatcgagccgctaccgttctatctcaacggcagctgggcaccgctgtacacctggcacaagctgttcgccggcctgctcgacgtgcatgtgcgttgcgacaacgcgcaggcactgcaggtggcggtggcactggccggctacctgcagggtatctttgccgcactggacgacacgcagctgcagaaggtgctctcgtgcgagttcggcgggctcaacgagtccttcgtcgagttgcacgtgcgcaccggcgatgcacagtggttggcactggcgcagcgtctgcatcatcacgctgtgcttgacccgttgatcgcccagcgcgacgaactggtgcaccagcattccaacaccaacatccccaaactcatcggtctggcccgcgagtatgaagtgaccggtgatgcggcttccggcgcggcggcgcggttcttctggaacaccgtcaccgaccatcacagctatgtaatcggtggcaatggcgaccgcgaatatttccagcagccggacagcatctcgaaatttctcaccgagcagacctgcgaacattgttccagctacaacatgctcaagctcacccggcacctgtatcaatggggcccgcaagccgcgtacttcgattactacgagcgcacgctgctcaaccacgtgatggcgcagcagcatccacgcaccggcatgttcacctacatgacaccgatgctggccggcgaggcgcgcggctggtcatcgccgttcgatgatttctggtgttgcgttggcagcggcatggaggcgcacgcccagttcggtgactcgatttactgggaagacgggcagggcgtggcgatcaatctgtatgtgccgtcgcgcgtgcgcaacgcagccgggctggacatgaccttgcacagcgcgctgccggggcaaggcagcgtctcgctgcgcatcgacgctgcccccgcagcgcagcgcacgctctcgctgcgggtacccggctgggcggccgcgccggtgctgcagctcaatggcgcggtcgtggacgcagcggctgtcgatggctatctgcgggtcacgcgcatctggcacccaggcgacacgctcaacctcagcctgcaaatgccattgcggctggaagccacgcccgatgaccccgcctgggtctcggtgctgcgcggaccactggtgctggcggcagatctgggcgatgccgccacgccgtggtcgggcaagacgccggcgctgatcggcggtgacgaggtcctgcagcagctgcaaccagcggcggggcagggcagctacgtctacagcgatggcgcccaacagtggcgcttctcaccgttctacgcgcagttcgaccgccgcagcgcggtctacctggaacaccgcagtgccaacgactggcagcaacgccaggccgaacaggacgccgcagccgcacgccagcgtgcgctcgatgccagtgcactggactgggtcgccctgggcgacgacgcgtctgaaaaagcgcacgccctgcagagcgacggctcctacgcactgagctaccgccgccgcgccggccgcgacgcccgcaccggtggcttcatcgcctttaccctgcgcaacaccgcaaccgcccgcgtgctgcgcctgcgttactggggcgacgaaacccgccgccgcttccgcatcctcgccgacggcgaactcattgccaacgaacgcctggacggcaaccgcggcctggacttcgtcgatgtggattacgccctgccagcagcggtggcaggccgcgccatcctgcagatacgcatcgaaccagaaaccggctactcggccggcccggcgttcgggtgttgggtgctggagtcggcgtgagtgccgaagtgtgcgcgctgtcgatcttgctgcagcgtgtcttgtagagggggaaggtctactacagacggtaaagtgaaagcgcacagtaatcccgcgcgaactttccggcgcgcaaaa

SEQ ID NO: 14 - DNA, 1122 bp, Xanthomonas campestris
  CDS (121)..(1002)
tggttgcgttccgctcactgcagtggcgagcacgttcaagatccccgtcacttttatagagggcgtgtgttgcatttgtcttacgcgacacacgctgacacacagctgccacatgtcggcatgcactgtagcgggatgacaaacacctccattcccgccgatttgggcgactggatcattgcccaggccatcgccggacacccgcccgagcacacgctgcaaccgctgctggaagcgggctggagcgagcaggacgccatcgatgcggtggaagcgctggtacgcgcgcatgtgcaggcacacgcgcaggcgcacggcctgccggtgccggtgccggtgcgcgtgcccgcaccgctgcaggccgacgcgtcgtcgctgctggatctgggcgatcgccaggtgcaggtactggtcagcctgatgctgccgcgcgtggtggtgctgggcggcctgctcgccgatgacgaatgcgatgcattgatcgcactggcacgcccgcaactggcccgctcgcgcaccgtggacaaccgcgacggcagcgagatcgtgcatgctgcgcgcaccagccacagcatggccctgcaaccgggccaggatgcgttatgtcaacgcatcgaagcgcgcattgcgcagctgctggaatggccggtggagcacggcgaagggctgcaggtactgcgctatgccaccggtgcgcagtacgcgccgcattacgattacttcgaaccggatgcccccggcacgccagtgctgttgcagcacggcgggcaacgcgtcgcctcgctggtgatgtacctcaacacgcccgaacgcggcggcgccacgcgcgttcccgatgtgcatctggacgtagccgcggtgaaaggcaatgcggtgttcttcagctacgaccgcccacatccgatgacacgcaccctgcacgccggcgcaccggtgctggccggcgagaaatgggtggccaccaaatggctgcgcgaacgcccgctgcacgcaccgacctgatcggtgctgcagcgtgcccgcatgcgtgggctttcgtggcggtgttagcggctgccgaacacgcgctggtcgatgcgcagcatcaccaggcgcagcggcgtccacgccaggcacaggccg

SEQ ID NO: 15 - DNA, 1131 bp, Xanthomonas campestris
  CDS (121)..(1011)
agctcgtccccatcacctcatcacgtgccgcacgcacagaacattccatcttgcgttgcagcgcgtccacgccgcgggctgcatggcaagctgccgctctgcccactgcacgagccgcccatgaccacgttgatcgctccccgtgtccacgatatcggcggcttgcaggtgcgccgcgccgtgccgaccctgcaggcgcgcagcgtcggcccgttcgtgttcgtcgaccacatgggcccggcagtgctggagcccgatcacggcatcgacgtccgcccgcatccgcatatcggcctggccaccgtcaccttcctgtggtcgggcgagatcggccaccgcgacacgctcggctcggatcaggtgatccgccctggtgacgtcaactggatgaccgccggccgcggcatcgcccattccgaacgcacgccgggccccgaacgcgcacgcgagcacgccatgcacggcatgcagacctggatcgcgttgccgcgctcggccgaagagaccgcgcccgccttccatcaccacgccgccaccagcctgccgcaacagcgccgcaacggcgtgtggctgcgggtgatcgctggccgcgcctatggcgaggaatcgccggtgcaggtgttcagcggcacgctcaacgtggcgctggatctggacgccgatgccgagatcgatctggacaccggccacgccgagcgcgcgctctacatcctggagggcgaggcgcaactggatggcgccgacgtgccggcgcggcatctgatcgtgccctctgccggcacgcgcggccggctgcgtgccaagacccccctcaaggcgatgctgctcggcggcgagccgctggatggcccgcgccacctgtggtggaacttcgtctccagctcacaagagcgcatcgaacaggccaaggacgattggcaggccggccgcttcggcaccatccccggcgacgatcaggaattcattccgttaccggaaacgccggcgcccaaacccgtcaattacccctgaaaccatgcgcggattcgcactggcgaacacaaccgtgccaggtctcgcaacgggggtgccacgccacacaagcgatgccagacgcaactgcgacccatcttgagcacgcccacaaaactg

SEQ ID NO: 16 - DNA, 1377 bp, Xanthomonas campestris
  CDS (121)..(1257)
gctttttacgattcccgattccccactcccgattcccacccctctgtcacccccgcgacttcttcgcgcaataccgctgccatcgcgttcccggaagctagatcaaacgggagaacgaccatgcgctacgcgatcgttaccgaaacctatccacccgaagtcaacggcgttgcgctcaccgtgcatggcctggaaaccggtctgcgtgcgcgcggccatcaggtcgatgtggtgcgcccacggcaagcggccgacagcacgccggccgacaccgtgctggtgcgcggcgcgtcgctgccacgctaccccgggctgaagttcgggctgccggccacccggcggctcacccggcattgggcaaccacgcaaccggatgcgatctacgtggccacggaaggcccgttgggatggtcggccatgcgcgcggcgcggcggcttggcattccggtcgccaccggcttccacacccgcttcgacgaatacctgcccgattacggcgcggcctggctgcaaggcaccgcgctgcgctggatgcggcgcttccacaaccaggccgaagccacgctggttcccacccgcgagctgcagcagttcctgcgcggcagcggtttcgagcgggtgcagttgctggcgcgtgcggtggacagcctgcagttcgatccgtcccgccgcgacgccgcgttgcgcgccgaatggggcatcgaaggcgaaggctttgcagcgatctacgtaggccggattgccaacgagaaaaacctgcctctggcgatccgcgcgtttcgcaagctgcagcagctccgccccaaggcgcgctttgtgtgggtgggcgatggcccggcgcgcgaaaagatcgctcacgagaatccggacttcattttttgcggcatccagcgcggcgacgcgctggcgcggcacttcgccagcggcgatctgttcctgtttcccagccgcagcgagaccttcggtaacgtcaccctggaagcgatggccagcggcgtggcgacggtggccttcgattacggtgcggcgcgcgaatacctgcgcaatggccagaccggcgctgcagtggaggacgacgctgcattcgtgcaggccgcactgacactgaccgaaaacgacgacgtgcgccaacgcatgggccatgccgccgcacaggcgatgaagaaactgcacccggacaacgtggtgtccgatttcgaagcactgctgctggggatcacggcggcacgggggcggtatgtcgtcgacgcggcttgaactcctgcacggtcacgaagcgcgctggtgccggcgggccaaccactggtgccgccggcacccggtgcggcgcagcttcgcggcgatcagccggctcggcgacggggtgttctggtacag

SEQ ID NO: 17 - DNA, 1716 bp, Xanthomonas campestris
  CDS (121)..(1596)
gcagctggcgttgcaccactgcctgcggtcgccccggacgatgccttgcacctgtggtatcgcgagcctgctaatgaatgggtgcaggccttgccggtcggcaacggtcggctgggcgcgatggtgtggggcggcatcgcccacgagcgtctgcaactcaacgaagacacgctgtatgccggtggtccgtacgacgccaccaatccgcaggcgctggcggcgttgccgcaggtgcgcgcgctgatctttgccggccgctatgcagaggccgagcagctggccgatgccaagctgttgtcgcgtccgctcaagcagatgccgtaccagccgttgggcgatctgttgctggatttcgaccgtgccgacggcatcagtgagtaccgtcgtcagctcgatctggataccgcagtggccaccaccacgttccgctccggcggcgcggtgcaccggcgcgaggtgttcgtgtcggcgcagtcgcagtgcatcgtggtgcgtctgtcctgcgatcggccgggtggcatttcgttgcgggtgggcatcgacagcccgcaaagcggcgaggtcacggtggagcagggcggcctgctgttcagtggtcacaatggcagcttcgccggcatcgacggcaagctgcgctttgcattgcgcgtgatgccccaggtcaagggtggcagcgtcaccgcggtgcgcgaccgtctgcgtatccagggtgccgatgaagtggtgctgctgctggccgcagccaccagctaccaacgtttcgatgcggtggagggcgatccgctcgcgctcaccgcggcaagcctgcaaaaagccggcaagctcagctacgccgcgttgttgcgcgcgcatctggccgatcatcagcgcctgttccggcgcgtggtgatcgacctgggcagcagcgaagcggccacgcttcccaccgacgagcgcgtgcagcgctttgccgagggcaacgacccggcactggcagcgctctatcaccagtacgggcgctatctgctgatctgcagttcgcggcccggcactcaaccggccaatttgcagggcatctggaatgacctgatgcagcccgccgtgggagagcaagtacaccatcaacatcaacaccgagatgaactactggcccagcgaagccaacgcactgcacgagtgcgtggaaccgctggaagccatgctgttcgatcacgcccgcacaggtgcacacaccgcccgtgcgatgtacgacgcgcccggctgggtgggccacaacaacaccgatctctggcgccaggccgggccgatcgacggtgcgcagtggagcctgtggccgatgggcggggtgtggttgctgcagcagttgtgggaccgctgggactacggccgcgaccgtgcgtatctggccaagatctatccgttgttcaagggggcggcagagttcttcgtcgccaccctggtgcgcgaccccggcaccggcgcgatggtgaccaatccatcgatgtcgccggaaaaccagcatccgttcggggcggcggtctgcgccggcccgaccatggatgcgcagctattgcgcgacctgttcgcgcagtgcatcgcgatgagcaagttgcttgatgtcgatgccgcgctggcacagcagctggccacgctgcgcgagcaattgccgcccaaccgcatcggcaaggcggggcagttgcaggagtggcagcaggactgggacatgcaggcgccgga

SEQ ID NO: 18 - DNA, 1179 bp, Xanthomonas campestris
  CDS (121)..(1059)
catttttgatgaccagtgggaaaaggaccgacaaggctttggcgcgaaacgtttccatgggctgaatcgcctgctgccctgaccggcaggttttatttcattctttatcaagacatccgtatgagcgcgcgcatccccggcctggacatcacccgtttcgacagcgaccgcgaggcctttgtcgccgagctcggtgccgcttatcggcaatggggctttgccggtatccgcaatcacggcattgcgcagtccgacatcgacgccgcctacgaggtgttcaaggcatttttcgcgctgcccgacgaggtcaagcgcagctaccacgtgcccggcagtggcggcgcgcgcggctataccgcgttcggtgtggaaaccgccaaggactccaagcatttcgatttgaaagagttctggcacatcggccgcgagatcccggacgattcgccgtaccgcgcggtgatggcgcccaatctgtggccgaccgaagtgccgggcttccgcgaacgtggctaccacctgtatcagcagttggatcagctgggctcgcgcgtgttgtcggcgctggcgctgcacatcggcctgccgcaggactacttcgccgacaagaccgatcacggcaattcgatcctgcgcccgatccattacccgccgatcaccagcgacgacatcccgaatgtacgcgccggtgcgcacggcgacatcaatttcatcaccttgctggtgggcgccagcgccgccggcctggaagtgcgttccaacgaaggcgagtgggtgccgttcactgcggacgccgacaccatcgtggtcaatatcggcgacatgctgcagcgcctgactaaccatgtgtacccgtccaccatccaccgcgtggtcaatccgccgggagaggaagcgcgcaagccgcgctattcggtgccgttcttcctgcacccgaacccggatttcctgatcgacgtgttgccgtcctgcatcagcgccgacaaccccagccgctatcccgagccgatcaccgcacacgggtttctggaagagcgcctgcgcgagatcaagctcaagtaagccgaacggacccaagcgcagcctatggccgcgctcgcaacatggcggatgacgctcgccacagtttgcttgatgaacgttcgctcgcgtccggttaaagcttgcgttcacctctgccga

SEQ ID NO: 19 - DNA, 1275 bp, Xanthomonas campestris
  CDS (121)..(1155)
tgcagccaccgggcactgccgcccaagtggcatgaacggactgcaagccttcgcacctgggtgcggagatggcccaccgccggggcgttgccccaggcatcagatgagtaagtgaccggcatgaccgacagcgttcgttttcctgcggaatgggaatcccaatccgccattctgattgcctggccgcatgccggcaccgactgggccgagcgcctggccgaggtggaagacacctatattgccctggtcaccgcgatcacccgcttccagccggcgatcatctgcgtggccgacgatgacctgcagatctatgccgaagcccggctgcgttcggcgcgggtggagatggcgcaggtacgcttcgtggttgccgcctacaacgacacctggctgcgcgattcgggcccgatcacgctacgccatggcgacggcttccggctgatggatttccgattcactgggtggggtggcaagttcgaggcgagcctggacgatcaactggttagcagcctcgatgcagcgagcgtcttcgtgcccgccgaggtgcagacggtcgactttgcgctggaaggtggcgcgatcgaaaccgatggcgccggcaccttgctgaccacctggaagtgcctgcacgaacggcacccgcagcgctcgcgcgactccctgagcagcgacctggcgacctggctggcgcaggaccgcgtgctgtggctggaccacggctatctggaaggcgatgacaccgacgcgcatatcgatacgctggcgcgctttgccggcccggatgcgatcgtctaccagggctgcgacgtaccgagcgattcccactacgccgaactgcaggcaatgggcgaagaactggctgcgctgcgcaaggctgatgggcagccgtatcgcctgttcgtgctgccgtgggcagagccgatcctcgaccaaggccggcgccttgcggcgtcgtacgccaattttttgatcgtcaacggtgcggtgctgatgccggcctatggcgacgccgccgatgctgcggcgcaggcggtgctggccgacgcgtttccgcagcaccagatcgtgccggtgccatgccgcccgctgatctggcagaacggcagcctgcactgtctgaccatgcagttgcctgccggcctgctggccgcctgagccgtattgttccggccggcaggccggcgttccaccatggcgcgctaccctgtgcgccacctagcaggacgagtagccatgacccgacatctccttcccgtcgcgctgatccaggagcgc

SEQ ID NO: 20 - DNA, 2187 bp, Xanthomonas campestris
  CDS (121)..(2067)
ctggttctggatgcatggcggcatggaatacccgctgatgtgggcgctggtcttgctggcgctggtatgcgccggcggtggcgcgtggtcgctggacggcctgcgtcggcggcacgtgtcatgagcacgcccactgattcccacgatgccggccggcgccagctgttgcagggtgccgccgccggccttgcactgggcattgcgcctgctgcgttgactgccaacaggacgcctccgatggtcgatctcattctccgtaatgcacgtatcaccacgcttgacccgcgccaacccagcgccactacgctggcggtggccgatggccgcatcgtggccgtaggtgacgacgcgcagatcatggccatggccaagcaggcgcgtgtcatcgacgcgcagcagcggcgcgtggtgcccgggctcaacgacagccacacgcatctcattcgcggcgggttgaactacaacctggagttgcgctgggacggcgtgcgctcgctggccgatgccatggcattgctcaaggcgcaggtcgaccggacgccggcgccgcagtgggtgcgcgtggtaggcggcttcaccgcgcatcagttcgttgaaaagcgcctgccaaccctggacgagatcaacgcgatcgcgccggacacaccggtgttcctgctgcatctgtacgaccgcgccttgctcaatcgcgcggcgttgcgtgcgtgtggctacgacaagaccacaccggatccgcccggtgggcgtatcgagcgcgacaagctcggcaatcccaccggcctgctgctggccaagccgaacgcgctgatcctgtatgccagcctggcgaaagggccgcggctgccgatcgaataccaggccaactccacgcgccacttcatgcgcgagctcaaccgcctgggcatcacctcggtcatcgacgccggtggcgggttccagaactatccggaggactatcaggtcatcgagcagttgcatgcggccgggcagttgagcgtgcgcatcgcctacaacctgtttacccagaacaagggcaaggaagtcgccgatttccgcggctggaccgaggtgttgccggtccgcaagggcgacgatctgctgcgccacaacggtgccggcgaaatgctggtgttctcggcggccgatttcgagcagttcagtgagccgcgcccggagcttgcgccggagatggaaccggaactggagcaggtggtgcgcctgctggtggaaaaacgctggccgttccgcatccacgctacctacgacgaaagcatctcgcgcattctcgatgtctacgaacgcgtcaacgcggacatgccgttcgatggcctgcactggttcatcgatcacgccgaaaccatcgcgccacacaacatcgatcgcatccgcgcgttgaacggcggcatcgcggtgcagcaccgcatggcctatcagggcgaagccttcgtacagcgctacggcgtgcaggccgcgcgccatacgccgccggtgaggcgcatgttggcggccggtgtgccggtgggtgcaggcaccgatgccacgcgtgtggccagctacaacccgtgggtggcgctgtcgtggctggtcaccggccgcaccgtcggcgggctggcactgtatggcgacgagaatctgctcgaacgcgaacaggccttgcgcttgtggacgcagggcagtgcctggttctccgatgatgaagacaggaagggcaccttggcacccggtcagttggccgatttcatcgtgttgtcggatgactatttccgcgtcgacgatgccgcgatcgccgatatcaccagcgtgctgaccgtgttgggcggtgccgtggtgcacggtgacggcgacttcgctgcacttgcgcccagcctgccgccggcaatgccggaatggtcgccggtcaaccgttatggtggctatcacgtgggcgggttggcaagcgtggcgcatgcgcatggccctggctgccgcacccacggcgcacatgggcatgccgcgcaccacgccgcacccagcgacgatctgcgcggcttctggggggcattgggatgcgcatgtttcgcgttttaaggccgccgcagcgcctggatgctgcgaggggtgtgtgcgtcgttgcagcgcaggtctgcaaccttgagaaggcgtgtgcagtgtccgtcgcgggcgcctggccggaccgcgcgttttttc

SEQ ID NO: 21 - DNA, 3747 bp, Xanthomonas campestris
  CDS (121)..(3627)
aactagccatcaatggttcgtttagtgtacgccgccggcgccttgcaagccaggggcaaagccctcgcaaaccgttcccaacggcgcctgatgctccgttgaccgagatctctagcgtccatgggtctttccacgatcaagctgtccggttccaagtcgtttggtcgaacccgaccacgttgcacctgccccaccaacatgaccggcatcgtcggccccaatggctgcggcaagtccaacatcatcgatccggtgcgctgggtgatggcccaaagctcggccagccgtctgcgcggcgactcgctgaccgacgtgatcttctccggctcctcggcgcgcaagccggtgtcgcaggcgacggtggagctgatcttcgataactccgatcacaccatctccggcgagttcgcctcgttcaacgagatctcggtcaagcgcctggtcagccgcgacggcaccagcgcctattacctcaacggcaccaagtgccggcgccgcgacatcaccgacctgttcctgggcactggtctggggccacgcagctactcgatcatcgagcagggcatgatcagccagatcatcgaggcgcgcccggaagatctgcgcgtctacttggaagaagccgccggcatttccaagtacaaggaacgccgcaaggaaaccgaaacgcgcatccgccatacgcgcgaaaacctcgatcgcctgggtgacctgcgcgaggaaatcaccaagcagctggcgcatctgcagcgccaggcgcggcaggccgagcagtaccaggcgttacaggaagaacgccgcatcaaggatgcggagtggaaggcgctggaataccgcggcctggatggccggctgcaaggcctgcgtgaaaagttgaatcaggaagaaacccgcctgcagcagttgatcgccgagcagcgcgatgccgaagcgcgcatcgaaaccggccgtgtgcgccgcgaagaggccgccgaagcggtggccaaggcgcaggccgatgtctaccaggtcggtggcgcactggcgcgtatcgagcagcagatccagcaccagcgcgaactctcgcatcgcctgcacaaggcgcgcgacgaagcgcagagccagctgcaggagctcacccagcacatcagcggcgactccgccaagctggcggtgttgcgcgaggcggtggccgatgccgaaccgcagctggagcaattgcgcgaagatcacgaattccgccaggacgcactgcgcgaggccgaagcccgcctggccgactggcagcagcgctgggaaacccataaccgcgacaccggcgaagcctcgcgcgccggcgaagtggagcgcacccgcgtcgattatctggaccggcaatcgctggaagccgaccgtcgccgcgaggcgctggtcaacgaacgcgccgggctggatctggatgcgttagccgaagcgttcgaacagatcgaactgcggcatgaaacgcaaaaggcctcgctggacgggctgaccgagcaggtcgaagcgcgcaagcacgcgctgggtggcttgcaggaacagcaacgcgccagccagggcgaactggccgaagtccgcaagcaggcgcaggccgcacgcggccggctgtcgtcattggaaacgctgcagcaggcagcgctgggtcaggagcagggcgccgccgtggcctggctcaaggcgcgcgggctggattcggccgtgcgtgtgggcgagcgcatcagcgtggaaagcggctgggaaaacgcggtggaaggcgcgttgggccagctgatcgaaggcgtgctggtggaggcgccggaacaactggtcgacgcgctgggtgagttgggcgacgggcgcatcgcgctggtgtcgtcggctaccgacaacgtgcagttcgcgccgacctcgcttgccgccaaggtgcaggggccgattgcgatccgccgcttgctggcgcgcctgcatgccgccgaagacctggatgccgcgcgcagcctgcagtgcaacctgccggacggcgattcggtgatcacccgcagcggcgagcggctgggcgagggctgggtgcgcgtgtcgcgctccggcgcggccaagcagggcgcgttgctgcgcgagcgcgagatccaggagctgcgctcgcagatcgacacgctgcaggagcgggagggcgacctggaagaacaactggccagcttccgcgagcaactgctggccgccgaacaacagcgcgaagacgcgcagcgccagctgtacatggcgcaccgcagcgtgtccgaactggccggccagctgcagagccagcagggcaaggtggacgcggcacgtgggcgcatcgagcggatcgaggccgaattagggcagttgctggaaaccctggacaccagccgcgagcaggcccgcgaagcgcgcgccaaactcgaggacgcggtggtgcgcatgggcgacctgcaaggcacccgcgaagcgctggaaagcgaacgccgccagcttaccgatgcacgcgaccaggcccgcgatgccgcgcgcggcgtacgcgatgcgatgcacgccttggcactcacgctggaatcccagcgcacccagatcacctcgctgagccagaccctggagcgcatggacagccagcgtggccagctcgacacgcgcctggaagatctggtggcgcagctgagcgaaggcgattctccggtcgaaacgcttgaacacgaacatcaggcagccttgagcgagcgcgtgcgcaccgagcgcgtgctcggcgaggcgcgcaccatgctggacagcatcgacggcgaactgcgcagcttcgaacagacccgccagcagcgcgacgagcaggctctggcgcagcgcgagcgcatctcccagcgcaagctcgaccagcaggcgctggtgctcaacgccgaacaactggccgccgcggtggtcaaggccggcttcgtgctcgaagacgtggtcaatgggttggaggaggccgccaacccggccgagtgggacgcggcggtggggcagatcgatgcccgcatgcgccggctggagccggtcaacctggccgcgatccaggaatacggcgaagccgctcagcgttccgagtatctggacgcgcagaacgtggacctgacgaccgcactggaaacgctcgaagaagcgatccgcaagatcgaccgcgaaacccgtggccgcttcaaggacactttcgaccgcgtcaactcgggtgtgcaggccttgtatccgcgcttgttcggcggtgggcacgcgtatctggagctcaccggcgaagatctgctcgacaccggcgtgaccatcatggcgcgcccgccgggcaagcgcgtgtcgagcatttcgctgctgtccggtggcgaaaaggcgatgaccgcggtggcgctggtatttgcgatcttccagctcaacccggcgccgttctgcctgctcgatgaggtggacgcaccgctggacgaggccaacgtcggccgcctggccaacatggtcaaggagatgagcgagaaggtgcagttcctgttcgtcagccacaacaaggcgaccatggaagcagcgcgtcagctctccggcgtgaccatgcgcgaacccggcgtcagtcgcctggtgagcgtggacctggaagaggccgcacgtttggcgggcgcggcatgacgtgccatgcttgctggaatgattacacctgtcactcttgcgcggaggcacgctgaatgtccgacatggccatgatccggatcgggatcctgatcgctggactgctgctggtcgcggcca

SEQ ID NO: 22 - DNA, 708 bp, Xanthomonas campestris
  CDS (121)..(588)
cagtccaaccgcgcggttcagccgccggaaaggccagcccgttgcggtggccggtgcgcttgtgcttggcgcgtgggcgcgctgcggtcacaatgagcggccccatcaccggaccctttcatgtcgcgcaaagttgccgcccgcaagtcacgtatccatggcaatggcgtgttcgccgtcgctgcgctcagcaagggcgaacgcatcatccagtacaaggggcgcctgcggacgcatgccgaagtggatgccgacgacaccggcgatgtggaaagcgggcacacctttctgtttacgctgagcgatgactacgtgctcgacgccaactacgaaggcaacatcgcgcgctggatcaaccacagctgcaatcccaattgcgaagcggtgatcgaagaggctgaaggcgacgaccgcagcaaggacaaggtcttcatcgaagccaagcgcgcaatcaagccgggccaggagttgacctataactacggcatcaccctgggcgagcggcacacgccgcgcttgaagaagatctgggaatgccgctgcggctcgaagaattgcaccggcaccatgttgcagcccaagcgctgagcaacggcgcgcggcggtcggcatgacgcggcgcgcacatcggttcacggatgcgcaacacgcgcattcctagggtgcaggctcctttgcagattgaggagttcctcatgacccattcgc

SEQ ID NO: 23 - DNA, 1308 bp, Xanthomonas campestris
  CDS (121)..(1188)
tgacgacatcgcgggtcctcgcagtggtcggcgaccacggccgcctgggttgaccagagcacatggtatttaatagatgataaataggtatctttaaaatctacacaggactggaccacaatgccgctgtcacgcctgctgacctgccttgttgcgagccttgctgcctacgcagttcaagccaaatcgcccatgccgccgccctcaccgatcatgctcagcgagttcattgccgaccaggcaccgacgccacaggcgcacgcctctacgctgttggaaacccgcgacggcacgctgctggcggcctggttcggcggcgcacacgagggtgcggccgatgtcggtatctggctcgcccaacgcggcccgcagcactggcagacaccgcgccgcatcgcagacggcgcacacgcaggcgtggatggcgccgcactgccggcctggaatccggtgctgtttcagtccgccaccgggccggtgcagctgtactacaagctcggcccgaatccgcgcgattggtggggcctgcgcatcacctccagcgacaacggtgcgcactggtcgccaccgcagcgcctgccagacggcattctcggcccgatcaagaacaagccggtgcaattgccgagcggacgcatcctggcacccagcagcggcgaagaccgtggctggcgcgcgcatctggaatggagcgacgacgacggtgcgcactggcagcgaggcctgccgctcaacgacgccgccaccatcggcgccatccagcccagcctgttgctgcacgccgatggccgcctgcaggcgcttggccgcagccagcaaaacaaactgttcagtgccttctccgacgatggcgggctgcactggcagccaatgcaattgctggacgtggagaaccccaacagcggcacggatgccgtgatgctgcacgacggccgtgccctgctcgtctacaacccgggcattgccggcaagcaatggtgggatggccgcggcacgctggccgttgcggtgtcagacgacggcgtgcagtggcgccgcgtgctcaccctggaagacagcccgaaagatgaattctcgtacccggccgtgatccaaacccgcgatggcctggtgcacatcagctacacctggaagcgccagcgcatcaagcatgtggtgatcgacccggcgaaattaaaagtgcgttgattgggattggtgcgctgagtgagcgggtctgatgctccataaagacggttgcagggttcgcgacagctgatacagcgagtggtcaacggaggatgctgcgtggaccctcaaggacatagg

SEQ ID NO: 24 - DNA, 2058 bp, Xanthomonas campestris
  CDS (121)..(1938)
cgttttgcgttatgcggcaatgcagcgtgagcgtggacggcgagggtgaaacgatgtgccattcgccggcgtacgtatgcaggccggtccggttccactctaccttgagcaggcattcccatggcttcaacccttcgcccgatccgttccctgatggccgtggccattgcgctggcggccagcccggccatggccgactcggccttcgatcaaaccgtgttcttcggtgacagcctcaccgacagcggttactacaacccgctgctgccggccgcctcgcgcgcggtcaccggcaagttcaccaccaacccgggctgggtgtgggccgaatacgtgggcgaccacttcggcaccaatgcggcgcccaacggcaatggccagaccggcgacaactatgctgccggcggcgcgcgcatccaggccagttcggtcagcgagctcggcgcagcaccgtcggtgaccagccaggtcaatacctatttggcggccaacggtggtcaggccaacccgaatgcgctgtacaccgtgtggggcggcgccaatgacctgctcgctgcagccaccgcgcctgcccaggcgcaggccatcatcggcagcgccgtgaccgcgcaggtgggcgcggttggcgcgctgcaggccgctggcgcacgttatgtgatggtgccgaccattccggatgtgggcatcaccccgcgcttccgcgccggcggtgccgccgccatggcgcagggcacggccgctgccaccgcctacaacaccgcactgttcaacggcctgcagtcggccggcctgcgggtgatcccggtcgataccttccacatcctgcaggaagtggttgccgacccgggcacttacggtttcagcaacgtcaccggcaccgcctgcaacccggcgctcgcgctgccggcctgcaacccgaccagcctggtcgccgcgaacgcgcccaacacctatgtgttcgccgacggcatccaccccaccacgtccacccaccagatcctgggccagtacgccatctcgctgttggaagcgccgcgcctgcagcaggtgctgacgcgctccgcccaggccggcggccgcgcacgtgccgaccaggtggcctggcaccttgatggcaagcccgaggccgatggcctgcgttggtgggggagcgtgcgtggcgacattcagcgctacgacgatgcggacctgtacgacggcatggcgccggccggcctgttcggcgtggattggaccgccggtgacctggtgttcggcggctttgccggcttcggcggcatggatgccgacttcggcaaccgcaacggcagcttcaagcaggatgacaccacgctgggcggtttcgtcggctggtacaccggcccggtctgggtcaacgcccaggtcagctacagctggctgagctacgacgtggaccgcgaagtccagctggggccagccacccgcgtgcacagtggctcgccggacggcagtaacctcactgccgcggtcaatgccggctattcgctgggcgaaggcaacgtcaagtacggcccggtggtcggcctgacctggcagaagctcaagctcgacggctacaccgaaagcaacgcaagctccaccgcgctgggctatgccgaccaggacatcgactcgctggtgggccgcatcggcttccaggtgcgcctggatggcgcgccggtcaagccgtacctgcaggccacctacgaccacgagttcaaggacggcaccgaagccagcgcctggctgcagtcgatgccggaagtgggcatgtacaccgtcccgggccagaacttcgaccgcaactacgcgaccgttgtgctgggcgcccgcaccggcatctggggcctgcagagcaacatcggcctgagcaccaccaccgcccagcgcagcgcccgcgacgccaccgtgttcgtgaacttcagcggcaacttctaaccgtacccgctgcacccgcagcgctaaggctgtctacgacaagggccgggtaaccggcccttgtcatgtgcgccacgcattgcgcccaaggcctgccggcgcataaactctgcccacgcc

SEQ ID NO: 25 - DNA, 849 bp, Xanthomonas campestris
  CDS (121)..(729)
aaatggccgaattgccgccagaagtacgcaatgtcaccgacccgctggatgcagtgggcaacaccaccaaggcggtgaccaaaggctatgcgatcggctcggcggcactgggccgcgctggtgctgtttgccgactacacccacaacctgcacgccggcaatccgaacgaggtattcgcgttcgatctgtctgaccacacggtcatcaccgggctgttgattggcggactgattccctacctgttcggcgcaatggcgatggaagccgtggggcgcgctgccggtgcggtggtcgaagaagtgcgccgccagttccgcgacatccccggcatcatggccggcaccgccaagccgcaatacgaccgcgcggtggacatgctcacgcgctcggcaatccgcgaaatgatcgtgccctcgttgttgccggtggtggtcccggtgatcgtcgggctgttgcttgggccgcgtgcgctgggcggattgttgatcggcaccatcgtgaccgggctcttcgttgcgatttcgatgacgaccgggggaggtgcctgggacaacgccaagaagtacatcgaagatggtcatttcggcggcaagggcagcgaagcacataaggccgcgatcaccggcgacaccgtgggcgacccctacaaggacaccgcagggcctgccatcaacccgctgatcaagatcatcaatatcgtcgcgttgctgttggtgccgctgctttaagcgctgtgcttgaggcgatctgcggacctcgtgggcccggagtgatgcgtgactccgggcccgggcctttgtcggctggcgacggcgcctggcctctaaggctcctgaggctgcaagcat

SEQ ID NO: 26 - DNA, 1938 bp, Xanthomonas campestris
  CDS (121)..(1818)
ttccggtgcgggctcggcggcaaacacaggagcgctcagcagcaggccggtcatcatcaaggcggatttcaacatacataggctcctggatcgggtcgatcacggtggcggcaggctgtcatgccatcactgaacgcggcgccaaagcgtggtgctggcaggcatgcagtacgcggcgcatactcggctgcaatccggccacatgaacatcgcgacgcgcagcaggccttgtcaggccgtcgagtccgtcttcgccgttccactcgtcgcgcaccgcccttcgttcgtcccgaccggacgacatccattcttttgggaggggtcatgctggaacactacggattgatgctcgcgctgggttgtgcggtcctggccattgtctatggcatgttgtcggcgcgctgggtggtcgcgcagccgagcggcaatgcacgcatgcaggaaatcgccgctgccatccaggagggcgcacgcgcctacctcaaccggcaatacctggccatttccatcgccggcgtggtgttgttcgtgctggtcgggctgtttctgagttggtacaccgcgatcgggtttgcgattggtgcggtgctgtcggggttggccggctacatcggcatgaacgtgtcggtacgcgccaacgtgcgcaccgcagaagccgcgcggcacgggatcagcaaggccatggacgttgccttccgcggcggcgccatcaccggcatgctggtggtcggcctgggcttgctgggtgttgccggctacttcgccatcctgcaagccatcggcctgccgcttgagcagaacctgcacgccttggtcggcctggcgttcggttcatcgctgatttcgatctttgcgcgcctgggtggcggcattttcaccaagggtgccgatgtgggcgccgacctggtgggcaaggtcgaggccggcatccccgaagatgacccgcgcaatccggccgtgattgcagacaacgtcggtgacaacgtcggcgactgtgcaggcatggcggcggacctgttcgagacctatgcggtcaccgtcattgccacgatgctgctcggcagcctgaccctggccgaaaccggaccgcatgcggtgttgtatccactggtgctgggcggcgtgtccattgtcgcttcgatcattggcgcagcattcgtcaaggtgaagccgggcggctcgatcatgggcgcgctctataaaggcgtgatcgtgtccggtgtcttggcggcaattgcctactggccgatcacgcaggccttgatgcgcgacaacgcgcatggcgcgatgtcgttgtatgcctgcgcattgatcggactggtcctgaccgggctgatcgtgtggatcaccgagtactacaccggcacgcaatatgggccggtgcagcacgtggccgccgcaagcacgaccggtcatggcaccaacatcattgccggcctgggcatctcgatgaaatccaccgcactgccggtcattgctgtgtgcgcggcgatctggggcgcgttccattttggcggcctgtacgggattgcgatcgcggccacggcgatgctgtcgatggccggcatgatcgtggcactggacgcctatggcccgatcaccgacaacgccggcggcattgccgaaatggccgaattgccgccagaagtacgcaatgtcaccgacccgctggatgcagtgggcaacaccaccaaggcggtgaccaaaggctatgcgatcggctcggcggcactgggccgcgctggtgctgtttgccgactacacccacaacctgcacgccggcaatccgaacgaggtattcgcgttcgatctgtctgaccacacggtcatcaccgggctgttgattggcggactgattccctacctgttcggcgcaatggcgatggaagccgtggggcgcgctgccggtgcggtggtcgaagaagtgcgccgccagtt

SEQ ID NO: 27 - DNA, 957 bp, Xanthomonas campestris
  CDS (121)..(837)
aggcagatatcaggcgtgtgtcccagcgcagcgccaacccgcgccagtcaccgagcgcgcgcgccaaccaaccgcggtgcccaggcaaagcatgcgaatccgacaacgcgatcgaaccggatgccgcctagggcgtcgcattcggcaacctatccacgcgctcaccctcagggtcagcgtacgcagggcggcgatgccgtctgcagacaacgaaaggagttacacatgcggggtgttgtaagccagctgaaacggctgggaatacggggcatcgtgctgggcatggcgtgtgccatcgctgggtgcggacaatgggcgagctcgttctcacccaccggcacggacctcagcagcatggggcggggcgtggacttctatctgaaagataccgaaggcactccacggccattgcgcagctttcgcggcaaggtgctggtgctgttcttcggcttcaccaattgcccggatgtctgcccgaccaccctggcacgtgccgcgcaagtgaaacgcgcgttgggcgcagatgcggcgcaggtgcaattcgccttcgccacgctggatccgcaacgcgatgttcccaacgtgctgcgcacctacgtcaaagccttcgatcctgacttcatcggcctcaccggcagcgaaggggaaattcgggtggcggcagataatctgaaagtggtgtatgccaaggtgccgatgggcaagacctataccatcgaccattccacccgcagttttgtgttcgatcgcgaaggtaaattgcgcgttggccttacccatgagcagacggttgagaccgttgccgccgatattcgccaggtgctggccttgcagtaagtggcgccatcgtgcgacaccgccccatcccatcccatcccgtgccggcacacacgccgccacgcatcgccaaacgacgtagacggagaacaccgatgagcacacgtttattactgctgg

SEQ ID NO: 28 - DNA, 1461 bp, Xanthomonas campestris
  CDS (121)..(1341)
caatggctaacggagcctaaatatagaaatgttccgtaagcaaacagtgataccggtcgcgacaatgcgtgacgatctagtgtgcgtttacacttgcgcggtctcgaactggacgaacgcatgcacgacgtggattgcatcgtggtgggcgccggcgttgtaggcctggccattgcacgacagcttgcgatgcagggaaacgaggtgttgatactcgaagccgcatcggccattggcacgggtaccagtgcgcgcaacagcgaagtcatacacgccgggttgtattacccgcccggatcgttgaaggcgcagctgtgtgttcgcggcaatgcattgctgtatgcatattgccgccaacgcaatgtgccgcatcaacgctgcggcaagttgatcattgcctgcgatcaggcgcagaccgcgcagctggaggcgttgcaacgcaatgcaaccgacagcgccgcaccgggcgtggtgccattgagcagcgcacaggtgcaggcgcgcgcacctgcgctgcgctgcgcggctgcgctggaatccaccaccaccggcatcatcgatagccacgcattgatgctggccttgcagggcgacgccgagcgacatggcgccacgctggcgttacgcgcgccggtgcagtcgatcgaaccgctggttgccggcttccgcgtctgcaccgccggtggcgaggcgatggggctgacgtgtcgctggttgatcaatgccgccggtcacggtgcgccggcgttggccgcaggcaccgaaggcctgccagccacggcgcgcgtgcgcgcacaatatgcgaagggcagttacttcaccttggccgggcggtcgccgtttcatcaattggtgtatccgttgcccgagccaggtggcctgggtgtgcacctgacgctcgatctgcaggggcgcgcacgcttcggccccgatgtggagtgggtggcgcagcccgactatcactgcgagccggcgcgcgccgagcatttctatgcggcaatccgccgttactggccagcgctgcccgatgacgcgctgcaaccgggctactgcggtgtgcgcccgaagatcagcggccccggcgagccggccgccgacttccggatcgatgggccggcgctgcacggcatggcggggctggtgaacctgttcggcatcgagtcgcctggcctgactgcctgcctgagcattgccgagcatgtggcagcgctgctgggtgatgaggcgcgggccaccgccatgcgggtgcttccagtcaaggtggttctgcgcaggggggcgccgcatgttcctgatccaggtgctgctgccgctcgccgacaacaatggcgtgcgttttgagcaggcaatgttcgacgaggtccatcaccacctggtcatgcggtttggtggcatcactgcctacacccgggcaccggtgcatggcgcgtggcaggagcagggcgcgcagctggtgcacga

SEQ ID NO: 29 - DNA, 927 bp, Xanthomonas campestris
  CDS (121)..(807)
aagcaatccacgacgccgccatggcggtgagcaagcgcgccattcacaaggcgaattaagccgggaatcggggagtggggaattgggaatcgcaagagcgttcccatcacttcgctcttcatgctgacgccggacctaggtccggcgtcatgcgttccggcttttgtttctgcttttacccattcccaattccctactcccgattctcatcccatgaaaagcatctgcgtctactgtggttccaatgccggcaacaagcctgcctatgccgagcgcgctatcgccctgggtgaccgcatcgccaaacaggggctgcgcctggtgtatggcggtggcaatgtcggcctgatgggcacggtggccaacgcggtgctggccgcaggtggcgaagtgaccggcgtgattccgcagcagctggccgattgggaagtggcgcatcgcgggctgaccacgctggaaatcgtcggctccatgcatgagcgcaagatgcgcatgttcgagctgtcggatgcgttcgtcgccctgcccggtggctttggcaccatggaggagatcttcgagatgctgacctggcggcagctgggcatcggcaacaagccgtgcgcgttcctggacatcgaaggcttctacgcgccgctgatcggcatgatcgatcgcatggtcgaggaacgcttcctgcatccggatcagcgcagcgacctgtggtacggcgccgacatcgagcagatgctggagtggatgcagcactacacgccggcacaggcatcgaagtggatcgacgagaagcgccgctccaccctggtgtgatcgtcacggcggcaacaacagtcgctgcggaccagcgcagcgcgcttgtattacggcgctaaccccacgcagatgctgtactgatctagcaccacacgctcgtcagtgaccatgcggata

SEQ ID NO: 30 - DNA, 840 bp, Xanthomonas campestris
  CDS (121)..(720)
cgctcccgttggctcgatggctggggtgcattttccgggattgcatgtgatggcggcaaatcgcgttgattcatccgccgctgccggcgcgctccgacacgcgcatgctagcgtttgcgcatgaacatacgcgttgcactgccgcaagctcaggtccactggtgtcgcggctggttgcaggccgcgcacgccgatgcgctgatgcaggcgctgctggaccaggtgcagtgggaggtccatcgcatccgcatgttcgggcgggtggtggattcaccgcgtctgagcagctggatcggcgatgcggatgccagctatcgctactcgggcacgcagtttgcgccgcagccgtggctggaggcgctacagccagtgcgtacgcgtctgcaggatgagaccggctccccgttcaacagcgtgctggtcaaccgctaccgcagcggagccgacgcgatgggctggcatagcgatgacgagccggagctgggcgcgcaaccggtgatcgcatcgctaagcctaggcgcggcgcggcggttcgccttcaagcatcggcacgatgcggcgttgaagcagaccctggaactcggccatggcgacttgttgctgatgggcggcgatacccagcgccattacaaacatgccttgccgcgcaccgtaaaaccggtcggcgagcgcatcaatctgacctttcgccagattgcggtgcgcgtgtcccagcgataaaagagcggctaacaaaacgtcgcgagcggtcgcctggcggtgagcgcagtggtttcgttggctgcatcttgttggatgcagtcagaagccaggtcaaacgctgggtcgctcaagggcgca

SEQ ID NO: 31 - DNA, 711 bp, Xanthomonas campestris
  CDS (121)..(591)
ggtcggcgtgggctttgccgcgcagcaggttcccgccttgcccgtcgccgcctgggacgtgccggtggatgccatctgcaccgaacacgccaccttcctgaaagacgacaccgctcccgcatgactgcccgcaagcgctattggctgatgaagtccgagccggacacgttttccatcgatgacctggagcgcgttggcaccgagccgtggaacggcgtgcgcaactaccaggcgcgcaacttcatgcgcgatggcatgcaggtcggcgatggagtgttcttctaccactccaattgcaaggtgccgggcattgtcggcatcgccaaggtggccagcaccgcctacccggacgagacccagttcgatcggacatccgactatcacgaccccaaagccaagcgcgaagacccgcgctggatgctggtggacgtggcgttcgagcgcaaactcaaacgcaccatcgcgctggacgagatcaagcagcacgccgatgccctgggcgagggcttcccgctgatcgcacgcggcaatcgcctctccatcctgccggtgaccgccgcacagtggaagttgctgctggcgatggagtagccgggagtagagattcgggatttgagatttgcaaatcccggctcagcccagccgctcttgcgattccccaatcccgattccctaatcccgagctcttacccatgtccgaagcaaaacgcc

SEQ ID NO: 32 - DNA, 1245 bp, Xanthomonas campestris
  CDS (121)..(1125)
cctgccacagatcgcgcagcacgctgcaaggtgtaatcggttgcaggcattccgtgaaggaatgtctacgactttcgtcgctatactttcgttctcccccgcagctgccacaggaactacatgagcatcgaacagcttgccgaaaccgcccaggccatggtcgccccgggcaagggcatcatcgccatcgacgaatcgaccagcacgatcgccaagcgcttttccggcgtcggcatcgagaacaccgaagaaaaccgtcgtgcctaccgcgaactgctgctgaccacgcccaagctgagcgattacatctccggcgccatcctgttcgacgaaaccatccgtcagaagaccaaggacggcgtgccgttcgccaagtacatggccgaccacggcatcatcccgggcatcaaggtcgacaagggcgcgcagccgctggccggcatgccgggcgagttggtgaccgaaggtctggacggcctgcgtgcgcgtctggaggagtactacaccctgggcgcgcgcttcgccaagtggcgtgcggtcatcaacatcggcgaagacattccgtccggcacctgcatcgaggccaatgcgcatgcgctggcccgctatgccgcgctgtgccaggaacaaggcctggtgccgatggtggagccggaagtgatcatggatggcgaccacgacatcgagacctgctacgaagtcaccgaagccacgttgcgttcgctgttcggtgcgctgtatgagcagaacgtcgtgctcgaaggcaccatcctgaaggcctcgatggtcatctccggcaagggctgcgaagagcaggccagcgtcgaagaagtggccgagtccaccgtgatgtgcctgaagtcgaccgtgccggcaatcctgccgggcatcgtgttcctgtccggcggccagaccgacgagcagtccaccgcgcacctcaacgagatgcaccagctgggcaacctgccgtggccgctgagcttctcgtatggccgcgcgatgcagcaggccgcactgaagctgtggtccaaggacatgaccggcaactacgccaaggcgcagcagatcatttacgagcgcgccaaggaaaacggcttggctgcactgggcaagtggaagggctgatccgccgctgaggctgcatcacgcatacaagacgccggccgcgaggccggcgtcttgcgtttgggttcgccagtttggatggccggcagcttttgtcgcgacgtttgcatgtgcagcgtg

SEQ ID NO: 33 - DNA, 3705 bp, Xanthomonas campestris
  CDS (121)..(3585)
atcctcctggggtaacgttacctcgcaccgtggcaaccgaaacaggggcggcagttacgggcaacatcgcgtcagcgatgttcaccgttgcttggcagtagagcttgggagaggcctctaatgctgatggttcgcaaggcgttccgccgtggagcgctgtggctgttgtgtggttgcgtgggctggactgcagtggaggctgcacccgccaaggtcgatccgcccggtccttatgacgtgcgcgtgctcgcaggcggcgttggcttgagcaagacgctggccgctgatgcaccgatgctggcggccgacgcagactggagtgtgtccggttgggtgcgtcctgccatggacgtgcccggtaccgtgcgcattgcaggtctaggtgatgcgcgcggcgacgggcgcttcttcgtgatcgacaacggccgtcttggttttgcccaaggcgatagcgtgctgcgcagcaaacaggcgctgccggtagctacgtggacgcagattgcagcggtggcgcaaggtgcacggctgacgctgtatgccaatggacgccaggtggccagtggcacgcgcgtgtcacgtgcggtggcgccggcgctggtctttggcccacgtgtgccggccgatgccgcgtcgctgcacttcggtggcgacatcgccaatttcactgcgcgcgccggtgcgctcgatgcagcagctattgcgcagcaggcacggcacgcgccgaatgaaatattgcagcgcttcgaagatgcgtcgccaggctggcgcgtgcagaccaaacagatggctgggcagctcgaaccgcaaccggccgccacgctgccacgcagcgccgcaccgttctccgcaccggtggccaagcccgtcaccaatgcgcccgcattgcagccgctggatgcgctgtcatggcgcgtgggcggctggcagctggccgctgcgcctgacctcggcacgcgtggtggtgccgagctgtcgcgtggcgctgcaccttccagtagcgcatggcatgtggccaccgtgccgggcacggtactcaccacgctggtcgatcgcggtgtgtatccagatccggatatcggcctcaacaacatggccattcccgaatcgctgagccgccaggactggtggtaccgcagcaccttcgagctgcctgccagcacacagggcaagcgcctggaactggtgttcaacggcatcaattacgccggcgagatctgggtcaacggcgtgcaggtggggcgtacgcgtggcgcattcgcgcgtgggcggttcgatgtcagcagctacctcacgcccggccgcaatgcgattgccgtgcgtgtttcgccgccgccgcatccgggcattgcgcacgagcagtcgatgaccgccggcgttggcgagaacggtggcatgcaggcactggatgggcccaccttcattgccagcgaaggctgggactggatcccggcggtgcgcgaccgtaatgccgggctctggcaggatgtacagctgcatgccaccggcccgctcgcggtcggcgacacgcacgtggtcaccgccaaactggcgcccggccatcgccgcgccgagctggaaattgccgtgccattgcgcaacaccagcggcactgcggtgcagggcacagtgcagcttgccttcggtgatgtgcgtatccagcgtgaggtcaacgtgcctgcgggcggcaccacgctcacactcactgccgccgacacgccggagctggtggtgaccaatccacggctgtggtggccgaatgggtatggcgagccctcgctgtacaccttgcaggtcggtgtcgcagcgggcggtgtgcagtcggatctgcagcaactgcggttcggtattcgcgaggtcacttacgagctgtcgttgttcgacgaacgtggcgcactgcgtcgtgtgctggtggatctcaatcaggcccggcagcgtggcgagcgcatcgtcgatgtgcggcacgccgccattcgcccggtgcccggtggcaatgcgcaatcgctgtatcccggcgcactcgcgtcgccagcggtgacgcagctggatgacagtacgttggccccgcatctggtgctgcgcatcaacggtgtgcggattgcgatcaagggcggtaattggggtatggacgattggcgcaagcgcgtctcacgcgagcgcctggagccgtacttccgcctgcagcgcgatgcgcacttcaacgtggtgcgcaattgggtggggcagaacaccggagcgagcttcttcgacctggccgacgaatacggcatgttggtgttgaacgacttctggcaatccacgcagaactacaacatggagccggccgacgcggcgctgttcctggccaatgccgccgaggtcatcacgcgcttccgcaaccacacttcgattgtgctgtggttcggccgcaacgaaggcgttcccgcgccgatcctcaacgagggcctggacgcgctggtcgccgcgctcgatggcacacgctggtacaccgccagttccaacgaagtgaatctgcaaggcagcggcccgtacaactatcgcgagccggtggcgtacttcaacaagctggcacagggcttctcggtggaagtgggtacaccgtcgttctccaccctggagtccttcaaggcgtcggtacctgcggtgcaggatcaatggccgatcagtgacagctgggcgtatcacgactggcatcagtccggcaacggcgataccgccagcttcatgcgctcactcagcgacaagcttggcgcaccaaccagtctggacgatttcgaacgcaaggcgcagctgctcaattacgaaacctaccgcgccatcttcgaagggttcaacgcgcagttgtggacgaagaacagcggccggctgctgtggatgagccacccggcgtggcccagcaacatgtggcagatctacagccacgattacgacacccacgccgcgtactacggcgtgcgcaatgccgccgagccactgcatgtgcagatgaatctgcccggccacgaagtcgtggtggtcaacaacacgaccacagccgtgcgtggcctgcgtgtgcgggctgagacctatgcggccgacggccgcctgctgcaacagcgcgagcaggcggtggatgcagcagcggtggcggtgtctgcacccgtgcttcagctgacgccactgctggcgaacgcaaaggacctgggctttgtgcggctgcagttgctggagcgcgacggcacggtgcgctcgcgcaatttctactgggtggcgcgcgacgccgctgccatgcgctgcttggacgcattggcgccggtgccactggagctcaccacgcagctgcaggcaggcgatgcgccggtgttgagcgccactgtgcggaacccttcgcagcacgtggccctgggcgccaagctcaccttggtggatgcgcaaggccagcgcatcttgccggcctactacagcgacaactatctcaatcttgcgcctggcgagagccgcaccgtggagattcgcgggccggccgaggccagcttgcgcgccgcgcgcgtgcagttgcgcgggtggaatcttgcaccgtcttcgggcaccgccagcggcagtccctagcggcgtccttgcagttgctatgccgtgccccgcacggagctcgcactggcgcgttgaacgcgtcagacacaccgtgccgccccccggcggcaccattcgaaatggtcgtggagtgttgtc

SEQ ID NO: 34 - DNA, 1248 bp, Xanthomonas campestris
  CDS (121)..(1128)
ggcagcggcgtgagcgtgactgcgcgctggtgccgccagcttgcggagatacccgccggcgcctgggacagcctgcatgacggcgcgcaccccttcgtttcccatgccttcctcgaaggcatggagcgcgagggctgcctgcgcccggactggggctggcaggcactgcacctaacgttatgggacggcgacgccctggtcgccgctgccccgggctatctgaagaacaactcccatggcgagttcgtgttcgaccacgcctgggcgcatgcctatgcccgctacggccaggactacttccccaagtggttgtgcggggtgccgtactcgccggtgaccggcccccgcctgctgggcgacccgcgctggcacacgcacctgctcgacggcatgcgcaccctggtggaaagcgccggcctgtcgtctgcgcacgtcaatttctatcccgccaacgaggacgcggcattcaccgcagagtggctggagcgcaacgacctgcaataccactggcacaacgcgaccggctgggccgacttcgaggcctacctggccgcgatgaaccacaagcaccgcaagaacatccgccaggagcgtgccaaggtgcagcgcgccggcgtgcgttttcgtgtactgcatggtgatgaggccagcgcggacgagctgcaggccatgtacggcttctacctcaagaccttcgaggaatacggcaactcgcccgcgctgacgccgnaattcctgcagcacctggcgcagcacctgccgcggcagctggtgatgttcctggccgagctggacgggcaacccatcgccggggcgctctgcctgcgcggcggcgacaccctgtacgggcgctactggggcggcgaagcactgcctggcctgcattttgaaacctgctactaccagggcatcgagtattgcctgtgcgaaggcctgacccgcttcgaacccggcgcccaggggcaacacaagattgcgcgcggcttcctgcccaccctggtgcgcagccgccactggattgccgaccccggcttccgtgagccgctggcgcagtggtgcgccgatgagcgtgccgccgtggcccagcatgccgtcgagctggccgaacacagcccgttccgcgactgacaacatcgccgccgcgcagaccgcgccgctagactctgcgcatgtcgcgcaccctgccccatctgttgtctccggatcctgcgagccccttcccacccgccgagcacgccttgcgcgagc

SEQ ID NO: 35 - DNA, 996 bp, Xanthomonas campestris
  CDS (121)..(876)
ctaacgcattaactcgatgctcaagcgaggtgaagctgcgcgtgtgcaccggtgcgcactcacgcgcagcgaacccctcgcatccttagccttggcaacccctgtatcgaaggagaccgcatgcgaccgtccctcttgttgctgccgctggcgttttgcgccgcgtgctcgcgcattccaccaccggttacaccgccgggcgcggaagaaaccaccaagaccaatgtcctcgaagccggcgccaaggtcctgcaatccaatggaccggtgggcaaactcgatatctacctggttggctttcatccaatgaaggatgcgccggagcaacagatggaagcgcatcactactgccagcaggtcaaccaggatctggcgcagtgcgcgctctacgatggcaatacggccgaggccaatctcaccggcatcgagtacatcatttccgaaagcctgtttgcgcaactgcccgcgcaggaacgcgcgttctggcatccgcacaacggcgagatcctgtccggccagctctccgcacccaatctgccgctggttgccgaaaaggaactgatgcgcagcaagatgaacagctacggcaagacctggcacacctggcattcgcgcaagggcaccgcgccaggcgacacgctgccgctgggcgaaccgatgctggcgtggtcattcagtcgcgatggcgagatccagcagcccttgatcgagcagcgcgacaaggcggtgaccatttccacagccgagcgccgcgccgatcgcaaggacctcaccgagctggcgaaaccgcagcatggcgtggatgccctgcaccgccacttcccggaagcgacgccgatcccgggcgtggtggatgccgaggcaactgcgcagtaaacacgagtgccccgcctgcagttgcagcggggcaacggaatgccatcaatcactgaccaacttacggaacaaacgccgcgcgcacgcagccttcgtgcttgtgcttgaacatctcatagc

SEQ ID NO: 36 - DNA, 2148 bp, Xanthomonas campestris
  CDS (121)..(2028)
ccgcccccggtggtagccagtggcaggtgggctaccagcagccagtgatgggccgcaataccggtgctgccggcgggccattcgtggccgcctggtggcggttctagggcagggccgacaatgacggtcacctgcgaaatggacgcgctgggacgggcgccggatatcggccgcgaacgcggcttgttggggcgttcgctggtgtctgccggcgtcatcaccgacgagcaactacgcgccgcgcttgccttgcagcaacgctggaattcgcgcctgggcgatgtgatcctggcccagcgcggcgtgccggcgcagcggttctacgccatcgtggccgcgcacttcgggctgcagttcgtcgacctggtgcaacagccgccggaccccgagctgctcaccgccaccgatctggatgtgtatgcgcagcggctgatcctgccgtggcggcgcgaggatggtgtgctggtgctggccgtggccgacccggacccggcgctgttcgcctgggcgcgcgagcattacggtgcgcaagtccgcttcgtcggcactgccaagttcgacatcatctggagcctgcagcgctacgccgacgagcagctcaccgacaatgcgctgaacctgctggccgcacacgcgcctacctattccgcacgccaggtggtcacccgcgggcagaagttcaccttgtgggcgattgccgcggtgctggtggcggcaatcgccgcattccccatcatcacgctgatcaccatcaatgtgctggtagcgctgggctttctggcgaccttcggcctgaagttgctgctggtgtggttcggctcgcgccaccgcatcgacatcaaggtcaccgaagacgaagtggccgcgctgcgcgatgacgacctgccggtctataccgtgctggtgccgatgtacaaggagccggaagtcctgccgatcctggccaacgcgctgcgcaagctggattacccgatcagcaagctcgacgtgaaactggtgctggaagccgacgatttcgacaccatcgaagcggccaagaaactcggcctggaagcgttcttcgaaatcatccgggtaccgccgtcgcagcccaagaccaagcccaaggcctgcaactacgcgctgcatttcgcgcgcggcgaactgctcaccatctacgacgccgaagacaagcccgagccggatcagctcaagcgcgtggtggctgcgttccgcaaggcggaaaaagacgtggtgtgcatccaggcgcgcttgaactactacaacgccgacgaaaactggctgacgcggatgttcacgctggagtacacgctctggttcgacttctacctgccggcgctggaatacctgcgcattccgattccgctgggcggcacctccaatcacttccggctggacgtgttgcggcaggtgcgcgcgtgggacccgtacaacgtcaccgaagacgctgaccttggcgtgcggctgatccagaacggctaccgcgtcaacgtggtgaactccaccacgttcgaagaagccaacgtcagcattcccaactggatccggcagcgttcgcgctggctcaagggctacatgcagacctggctggtgcacatgcgcgacccggtgcatctgtaccgcagcaccgggttcaaggggttttggggctttcagttcttcattggcggtaattttttcatcgcactgggcgtgccggtgatgtggacgctgtgcctgatcagcatgctgagcggtgcacgcatatttgatgcgacctttccaccgtggctggccacgatctcgctggtcaatctgctgttggccaacgcgttcttcgtctacgtcacgctggtggccgcgttcaagcgcgactacttcaagctcgcgccgtatgcgctgacggtgccgttctattggttgctgcagtcgatcgcggcctacaagggtctgtggcagttgatccgcaatccgttctactgggagaagaccacgcatggcatcagcaagcattccgaacaagagcgccgcgccgcactcgaagagtgagccgggcgccgcgcgccgcgacagcgggttgccggcgttcctggtggcggtggtggcgttgtcgctgctgagccagccgttgctggtgcagtcgctgggcagcgcggcaccggcggtggc

SEQ ID NO: 37 - DNA, 1137 bp, Xanthomonas campestris
  CDS (121)..(1017)
cacggagaaccatgcgtgccgagctctggactcgacccccgcgtcgcaccaacgccccaccccgcccccccgccacagcgcgcgggcaaggtcgggcggttcggcagcctgcgtcgctgggtggattcgcaaaccgacgaaccgctggacgaagcccgcgcggaccgcatcgactggctgcgtgcgctgccgttcatcgccttgcacctggcctgctttgcggtgttctgggtcggtgtgtcgtggttcgcagtgggcatggccgtggcgctgtacgcgctgcgcatgttcgcgctgaccgggttctatcaccgctatttctcgcaccgcgcattcaagacctcgcgcgtggtgcagttcgtgttcgccgcgatcgccgccacctgcgtgcaacgtgggccgctgtggtgggccgcacaccatcgcaaccatcaccgccataccgacaccggccgtgacccgcattcgccggcacagcacggcttctggtggagccataccggctggttcctgaccccgcgcaacttccgcaccgattgggaagtgattccggatctgcgccgctttaccgagctgcgatttctggaccgcttcgacatcgtgatgccggtgctgctggcgctgggcctgtacctgctcggtgactggctggcggctgcagcgcctggcctgcagaccaatggcgcgcagttgctggtgtggggcttcgtgctgtccacggtggcgttgttccatgccaccttcaccatcaattcgctggcgcaccgcttcggcagccggcgcttcgacacccgcgacgacagccgcaacaactggctgctggcgctgctcaccttcggcgaaggctggcacaacaaccatcatttctttcccggctcggcgcgccagggcgtgcgttggtgggaatacgacatgacctggtacgggctcacggtgatgtcgtgggtgggcctggtgtgggatctcaaaccgatgcccgccatgctgacgcgacgcgccggacgggtggcgcgatgagcggtggcaggatcgcggtggtcggcagtggaatcgccggcctgggcgccgcatggctgctgtcgcggcgttacgaagtgacgctgttcgaggccgccgattacctcggcggccacacgc

SEQ ID NO: 38 - DNA, 1491 bp, Xanthomonas campestris
  CDS (121)..(1371)
gcgttggtgggaatacgacatgacctggtacgggctcacggtgatgtcgtgggtgggcctggtgtgggatctcaaaccgatgcccgccatgctgacgcgacgcgccggacgggtggcgcgatgagcggtggcaggatcgcggtggtcggcagtggaatcgccggcctgggcgccgcatggctgctgtcgcggcgttacgaagtgacgctgttcgaggccgccgattacctcggcggccacacgcacacccacgacatccagttggagggccagcgttacgcggtggacagcggcttcatcgtgttcaacccgcagcactaccccttgctcacccggttgttcgccgaaatcgacgtggcgtcgcagtccaccaccatgagcttttcggtgcacgatgcgcgcagcgggctcgaatacaacgccggctcgctcggcgggctgttctgccagcgccgcaacctgctcagcccgcgcttctggggcatgctggccgacctgcggcgtttttaccggcaggcgcctgcggtactgaccagcgacgagcgtttcagcacgttgggcgaatacctgcaccggcacggctattcggacacgtttcgcgaccagcatctggtgccgatggcctcggcgctgtggtcctcgccctcgcaacgcattctcgaatttccgatgggccagttgatcggcttcatggccaatcaccacatgctgcagatcagcggacggccgcagtggcaggtggtgcgcggtggttcgtcgagctatgtgcgcgcgttgcggcggcgttggcaggtgcaggaacgcctgtccacgccggtgcacgccgtccgccgggctggcaacggcgtggtcgtgacctcgttgcgcggcgacgagcatttcgacgaggtggtactcgcctgccatgccgacgatgcattggccttgctcagcgatgccacgcctgccgagcagcagatcctgggcgccatcacctaccagaacaacgacaccgtgctgcacaccgatgccagcgtgctgccgcgcgaccgccgcgcctgggcggcctggaatgcgcatgtgccggccgacccgcaggcgccgtgcacggtgagctactggatgaacgcgctgcagtcgatcaacgcaccgcagccgttcatcgtcagcctcaatcgcgaccaggacatcgcaccggagaaagtgctgcgccgcatgcgctaccgccacccggtgcagaacgccgccgcactcgatgcgcaggcgcgcaaggcgcagatccagggccagcggcacacctggtttgccggcgcggcgtggggcttcggcttccacgaagatggcctgcgcagcggcgtggacgtggcgcatgcactgggagtgccgtggtgacccagctgctgcgcgaagcgatcaccgatgccgttccactgccgcccaccgagggcggtggtaccggacggctgcacggcccgttgcgcagcgccatctataccggctgggtgcggcacc

SEQ ID NO: 39 - DNA, 1023 bp, Xanthomonas campestris
  CDS (121)..(903)
accgcagcgcaacgcgctgctggcgctggcgattggctggggcgcggcctgtggcctgctctcgctgtatgcccgccgcctggcacgccccgcgtttgagcccatcactggagacccgcaatgactgtctggccgctgctgcatgtcggcgtgttcgccgccgtggtgatggtgttgggttggctgtggcaacgccgcagtggcaacgccggcccggtggacgtcttgtgggcgggctgcctggcggttgccgcgccctactgtgcgtggatgtccgatggcgcgccgctgccacgcgtgctggtggcggtgctcggcggcgtgtggggcgcacggcttgcctggcacctgggcgtgcgcgtgttcggcgacccccacgaggatggccgctaccgcgccctgcgtgagcactggaacggcgatcagcgcaagttcctgggctttttcctggcccaggcggtggtggtggtgctgttctcggtgccgttcctggccgcggccagcaacccgaacccggcatggagcgtgtggagcagcctggcgattgtggtgtggttgatcgccgtcggcggcgaggcgctggccgaccggcagttgtctgcgcacaaggccaacccggccaatcgcggcaagacgtgtcgcaccgggctctggcgctattcgcggcatcccaattacttcttcgaattcgtgcattggtttgcgtacctggccctggcggtcggtgcggggccgtggccggtggcgctgtgtgcgctcggcccggtggtgatgttcgtgttcctgtatcgcttcaccggcatcccctataccgagcagcaggcactgcgctcgcgtggcgaggactacgcccagtaccaacgcagcaccagcgcgtttttcccgcttcccccttcgtcctgattcgcctggagagtcgcatgtctactgtcaccatcccgccctccgcgcatcccgaagaaggctttgccacccgcttggccgaatccggcgtgctgcccgatgccgcgttgcgttacggca

SEQ ID NO: 40 - DNA, 1032 bp, Xanthomonas campestris
  CDS (121)..(912)
ctggcgcgtggatgccaacaacctgttgttcgccaatctctatctgcaggtggaggtcaaaaacgcggcctccggcagcaacctcaatgtgcagttcgtccacgtgttccagcgccggtgatgccggcaacttcggagaaggcaatgcgcttgttgaacaagaccatcgtggtgaccggcgtggcatcgggcatcggtgcggaagtggcgcgcttggcgcgctttgaaggcgcaaccgtgatcggcatcgatcgcacgccggtgcatatgagcctgcacggcttccatcaggccgacttgggcgatcccgctgcgatcgatgcgctggtgcaggcactgccggaacggatcgacgcgctggccaatgtggccggcgtccccggcaccgcgccgctggaggcggtggcgcgggtcaactacctgggcctgcgtcacctcagccacgcgctgctgccgcgcatcagccgtggcggcgccatcatcaacgtcgcctcgattctcggcgccgaatggccgcagcggctggacctgcacaagcagctggccgccgctgccagttttgccgacggcagcgcttggctggccgcgcacccggttgcgcacgacacctgctatcagtatttcaaggaagccctgatcgtgtggaccttgctgcaatcgtggccgtggttcgccgatcacggcgtacgcgtcaacaacgtctcgcccgggccggtgttcaccccgatcctgggcgactttgccgcgatgcttggcgccgagcgtgtgcaggaagacgccaaacgcatgcaacgccccgcgtatgccgacgaggtggccgaggcgatcgtgttcctggcatcggacgccgcgcgctggatcaatggcatcaccttgccggtcgatggcggcctggcatccaccactttgtaagtccccttggacccccaaccagcaggaggctcgaccatgcatgcgctccccagcattgcgacacccacgaccgccacaccggcgtggcacggctgtatcttcaatggcgactgggttccc

SEQ ID NO: 41 - DNA, 1053 bp, Xanthomonas campestris
  CDS (121)..(933)
tgcactgatcaggaaagatcgtgaacaggttctaagccgcgcgtcgcgcaggccgggtgcctgcgcgaatgggcaaacgctgcaatccgccttacaaatgcgacgcgtacaattccggggatgaagaccttctccccgttgttgttgacgctctccgtgtgtgcagccgcgctgcagcccaccgccgcactggcctggggcccgcagggccaccggctggtggcgcgtgttgccgaaaccgaattgacgccgcaggcgcgcgcgcaggtcagtcagttgctggcaggcgaagccgaccccagcctggccggtgtggccacctgggccgatgagttgcgcgccaatgatccggatctgggcaagcgctcgggcccctggcattacgtcaatctcggcgaacacgactgcggctatgtaccgccgcgcgattgcccggatggcaactgcgtgatcgctgcgctggagcagcagaccgccgtgctggccgatcgcaaccaaccgttggccgcacgcaggcaggcgctgaagttcgtggtgcattttgttggcgacatccatcagccaatgcacgccggctatgcgcacgacaagggcggcaatgacttccagctacaggttgccggcaagggcagcaatctgcacgcgctgtgggacagcggcatgctcaatgaccgccacctcagcgacgatgcctatctgaaagtgctgctggccttgccggcaaccgcgcccacctcgccggcgctgccgccgccgggcgcggcctgggcacaggcgtcgtgccgcatcgcgatcacgcccggcgtgtatcccgacacgcatgtactgccaccaacctatatcgccacctatcgcccgattgccgagacccagctgcgtgtcgcgggcgaccggctggccgcgctgctcaatgcggcgctgcgcagcccctgagacggagataccgatgcagccgcaggtccttgcgttcttccatgccgacagcaacacctttacctacgtggttgccgatacgcgcaccggggcggcggcggtgatcgacccggcgctgga

SEQ ID NO: 42 - DNA, 3000 bp, Xanthomonas campestris
  CDS (121)..(2880)
tggcctgggtctgctgatccagtggtggatgatccgccagcacggcgagaagccgagcaagatcatccaggccaacgccaagtaagtggaacaaggcccgccgcaaggcgggctttgtacatgtgcccccgcagtgcaccaccgcacgtgctgaccattgccaaggatgcgttcgtgatgtctttgccgctgcaccgcctgttgctgtgcgccagttgtctggggctgttgctgctggctggctgcggcaaaccgcaggacgggcgcgatgggcaggaggctgccaagccggccgcatccgccacgccggccgcgcaaaacgatcccgctgccgaagatgcgcgcgtcctgatcgcgcagttgcgcgaacagctggaccggcatcgccgcatcatcgtgttgctggccgatgaggccgcgcaatcgccgcgcgaccgtgccacctccagcagcgtgggccagcagttgttccatgagaacctggagcagcgcgagcgtattgctgcacgattcgagaccgtgctggcctccaaggcgtccacccgctttgccacccttgccacggtgctggacgacgtcgagtcggcgcccgatctctacgatgccgatcgtctggcctttcgcgaagtgctgcgcgatttgcacgcgcgtattggccgcgattcggcattgccggcggtgaaactgcaccagcgcatcggcgaagacctggaggcgctggatgaaatcgagcgcacctacaacaccgagctgacccagatcttcagccgcttcgatcgcagccgcgccatcacgcccaagcgcgagaaatgggacgactacgtggcgcatctgcgcacgctgtacacgcgcgaaaagatcctgcgcgaccacggcgtggtggagccgtatccgttctccgcgcgcgacagcgagagcgagatcttcggccgcgacctgccgcccaagaccgtggtgctgaccttcgacgatggcccgcacaaggcctataccgaagagatcgtggccatcctcaaacgctacgacgtgcccggcgtgttcttcgaagtggggcgcaatctgggcaacctggatgcgcaaggcaagccgaccctggcgccgctggccaagatcagccacagcctgatggaagaaggctatgcggtgggcaatcacagcctcacccatgcacagctatccaagaccaccggtgcggccttgcgcagccaggtgctggataccgacacgctgttgcgtgcggtggacgacaagcgcgcgccgctgtttcggtttccgtatggcgcacgcaatgccgaagggctgcaattgctcggcgaagcgggcctgaaatcgatcatgtggaacatcgattcaatggactgggccgacccggtgccggaatcgatcgtgcagcgcgtgctcgaccaggtgaacaaggaacagcgcggcatcgtgctgttccacgatatccacgaccgcgccgtaaaagcgttgccgcagatcctggatcgcctgattgccgatggctaccagtttgccggctggaacgggcgcgatttctcggtggcacgcgcacccagcgcgacagcggccgcaacggtgaccaccggttatgccaagtcgtgggcgatcgtgatcggcatcgacgactacgcgcgctggcccaagctcgaatacgccgccaacgatgcgcaggcgattgccaacaccttgaccggcccgttggggtttccgtcgtcgcaggtgatcgtgctcaagaatgccgaagccacccgcaacaacatcctggccgcgttccacgaccggctcggcgatggacgcgcgcagaccaacgaccgcgtgttcgtgttcttcgccgggcatggcgccacgcgccggctcgcctccgggcgcgacctgggctacatcatcccggtggattcggacccggagcatctggccaatgacgcgatcgccatgagcgacctgcagaacatcgccgaaagcctgcaggccaagcacgtgctgttcgtgatggatgcctgctacagcggtttgggattgacccgtggcggcccaagttccagcgcctacctgcgtgagaacgcccgccgtatcgcgcgccagatgctcaccgccggcggcgccgatcaacaggtggccgacagtggcccgaacgggcattcggtgttcacctgggtggtgttgcaggcgctggccggcaagggcgatctcaatggcgatggcttgatcaccggaaccgagctggccgcgtatgtcgcgccggccgtgtctgcggtgtcggcacagacgccggcattcggcagcctgccgggatcgcagggcggtgagtttgtgttccaggtgccgagtggcgaagaattcctcaccgccgataccgcgcagttgtcggcggccgcgatcgcgctcaatggccgcgtggatgcggtgcgcccggctgcggtcagtgccggcgtaccggcaaacgcggcgccggtgacggtcaagaccttgcagggcggcgaagccacgttggtgctgccgaccgcggtgaagagcagcgaccggcagcgtgcgcaattggccaacgaacgtggtttgcagctgtacaaggaaaaacgctacgccgatgccgccgagcagttcgccgaagccctgaagctacggccggactttgcattggctgccaataacctggggtttgtgtactaccgccaggagcgctatgccgaagccgcacgctggctggaaaacaccttgaagatcgatccctcgcgcgcggtggcctatctcaacctcggcgatgcctatgccaaggcaggcgatcgcgagaaggcacgcaaggcctacaccacctatctcgccctgcagccgcaaggtgccggtgccgagcaggcacgcgcacagttgcagaccctgtgatttctcttcgttctctcatcgcatgaatgctttgccttccaccatcgttgccatcgccactgctgccggaactggcggtatcggtatcgtgcggctgtccggcccgcagtctgtacagat

SEQ ID NO: 43 - DNA, 2211 bp, Xanthomonas campestris
  CDS (121)..(2091)
gcgcggcgggtccagcggccatccgccacagaccgcacactgtcacgccgcgccgcagctggacggaaaccgctgcaggcgctagcgtgatgcggattttttgcctcaggggaatgcccaatgcgccatgacgcgcagccgcgccagctcaccttccgtgccgtcgtgctggccatcgtgctggcggtggtgttgtcggccgccaatgcctatctcggcctgttcgccgggttgaccattgccaccgccattccggcggcggttgtctcgatgggcgtgctgcgcctgctcggtggcggcaccatcctggaaaacaacatcgtccagaccggcgcttcggccggctcgtcgatcgcggcgggggtgatcttcaccatcccggcgctggtgatcatgggctactggccggacttcaagtactggtgggtgctcggcatcgccggcatgggcggcctgctcggcgtgctgttctcggtgccgttgcggcgctcgatgatcgtcgaagacccgctgccgttccccgaaggcaaggccgccgccgaagtgctcaaggccggcgagaaccccggcccgggcctgaagatcctggccatctccggtgccatcggcgcgctggtcaaactgggcgcggccagcggcctgcgcgtgatccccgacacctgggcgcaggccacctacctgggcagcagccgcctggttggctatctgggcaccaacctgtcgccggccttgctcggcgtgggctacatcgtcggcttgaacgtcggcatcgtggtgctgtccggctcgatcctgtcctggcacatcgccatcccgctgtaccagcagttcttcatgggcagcgacccggcgctggcacagagcctggtcaatgcgccggcagccgatgccgcattcggtatctggggcgcgaagatccgctacctcggtgtgggcgcaatgctgatcggcggcgtgtggacgctgttctcgctgcgcaagtcgctgttctccggcatcaagagcggttttgccgctgcgcgtaaaagcggtggcggcgtggttgccgaaaccgagcgcgatctgccgatgaagtggatgctggtggcgctggtgctgtgcaccttgccgctgctgggcctgtaccaggccatcgtgcagcagtggcacgtgtctatcccgatgaccatcatcatgatcgtggccggcttcctgttcgtgtcggtgtccggctacctggccggcctgatcggctcgtccaacaatccggtgtcgggcatcaccatttccaccatcctgttcgcctcggcggtgctggtgttgctgctgggcaagagcgggctggtgccggtgggcatcggcgccgccccgttgggcgcggtggccgcgatcatgatcggcgcggtggtgtgctgcgcggcggcggtgggcggcgacaacctgcaggatctcaaggccggttatctggtcggcgccacgccgtggaagcagcagctgatgctgggcatcggtgcgttctcgtgcgcattgatcatggcgccggtgctgaacctgctcgcccaggcctacggcatcggcccggccacgccgcagcatcccaactcgctgggcgcgccgcaggccaccttgatggcttcggtggccaaaggcctgtttggcggccagttgccgtggagcatgatcgccatcggtgccggcgtgggcgcggccatcattgcgctcgacgaatggttgaagaagaccggcaagcgcttccgcgtgccggtcctggccgcggcgatcggtatctacctgccgctggaactgatggtgccgatcttcctcggtggcctgatcgcgcatctggtggagcgcttccacaccatccgcgccgacgacgaagacggccgtgaccgcgtgcatcgtccgggcgtgctgtttgccgccggcctgatcaccggcgaagccttgatgggcatcgccattgcggtgccgatcgtgctgtccagccgcgccgatgtgctggcactgccggctgctttgcagctcaatcaatgggttggcctggcgatcctggccctggtgggctggctgctctatcgcgtcggcaagcgcggcgaaaaagccgcctgagtcagcgacacacacacggcgcatgcgatgcatgcgccgtcctggcaggcgcgcaaggcgacctgcctgcagcgcatccctctgccctctgcaccatccgcacgcaccgccgccgcagac

SEQ ID NO: 44 - DNA, 1056 bp, Xanthomonas campestris
  CDS (121)..(936)
gattcgtaaagcagcatgggtgcggcgcccgcatctgtattggtctgtttgtagcgtatctgaatctgtcctggtcgcgcatcgcgccgcagactggccgtgccgggcgacccgcgcggcatggtcttctggcgaggttgcaacgtgctgctttccctgttcgtggtcgatgcgtttacgtgtgtgcgtttcaaaggcaaccctgcggcggtggtgccgttgcaggcatggttaccggtgccggtgatgcaggcgattgctgccgaaaacaatctgtcggaaaccgcgttctttgtgcgtgatcacgccggcttcttcgacatccgttggttctcgccgttgaaggaagtcgggttctgcggccatgccacgctggcaagtgcgtacgtcattgcgcgccagcagctggcaccgtttccgttgcgcttgcgcgccccggcggtcggtgagatcggtgtggagcaggtggacgaagcgacctttgagatgtgctttcccaatcagctgccgaccctggtcacgcaggtcccggcgccactgctggccgcgttgccgatcgcaccgcaggcggtgtatgccaacgcacaggcctacatcgcggtgtacgcggacgagcagcaggtgcgtgccgtggtgcccgacctggcgcagatgctggcattggcaccacgcgatgtggccatcaccgcaccgggagccacgcatgacctggtgtcgcgctatttctggccggccaatggcggagcggaggaccctgtgaccggttcgatccatgcagcgctggcgccgttgtgggcacgcacgcttggaaagaccgatctgcgcgcgctgcaggcctcggcacgtggcggtgagttggcgtgccgtgtcacgccggcgcatgtctacgtgcgtggcagcgccgtgcagtatctgcgtggggagatcacgctatgagcgttgcagctgcaccaccggtggcggcgtcgcgccgcgcaatctggcagctgctgttggccgaggtgttgatcggttcggttggcgtcttcgtgcacgaaagtggtcaggacccggtga

SEQ ID NO: 45 - DNA, 1524 bp, Xanthomonas campestris
  CDS (121)..(1404)
gcgtgtgaattccatccttgagcgtgtgcagccgtcgccaccggctgctccatccagaggcgatcttctgcgtgttctcgaacaacaccgcagcgttgattttgcgcatccacttgcgtgatgtcctggcccgttatggagtccgccatgcagtccatgtgggagttggatgtcagtccgacacgtttcaaagcgcatttttcgctcgatccgggcgtggcatgtctcaaccacggcatgctcggcgcctgcccgatttcagtgcagcagcggcagagtaccttgcgtgcacaactggagcggcaaccggctgcattcgtgctccgcgcgttacccgctctgctcgatacagcgcggaattgcttggccgaggtgatcggtgccgacccgcaagatctgctgctgttgcccaatgtcaccaccgccatgagcgcggtgctgcgctcacgcatattcgcaccgggcgaccagattctgaccaccgaccacgcctatctttcctgccgcaatttgctcgacttcattgcgcgcagcaccggcgccgaggtgatggtcgcgcctgtcaaggtaccggtgcaccatcccgatgcgatcgtcgatgccgtgcttgagcgggtgacagcgcgcacccgcctggcggtgctcgaccatgtcaccagcccgaccgcgatcgtgttcccgatcgcacgcctggtcgagcgccttgcagcgctcggcatcgacacgcttgtggatggcgcgcacgccccgggtatgctgccactcgacgttatcgccatcggtgcggcgtattacgccggggattgccacaagtggctgtgcacgccgcgcggtgcgggttttctgcatgtgcgccgcgatcgccaggatggtttgcatccgccggtgatcagtcgcgggtatggcgatgcaacgccgggccggccgcggttgcatctggaattcgattggctgggcacggcagacccgactgccttgctgtgcattcccgctgcgattgattttctggccacgctgctgcctggtgggttgcccgcggtgttcgcacgcaaccatgcactggtgacgcaggccgctgcgcggttggcgcaggccttgccgctcgcgcgccttgcacccgatgcgatggtgggcagcatggtggcattccagctgccggatgccgcgagcgatgcgcccgacgatgccgcggcgtcgttgcaacgttggctttacgacgcacatcgcatcgatgtggcggtgacgccctggccgcatcgaactaaccgcacattgcgcatttcggcgcaggtgtacaacgcagcggaggatttcatgcagctggatgcgccacttgcgcagtgcttggcgcctgatgacaggcaatcgaacaatcaatgccgcaccactgaggtcatgcgatgaaacagcccggcttcgaagacaagcatgtgttgatcaccggcggcgccggtggcatcggcatcgagctgatacgcgtgttcgcaaacgcatcctgccgggtgacattcactcaccgcccag

SEQ ID NO: 46 - DNA, 1143 bp, Xanthomonas campestris
  CDS (121)..(1023)
cagagcatccagtgcatcttgctctttccccagtgtggacacgcgcgacggcatctttccatgccccagccggtacctgtgccgcgcagaatcacttgaaccgaattggatacgttcttcatgacttaccccggctattccttcacccctaagcgcctggatgtgcgtcccggcattgcgatgtcgtatctggacgaaggtccgcgcgatggcgaggtggtggtcatgctgcacggcaatccgtcgtggagctatctgtggcggcatctagtgagcggtctgtctgatcgctaccgctgcatcgtgcccgaccacatcggcatgggcctgtccgacaagcccgacgacgcgcccgatgcgcagccgcgttacgactacacgttgcaatcgcgcgtggacgatctggaccgcctgctgcagcacctgggcatcaccgggccgatcacgctcgcggtgcacgattggggcggcatgatcggttttggctgggctctttcgcaccacgagcaggtcaagcgcctggtgatcaccaacaccgccgcgttcccgctaccgccggaaaagccgatgccatggcagatcgcgatgggccggcattggcggttgggcgagtggttcatccgcaccttcaatgcgttctcctccggcgcctcgtggttgggcgtgtcgcggcggatgccggcggctgtgcgccgcgcctatgtcgccccgtacgacaactggaagaaccgcatttccacgatccgcttcatgcaggacatcccgctgtcgtcggccgatcaggcctggtcgctgctggagcgctcggcgcaagcgttgccgtcgtttgccgaccgcccggccttcatcgcctggggtttgcgcgacatctgtttcgacaagcactttctggccggtttccgccgcgcactgccgcaggccgaggtgatggcgttcgacgatgccaaccactacgtgctcgaagacaagcacgaggtgctggtgccggcaatccgcgcgttcctggaacgcaacccgctgtaggacaagtttcgcatccgtgcgcgcgcgcgccgaagagctgcgcgcgccgtgtgcgaagtggatgcaccgggcaatgcgtgcatcgcgcaggtccggattggatcgcactgcggctgcgcc

SEQ ID NO: 47 - DNA, 1356 bp, Xanthomonas campestris
  CDS (121)..(1236)
tgcactgtgtcttgattttggtttcatttgcaactacaaacggagggtggcctgcgcattctcgcgggtcttctgccaccccgcatagccccgcgggcgccctagcgtggagaaacgacgatgagcgcacaaccgaacaccactgcaacgcgcccggacccgggcatgcaggtcaccaccttcgaaaatccgatgggcatcgacgggttcgaattcgtcgaattcgccgcccccgccggccaggccgcgcagttgcacgactacttccgcaagatgggcttcaccgcggtgctgcgccatcgcagccgcccgatcaccgtgtatcgccagggcggggtgaactttctgctcaacgaagacccggattcgttcgcggccgatttcgccgccgcccacggcccgtgcgcctgcggttttgcgatccgcttccgcaccccggccgacaccgtgctgcagaccgtgctcggcaacggcggcgaagccgtgcagaacaagcccgacacgcgcgcggtgccggcgccggtggtcaagggcattggcgattgcatgctgtacctggtggaccgctacggcgaggcggggagcatctacgacgccgactacgaggcaatcgaaggcgccgaccagcacccggccggcttcgggctcaccttcatcgaccacctgacccacaacctgtacttcggcaacatgcagcagtggtcggattactacgagcggctgttcaacttccgcgagatccgctacttcgacatcaagggcgccaagaccggtttggtgtccaaggcgatgactgccccggacggcattgtgcgcattccgctcaatgagtcttccgacccgaagagccagatcaacgaatacctggacgcgtatcagggcgaaggcattcagcacatcgcctgcttcaccgacgacatctacacctcggtggaacggatgcgcgctgccggcgtcacattcctggacacgccggacacctatttcgacgtggtggacctgcgcattcccgatcatggtgaagacgtcgagcgcctgcgtcgcaacaagatcctgatcgacgccgacgtggacaccaaacagcgcaagttgctgcagatcttcaccagcaactgcatcggcccgatcttcttcgagatcatccagcgcaagggcaatgaaggcttcggcgaaggcaacttccaggccttgttcgaaagcatcgagcgcgatcagatgaagcgtggggtgctttgatagccgggatgggtgattcgctactcgggatttgtggttgctgccattaattgcttcggtgcggttttgagtggtgcagattgttgcggtgccgcgcctggctgcccgtgatgtcggatg

SEQ ID NO: 48 - DNA, 1545 bp, Xanthomonas campestris
  CDS (121)..(1425)
cccggtgggagaggggcttgatccgccaggcttgagctctttttcggtaagagcggcttgatccaactcgatcccacccttctgctgtcgtggagggcgggtcaacatccggacgcacccatgcacaacgaccagcgttacatgaccggcttcggcaatgaattcgccagcgaggcagtagccgataccttgccggttggccagaactcgccgcagcgtgtggcgcacgggctgtatgccgagcagttgtcgggtactgcattcaccgcgccgcgcggcgaaaatcggcgcagttggctgtaccgcatgcgtcctgctgcggtgcacgggacgttctcgctgatcgagcagtcgcagttccacaacgatttcggccacggaccggtgccgccggaccagttgcgctggagcccgctgccgttgccgcagacacccaccgatttcatcgatggcctgtacaccatggccgggaatggtagccccgaagcaatgaatggcgtggccgtgcatctgtatgcggccaatgcctccatgcaggatcgcttcttctataacgccgacggcgagctgctgctggtgccgcaactcgggcgcctgcgcgtgcacaccgagctcggcatgctcgagctggagccgcagcagatcggggtgattccgcgcggcgtgcgcttccgggtcgagctacgcgacggcaccgcacgtggctatgtctgcgagaacttcggtggtttgctgcacctgcccgacctggggccgattggcagtaatggcctggcgaatccgcgcgacttcgaaaccccgtgtgcggccttcgagcaacgcgaaggccgctttgaactggtggccaaattccagggccacctgtggcgtgcggacatcggccattcgccactggacgtggtggcctggcacggcaactacgcgccgtaccgctacgacctgcgccgcttcaacaccatcggttcgatcagcttcgatcatccggacccgagcatttttacggtgctgacctcgcccagcgacacgcacggcacggccaacatggatttcgctatcttcccgccgcgctggctggtggcgcagcacaccttccgcccaccgtggttccaccgcaatgtggccagcgagttcatggggctggtgcacggcgtgtacgacgccaaggccgacggcttcgcgccgggcggtgcgtcgctgcacaactgcatgagcggccacggcccggatgccgccaccttcgacaaggcttcgcaagccgacctgtcgcggccggacgtcatcaccgagacgatggccttcatgttcgaaacccgcgccgtgctgcgccccaccgcgcaatcgctgcacgcaccgcatcggcagggcgattaccagcagtgctgggcggggttgcgcaatgcatttcaggcgccgccggctagctgagtgcggtagcgacggcgcaatagtgtggtgttgagcggtgcctcataacgctgcacgctgttgcgcatcaaccctaagtctcgcaacagcacgagcgtccgaagcaccgcgatgaccgtt

SEQ ID NO: 49 - DNA, 1137 bp, Xanthomonas campestris
  CDS (121)..(1017)
gatccgatcactgcgatcgggcccgtctgcgccccggttgcgctcaccaccgattcattgccccgcgactaacgtcgcgccattgatttatctttgacggattgccctttggagcttgtcatgccccgtttgtcgcgcctggccctgttgttgtcgctgaccttcgccgccggccatgccgtggcgggcccggaagaggaccagcgcgcgcgtaatgcggtgcgggtgctcaacgagatcatgaacatccccgagcaggccatccccgacaagctgctcgatgaggcacgcgcgatcgtggtcatccccgacacgctcaaggccggcctggtgattggcggccgtcgcggccacggcctgatgtcgatgaagaacgccgacggcagctggtcgcaaccggtgttcgtcaagctcaccggcggcagcatcggcttccaggccggcgtgcagtcttccgatgtggtgctggtgttccgcaacgaccgcagcctggacaacatcgtcaacggcaaattcaccctgggcgccgatgccggcgtggccgcaggcccggtgggccgcaacgccgccgctgccaccgacggccagctcaaggccgaaatctggtcgtggtcgcgcgcgcgcggcctgtttgccggcgtggcgctggatggcgcggtgctgcagatcgacgacgccgccgacctcaacgcctacggcggcggcgccaccccgcgcatgattttcgaaggccgcaccaacgagcgcccgtccaccgacgtgatcgccttccgcgaccgtctggaagaggtcacctacaccgcgcgcaccaaccgcagcagcgatggcgacacgccgccgcctgcgccgcgcccggtcagccagtcacccgcgcaggccccggcgacgccaccgccgccgggcaacgaggccagcaccatgccgatccagccctcagccacgccgccgcagcagcaaggctttcagccggtctccgatggtgaaatccgcaccgaatcactagacggcaaccgctgaccggctgtcatccccctgcgcggtgcggtggggtatcctgagtctcctattcttcaagcgagcgagcggatcatgggcagtttcagcatttggcactggctgatcgtgctggtgatcgtg

SEQ ID NO: 50 - DNA, 1299 bp, Xanthomonas campestris
  CDS (121)..(1179)
gacgtcgcaataattttgtatggcgtcgctgcaccacgcggatcagcctgcttttgcagggtcacggcgcacgcgcgtgccgatacactggccagtcatcgcaatcaggcagagacgtccatgagctctttggcgacttcgttccgcgcagccgtgttcgctgccggggtcacatgtattcctgcgcacgttggcgccacacccgatggcaaccgcacatcggctgaagccgcacaggcggtgcagcctgcgcagtggcgcacggtggccacgcacaacacgccgcacgcgcgccatgagaacagcatggtcgccattgatgatcgtctgtacctgctgggtgggcgcgacgaccggccgctggagatcttcgataccaccacgcagcggtggtcgcagggcgcgacgccgccgctgatggtcaatcatgcccaggccgtggtgtcgtccggcaagctctacgtggtgggtggcttttcaggcaactatcccgaagaagcctcgctcaccaacatgctgatctacgacccgaagaccgaccattggcaggtcgggccggagattcccacgcagcgccgtcgcggtgccgccggcacggtggagcatgccggtgtgttgtatctggtcggcggcaatacgcgtggccacatgagcggctacgtgccctggctggatgctttcgacacccggacacaacaatggacgcagctgcccgacgcaccgcatgcgcgcgatcacttccatgcggtggtactcgacggcaagctgtatgccgccggcgggcgtcgttccgcgcatgaaagcggcaataccctggcgcagacaatcagtgaggtcgatgtgtatgacattgcacagcgcagctggacagttgcacccgccatgctgccgacgccacgtgcgggcaccgccgcgatcgcgcgcgatggccggctgctcgtcatgggcggcgagagcacgcgtcaggtgaaggcgcacgaagaggtggaggcctacgacccgcgtacggcgcgctggacgacactgccgacgttgccgcggggccgccacggcacgcaggccgccgcggtgaagggcgatgtctatctcgctgcgggtagcgccaaccggggcggtgggccggagctggaggatgtgctggtgttggcgaaggcgcaggggacgcagtcactggactagccagtgcgctgcgggcgtgcctgcatcaagcgcctgcgcatgccgtacatcggcgctgccctgtcgcggccaaggccgctcctacggtggagcggctatcgcgagccttcggtgcgtggt

SEQ ID NO: 51 - DNA, 1108 bp, Xanthomonas campestris
  CDS (77)..(988)
gaaatcaggcctggggttcgccatgatttaatgcccgcatcgttatggtgaacgctgttttcgaggagttccccccatgcgtaaaggtgctgcactgatggtgggcgtgaccggcatttccggatacaacttcgccaatgtgttgctcgccgacggctggaccgtctatggcctggcgcgccgcccgctgtcgcacgaaggcgtgatcccggtagcggccgatctgctcgatgcggaggcaaccaaaactgcgctgcaggggctaccgatcacgcacgtgttcttctgcacctggacgcgccgcgataccgagcgcgagaacgtcgaagccaacggcgcgatgatgcgacacctgtgcgatggcttgagcgaggcgccgcttcagcacatggcgctggtcaccggcaccaagcactacctgggtgctttcgagaactacggcagtggcaaggcggaaaccccgttccgcgaaagcgaaccgcgccagcctggcgagaacttctactacaccctcgaagatctactgttccagcatgccgaacagcacggcttcggctggagcgtgcaccgctcgcacaccatggttggcattgccaacggcagcaacgcgatgaacatgggcgtgaccctggcggtgtatgcctcgctgtgcaaacacaccgggcaaccgttcgtgttccccggctcgcaggcacaatggaacagcctcagcgatgtgaccgatgccggcctgctcggtcggcaactggcgtgggccggcctgagcccggcagcgcgcaaccaggcgttcaataccgtcaatggcgatgtctttcgctggcgctggatgtggggcgagattgcgaaattcttcggcctggaagcggcgccctgcccgcagacgcccgaaccgctggaagcacgcctaagcgacaccgcaccggcgcaatgggcggagctggctgcgaagcacggcctggtggagccggacgtcaaccgtctcgcctagtggtggcataccgatgccgacctcggccgcgagatcgaatgcgtcaacgacatgaccaagacccgcgaattgggctttctggacttctaccacagccgtgccgcgttcctggaactgttc

SEQ ID NO: 52 - DNA, 1068 bp, Xanthomonas campestris
  CDS (121)..(948)
ctgtgcggcacggcaatcggtacagttcgcttcccggcattgccgttcccgcacagcgtttgcgggagccgcaccggccatcgcatccacgccactccgctgctttgccaggaacctttcatgccgtctttcaagtcgtttttgttacaccgcgtcagcccggtctgcctatgcgttgccactggttggggcacggtggcgatggcgcagaccgtgttgcccgacgccacgccagtgcacgcgcacgaaaccacccagcatgacgggcacgagtggggctacaccggcccggagggcgccgagcactgggccgaactggccaaggaaaatgcgctgtgcggcaatgggcagcagaacagtcccatcgatctgaaggacgcgatcgacgccacgctgggcaagctgcaactggactacggcgcagtgccgctggtggtgcgcaacaccggccactccatccagctggatctacacgccggcggcacgatgcgggtcggcggcaagcagtacgacgcactgcagctgcatttccatcatcccagcgagcacctgttgaatggccggcgcttcccgatggaggcgcacatcgtgcaccagggcccggacggaacgctcggcgtgctggcgatcttcttcgagaccggcaaggccaatccggccttccagcgcgtgctcgacgccatgccgagcgacaagaaccagacccggcaggtggccgatgcggtcgtgcgcgctagcgattttctgccgcccgccaatcaacgcagcttctaccgctacgaaggctcgctgactaccccgccctgcagcgaaacggtggattgggtggtcctgagtcagccggtgcaggtgtccagggaacagatcaacgcgttcgagcgcgtttacccgttcaacgcgcgcccgctgcagccgctggatcgtcgcttcctgctcaagagccactgagcctggcgcgagcgcgttgcctccatcgcagcgcgctgagcactgtgtttgctcctagctccgcgtctcctcgctaaaccgacagccgacatccgttgtgcgttggctgtcgggttggct

SEQ ID NO: 53 - DNA, 2576 bp, Xanthomonas campestris
  CDS (102)..(2456)
tttgatatgcagttgcagtccggcgcgtagagtgaacagccgaccgggcatgaccccgtgctggcggtcacccgcgacagctggcatggtgggccggtgcggtgatgacgcagatggccaacggggccgacccgcgcgccaccgacaacgacggcaacacgccgctgcatcacgcggtgcgcagttccgaccccggcgtggccgcgctgctgcgcgatgccgccgccgaactggatgcgctcaataacgaaggccactcgccgctggcgatggcctgccaggtcggcaactggcgcttggccaagttcctgctggaacgcggcgcgcagtccgagccggccggtggtgcgccggtgttgctggcggccgccgggaccgaggaagacgacccggctggcgtccaactgctgctcaagcacaaggcgcgcgtggatgcgcgcgaccgccagcgccgcagcgcgctgcacgaagccgcgctggccgggcacggcgacatcgtcgaggcgctgctggcggccggcgcaaatctggaagcgcgcgacctgctcggccgcacgccgtggctggatgccgcccgccaggcccgcagcgcggtgctggagcggctggtggcgcgcaagaccgacgtgctggcgatcgatggagacggccgcaatgcggtgatgctggcctgcgctgccgacaccgtgtcgccagcgctgatccgccgcctgctggagctggatgtccccgctgatgcgcaggacatgcacggccgccgcgccgtggatgtggccgccgaagcaggccgttgggcgatcgtgcgcctgctcgatccggcctatccgctgccctcggcagtcagcgacgcgcacggcgacggtgtcggtcccaccacgcaattaccggaccgcccgccgctggtgctgttgcgcgaaggcctgcagctgggccagcgcgatggcctggttgcactggcgcggctgtgcggcccggaagaactgggcggcctgctgcacgacgcgcacctggccctgaatgccgacgcggtcgactggctgctgcgccacggggccgatgccgaggtgcgcgatgcctgcggtgacgtgccgatgttcgcgctgctctcccgtggcgtcgaagcggtaccggcgctgcaggtgctgctgcgccacggcgtttcgccggccggcgccggcgggctgacccgcctgctcagcgcctgcgtgcagcacgacgcggcctcgcgcgggctggaacagttcgcgctggaattgctggaacgcggcgccgacccattcgcgccgtcggccgctggcgacccgccgctgtcgctggcggtgcgcctggggtggttgcgtctgcagcagtggctgctcgaacacggcgtggaccgcgaagcgcgcgacagccacggcatgaccgcgctgcatctggccaccgcgctgggccgcgaggcctcgctcaaattgctggtcaagcagggtgcctcgccggaagcgcgcgccgccgacgggcagaccccgctgggcgtggcgttgtcgatcggccggcgcgatctggccgattggctggactggcgtatctggtcgctgccacgccgcacgctgcgcgaggccgatgtgccggccgcggcgatgaccggcgataccgatgcggtgcgtcgcctgatcgacctcggcctgccggtcgatgcggtggacgcgcagggctgcaccgcactgctgcgtgcagccggcagcggccatctggccgtggtgacccatctgctcggccgtggcgcggacccgcagcgtgccgccaacagcggcgccacgccattgtcggcggcggtgagcatgcgccagaccgagatcgtggcggcgctgctggcggccggcgcgcagatcgaacaccgcctgcccggtggcgtcaccgtgctgatgctggcctgtgcgctgggcctgcctgacatcgtggcgcgcctgctcgccgctggcgccgacgtgcatgccaacgacgaccagggcctggcggcgctgcattgcgccgcactgtacggcttcaccgcgcgcgacaagatccgcctgctcgcgctgctggacaccttgctgctggccggcgccgacccggattacctggccggcgggcgggtcagcccgctgctgatgttgctgggcgcacgcgccgaacctggcaccgcctgcgacgagcaggtcgtactggccggtgtggagcgcctgctcgacgaagacgtggcgctggacgtggccgatcaacgcggcttcggcccgctgcatctggcagccctgcatgggctgcccttgctggtgcagcgcctgttgcgtgccggtgccgattcggaccgccgcgacgcgctcaaccgcaccccgcgcgagattgcgatcatgcgcggcttcatcgacgtggcgggccagttcgaaccggcgttgccgggggtgtcgtcgatggcgcgcttcctgcgcgaaggccgctgagccagcgcgccttcgcgccgctgcgggtgctgttcgtgtgcagccgcaaccggctgcgcagccccaccgccgagcacgtcttcgcacagtggccgaatgtgctcacgcaatcggccggac

SEQ ID NO: 54 - protein, 341 aa, Agrobacterium tumefaciens
MRLTLAPNGCLCESDTLRRDTGNNARSGMTATIRPADPEGVEGRAGKTMDILFDQALIEQNRRRAWTRRDDKALFLLDMAAEELADRLAIVERKFETAIELHGGTGVTARRLAETGKVDNIRRIETESAFATDGNIPETASMEHLPLAEASANLIVSPLALHLTNDTPGALIQIRRALKPDGLFLGAIPGSGTLHELRDVLLTAEAELTGGASPRVIPFADVRDVGALLQRAGFALPVTDAETYTVRYDSIFPLMRDLRAMGMANPLASRSRKPLNRAFFLRAAELYAERYSDPDGRIRATFSIIYVSGWAPHESQQKPLKPGSAKMRLADALKTTEVKLS

SEQ ID NO: 55 - protein, 147 aa, Agrobacterium tumefaciens
MANYWLYKSEPFKWSWEMQKAKGETGEEWTGVRNYQARNNMRAMKIGDKGFFYHSNEGLDVVGIVEVCALSHPDSTAEGDLKWDCVDIRAVCDMPQPVSLKDVKANPKLEKMSLVTSMRLSVQPVTEEEYLEVCRMGGLANPPKSPD

SEQ ID NO: 56 - protein, 232 aa, Agrobacterium tumefaciens
MKTLENGYRAIVIGASGGIGAALLARLKADPRCAFALPLSRSHDGLDITDETTVREAAARLEDQRGSFDLIFNATGALVVDGAGPEKSIRAIDADLMAKQFAVNAIGPALLLKYFTPLLQRDRRSVFASLSARVGSIGDNRLGGWISYRAAKAAQNQIIRTAAIEIARTNRQAIIVALHPGTVDTDLSQPFSKGRDRFTPDHSAVRLLETINGLDESQTGRFFAHDGSTIEW

SEQ ID NO: 57 - protein, 283 aa, Agrobacterium tumefaciens
MTEKSTPLFSFGIVADPQYADADPRPDMGRYYAESPKKLAEAINVFNSEDLAFVVTLGDIIDRGFENFDAILSVYDALRHPSVLLPGNHDFAVAPEHLSAIHARLGMKAPWHDFAMSGIRFVVLDGNDVSLFAPPPDDPRRLVAQERLKRLWDVDAINAQDWNASLSDKQFEWLRTILQQADAAGERVIVLCHYPVYPENAHNMWDAPRILDLLGSHSSVAAWFNGHNHEGNYGTLGTTHFVNFKGMVDTPDQNTFAIADVFADRIAIRGFGREEDRVLQYLR

SEQ ID NO: 58 - protein, 321 aa, Agrobacterium tumefaciens
MVGWWPVGRRQAWHRFFPCHRRPALRCDAHSFFAPGKHIMAHRILTLGEITDGFDVVLSDVWGVLHNGVSAFPDAAVALHEARKAGKTVVLITNSPRPAPGVIAQLRVLGVPDEAYDRIITSGDVTRGLIAEGPKKVFLLGPERDMPLLEGLDVERVGEAEAQSVVCTGFFDDETETPEDYTEMLKGFIARKVPMICANPDLVVERGERIIPCAGAMAAYYEQLGGEVRIAGKPHAPIYEACLAAAKDVRGAFAKDRVLAIGDGMPTDVKGAIASGLNLLYISGGIHAAEYTLNGQTDEALLNAYLKGQGAAPGWWMPRLA

SEQ ID NO: 59 - protein, 363 aa, Agrobacterium tumefaciens
MGIRPSADEQDQCFMTRITIVTDAWHPQVNGVVRSIENTNTELARLGVDVRMVTPQSFYSIPCPTYPEIRLSVAGYRRVAVEIEKSQPSFVHIATEGPLGFMARRWCVKNHMRFSTSYHTRFPEYVAARFPVPESWLYAFVRWFHNGGNTCMVATPSLETELEARGVRNLKRWSRGIDAELFHPRTKTKLPFDLPRPIFMTVGRVAVEKNLPEFLDLDLPGSKIVIGDGPARYELQEKYPDVLFTGIKTGEELADAYAQADVFVFPSKTDTFGNTILEALASGVPVAAFPVTGPIDILGGNPAAGALDNNLRDACLAALHCSPQAALALSKSYSWEKASRQFLDNVIHAAGKSLPLLSRSRLA

SEQ ID NO: 60 - protein, 185 aa, Agrobacterium tumefaciens
MLLRWSLIVLLTFGLSLDSAAATETDAVGAFHHAVQVGDVQTIRTMIAADPGLATSRDKFGFQPIHLLDMYPEEEVLNLLLKNGADINAANDEGVTILHIITDPDTVSLLVKRGANIEARDERGWTPLIMQANNQQNGPDVVAALLAQGADPNAEGFAGETALSFAKQTGDAALVRMLIASGAKN

SEQ ID NO: 61 - protein, 281 aa, Agrobacterium tumefaciens
MEPTENLDVCLATLRDTDRDRYLACLLSPADKRNALAALYAFNAEIARIRDSVREALPGEVRMQWWRDLVDGNPHGDSQSHPVAAALLTTIEHYRLPRPVLGNMIEARIFDLYDDLFEDRNALEGYAGETASALIQLASLVLSAEDAAHSAEAAGHAGVAQAMAGMLLLMPLHRRRGQVYIPADMLAAAGLDRETFLAGQDRQRIGIAVEIFAAHALDHIEKARRATISKNLFAAYLPVALSGPVIAAARKAGAGVFDGDLQLSQLRRQWLLAKASFLKRF

SEQ ID NO: 62 - protein, 300 aa, Agrobacterium tumefaciens
MPLRCFCNVERRFCRNALAKVMPIQSSIALAVSCAQGRSQARFSDQSSSFRFSSPFETGIKQPMDTKPKNLPDAADFAHVSEWVFDLDNTLYPHHVNLFSQIDRNMTAYVAELLKLDPEEARVLQKRYYHDHGTTLQGLMINYGISPDEFLERAHAIDYSALKPHPELGEAIKALPGRKFILTNGSVKHAQAAAGALGILDHFEDIFDIVAANYLPKPASATYEKFAALAKLDTKKAAMFEDLPRNLAAPKALGMKTVLLVPSNLEGVIMERWEIPDVTDEHIDYITDDLTGFLAKTIAR

SEQ ID NO: 63 - protein, 290 aa, Agrobacterium tumefaciens
MHVMIFGAGYSGKAIANALKTKAASVCGTTRSADKFANLAAAGMTPLLFDGVHLDDDLIAAMRNVTHLVQSIAPGKDGDPLFALLGGDLKKLLPNLKWLAYLSTVGVYGDHHGAWVNETTPCRPVSARSVERVAAEAAWTEAAQSADVPLSILRLSGIYGPGRNTFMNFEKGTARRLVKKDQVFNRIRVEDIGAAIAFLALKNERGIFNVTDDEPCPPQDVVSFAADLMGVVPPAEQDFGTADLTPMARSFYGENKRVSNAKIRELGFDFRFPEYRQSLKQLWESSLWRG

SEQ ID NO: 64 - protein, 315 aa, Agrobacterium tumefaciens
MTPDFLVTHSGGFHADELLSSVVLTRLFPQARLVRSRAPEWITPDADRIIYDVGGAYDAEKCIFDHHQRGAPLREDGQPYSSFGLIWKHFGRDYLATSGIPEDHVETLHASFDAGFVLPVDLVDNGALSPSIAGPLATLTLPVLLETLKPVFDDTDPEADDRGFIAALAVARSFVEAKLATGAAKLRAEALVQKAIADTGEGHILELPMGMPFRSAIVKAGADHLLFVVHPRDNDWCLTGIRRADEGFELRADLPAAWAGLTGKELEAVCGVEGASFCHNGRFIAAAKTREAILTMAELAVKEALASAQTTAAKQ

SEQ ID NO: 65 - protein, 599 aa, Agrobacterium tumefaciens
MQGDKPVPHRPGRSDGSTGPMLDKGGGRGKLSSPERFREWTRKTMAKQDELVFVPLGGVGEIGMNLALYGYGPESKRQWIMVDCGVTFAGPDLPGVDLVLPDISYIAEQKKNLKGIIITHAHEDHYGAMNDLWPGLNVPVYASPFTAGMLEAKRAYEGTRAEIPVTIFKAGDRINVGPFEIEAVGVNHSIPEPMSLIIRTPLGNVIHTGDWKIDEAPSLGPLTDEARFRAIGEEGVLALMCDSTNALRDGVSPSEHEVSEGLRQIIESAEGRVAVTTFSSNVGRIRSIAQAAEAAGREVLLLGSSLKRVVNVSQDLGIMEGIKPFLAEDEYGYIPRDRVVVILTGSQGEPRAALAKLSRDEMRNVALASGDTVVFSSRAIPGNEKAIIEIKNGLIEQGVHIVTDNEALVHVSGHPRRNELLRMYEWTKPQILVPVHGEAAHLVAQKELAEQAGISQVPKVRNGNVLRLAPGPAEVINDVPHGRVFKDGNLVGDMDEMGISNRRKLSFAGHVAVNVVLDSRYDFLGDPDVVPFGLPEFDDEDEAMEDTLYDAVLGAVESIPRARRKDLALLRESVRRAVRAAANQSWGKKPIVTVFVTKV

SEQ ID NO: 66 - protein, 791 aa, Xanthomonas campestris
MTHAHPNDDVNDAALSLSRRRFLAWSSLAVAAGFLRFPAQAAAAQPGSIRAVPLAQVRLTPSLFLDALHTNRRYLMRLQPDRLLHNFVLYAGLDPKAPAYGGWEADTIAGHTLGHYLSALALMHAQTDDAQCRTRASYLVAELARCQAHAGDGYVAGFTRKNAAGQIESGRAVFDELKRGKIEPLPFYLNGSWAPLYTWHKLFAGLLDVHVRCDNAQALQVAVALAGYLQGIFAALDDTQLQKVLSCEFGGLNESFVELHVRTGDAQWLALAQRLHHHAVLDPLIAQRDELVHQHSNTNIPKLIGLAREYEVTGDAASGAAARFFWNTVTDHHSYVIGGNGDREYFQQPDSISKFLTEQTCEHCSSYNMLKLTRHLYQWGPQAAYFDYYERTLLNHVMAQQHPRTGMFTYMTPMLAGEARGWSSPFDDFWCCVGSGMEAHAQFGDSIYWEDGQGVAINLYVPSRVRNAAGLDMTLHSALPGQGSVSLRIDAAPAAQRTLSLRVPGWAAAPVLQLNGAVVDAAAVDGYLRVTRIWHPGDTLNLSLQMPLRLEATPDDPAWVSVLRGPLVLAADLGDAATPWSGKTPALIGGDEVLQQLQPAAGQGSYVYSDGAQQWRFSPFYAQFDRRSAVYLEHRSANDWQQRQAEQDAAAARQRALDASALDWVALGDDASEKAHALQSDGSYALSYRRRAGRDARTGGFIAFTLRNTATARVLRLRYWGDETRRRFRILADGELIANERLDGNRGLDFVDVDYALPAAVAGRAILQIRIEPETGYSAGPAFGCWVLESA

SEQ ID NO: 67 - protein, 293 aa, Xanthomonas campestris
MHCSGMTNTSIPADLGDWIIAQAIAGHPPEHTLQPLLEAGWSEQDAIDAVEALVRAHVQAHAQAHGLPVPVPVRVPAPLQADASSLLDLGDRQVQVLVSLMLPRVVVLGGLLADDECDALIALARPQLARSRTVDNRDGSEIVHAARTSHSMALQPGQDALCQRIEARIAQLLEWPVEHGEGLQVLRYATGAQYAPHYDYFEPDAPGTPVLLQHGGQRVASLVMYLNTPERGGATRVPDVHLDVAAVKGNAVFFSYDRPHPMTRTLHAGAPVLAGEKWVATKWLRERPLHAPT

SEQ ID NO: 68 - protein, 296 aa, Xanthomonas campestris
MTTLIAPRVHDIGGLQVRRAVPTLQARSVGPFVFVDHMGPAVLEPDHGIDVRPHPHIGLATVTFLWSGEIGHRDTLGSDQVIRPGDVNWMTAGRGIAHSERTPGPERAREHAMHGMQTWIALPRSAEETAPAFHHHAATSLPQQRRNGVWLRVIAGRAYGEESPVQVFSGTLNVALDLDADAEIDLDTGHAERALYILEGEAQLDGADVPARHLIVPSAGTRGRLRAKTPLKAMLLGGEPLDGPRHLWWNFVSSSQERIEQAKDDWQAGRFGTIPGDDQEFIPLPETPAPKPVNYP

SEQ ID NO: 69 - protein, 378 aa, Xanthomonas campestris
MRYAIVTETYPPEVNGVALTVHGLETGLRARGHQVDVVRPRQAADSTPADTVLVRGASLPRYPGLKFGLPATRRLTRHWATTQPDAIYVATEGPLGWSAMRAARRLGIPVATGFHTRFDEYLPDYGAAWLQGTALRWMRRFHNQAEATLVPTRELQQFLRGSGFERVQLLARAVDSLQFDPSRRDAALRAEWGIEGEGFAAIYVGRIANEKNLPLAIRAFRKLQQLRPKARFVWVGDGPAREKIAHENPDFIFCGIQRGDALARHFASGDLFLFPSRSETFGNVTLEAMASGVATVAFDYGAAREYLRNGQTGAAVEDDAAFVQAALTLTENDDVRQRMGHAAAQAMKKLHPDNVVSDFEALLLGITAARGRYVVDAA

SEQ ID NO: 70 - protein, 491 aa, Xanthomonas campestris
MVWGGIAHERLQLNEDTLYAGGPYDATNPQALAALPQVRALIFAGRYAEAEQLADAKLLSRPLKQMPYQPLGDLLLDFDRADGISEYRRQLDLDTAVATTTFRSGGAVHRREVFVSAQSQCIVVRLSCDRPGGISLRVGIDSPQSGEVTVEQGGLLFSGHNGSFAGIDGKLRFALRVMPQVKGGSVTAVRDRLRIQGADEVVLLLAAATSYQRFDAVEGDPLALTAASLQKAGKLSYAALLRAHLADHQRLFRRVVIDLGSSEAATLPTDERVQRFAEGNDPALAALYHQYGRYLLICSSRPGTQPANLQGIWNDLMQPAVGEQVHHQHQHRDELLAQRSQRTARVRGTAGSHAVRSRPHRCTHRPCDVRRARLGGPQQHRSLAPGRADRRCAVEPVADGRGVVAAAVVGPLGLRPRPCVSGQDLSVVQGGGRVLRRHPGARPRHRRDGDQSIDVAGKPASVRGGGLRRPDHGCAAIARPVRAVHRDEQVA

SEQ ID NO: 71 - protein, 312 aa, Xanthomonas campestris
MSARIPGLDITRFDSDREAFVAELGAAYRQWGFAGIRNHGIAQSDIDAAYEVFKAFFALPDEVKRSYHVPGSGGARGYTAFGVETAKDSKHFDLKEFWHIGREIPDDSPYRAVMAPNLWPTEVPGFRERGYHLYQQLDQLGSRVLSALALHIGLPQDYFADKTDHGNSILRPIHYPPITSDDIPNVRAGAHGDINFITLLVGASAAGLEVRSNEGEWVPFTADADTIVVNIGDMLQRLTNHVYPSTIHRVVNPPGEEARKPRYSVPFFLHPNPDFLIDVLPSCISADNPSRYPEPITAHGFLEERLREIKLK

SEQ ID NO: 72 - protein, 344 aa, Xanthomonas campestris
MTDSVRFPAEWESQSAILIAWPHAGTDWAERLAEVEDTYIALVTAITRFQPAIICVADDDLQIYAEARLRSARVEMAQVRFVVAAYNDTWLRDSGPITLRHGDGFRLMDFRFTGWGGKFEASLDDQLVSSLDAASVFVPAEVQTVDFALEGGAIETDGAGTLLTTWKCLHERHPQRSRDSLSSDLATWLAQDRVLWLDHGYLEGDDTDAHIDTLARFAGPDAIVYQGCDVPSDSHYAELQAMGEELAALRKADGQPYRLFVLPWAEPILDQGRRLAASYANFLIVNGAVLMPAYGDAADAAAQAVLADAFPQHQIVPVPCRPLIWQNGSLHCLTMQLPAGLLAA

SEQ ID NO: 73 - protein, 648 aa, Xanthomonas campestris
MSTPTDSHDAGRRQLLQGAAAGLALGIAPAALTANRTPPMVDLILRNARITTLDPRQPSATTLAVADGRIVAVGDDAQIMAMAKQARVIDAQQRRVVPGLNDSHTHLIRGGLNYNLELRWDGVRSLADAMALLKAQVDRTPAPQWVRVVGGFTAHQFVEKRLPTLDEINAIAPDTPVFLLHLYDRALLNRAALRACGYDKTTPDPPGGRIERDKLGNPTGLLLAKPNALILYASLAKGPRLPIEYQANSTRHFMRELNRLGITSVIDAGGGFQNYPEDYQVIEQLHAAGQLSVRIAYNLFTQNKGKEVADFRGWTEVLPVRKGDDLLRHNGAGEMLVFSAADFEQFSEPRPELAPEMEPELEQVVRLLVEKRWPFRIHATYDESISRILDVYERVNADMPFDGLHWFIDHAETIAPHNIDRIRALNGGIAVQHRMAYQGEAFVQRYGVQAARHTPPVRRMLAAGVPVGAGTDATRVASYNPWVALSWLVTGRTVGGLALYGDENLLEREQALRLWTQGSAWFSDDEDRKGTLAPGQLADFIVLSDDYFRVDDAAIADITSVLTVLGGAVVHGDGDFAALAPSLPPAMPEWSPVNRYGGYHVGGLASVAHAHGPGCRTHGAHGHAAHHAAPSDDLRGFWGALGCACFAF

SEQ ID NO: 74 - protein, 1168 aa, Xanthomonas campestris
MGLSTIKLSGSKSFGRTRPRCTCPTNMTGIVGPNGCGKSNIIDPVRWVMAQSSASRLRGDSLTDVIFSGSSARKPVSQATVELIFDNSDHTISGEFASFNEISVKRLVSRDGTSAYYLNGTKCRRRDITDLFLGTGLGPRSYSIIEQGMISQIIEARPEDLRVYLEEAAGISKYKERRKETETRIRHTRENLDRLGDLREEITKQLAHLQRQARQAEQYQALQEERRIKDAEWKALEYRGLDGRLQGLREKLNQEETRLQQLIAEQRDAEARIETGRVRREEAAEAVAKAQADVYQVGGALARIEQQIQHQRELSHRLHKARDEAQSQLQELTQHISGDSAKLAVLREAVADAEPQLEQLREDHEFRQDALREAEARLADWQQRWETHNRDTGEASRAGEVERTRVDYLDRQSLEADRRREALVNERAGLDLDALAEAFEQIELRHETQKASLDGLTEQVEARKHALGGLQEQQRASQGELAEVRKQAQAARGRLSSLETLQQAALGQEQGAAVAWLKARGLDSAVRVGERISVESGWENAVEGALGQLIEGVLVEAPEQLVDALGELGDGRIALVSSATDNVQFAPTSLAAKVQGPIAIRRLLARLHAAEDLDAARSLQCNLPDGDSVITRSGERLGEGWVRVSRSGAAKQGALLREREIQELRSQIDTLQEREGDLEEQLASFREQLLAAEQQREDAQRQLYMAHRSVSELAGQLQSQQGKVDAARGRIERIEAELGQLLETLDTSREQAREARAKLEDAVVRMGDLQGTREALESERRQLTDARDQARDAARGVRDAMHALALTLESQRTQITSLSQTLERMDSQRGQLDTRLEDLVAQLSEGDSPVETLEHEHQAALSERVRTERVLGEARTMLDSIDGELRSFEQTRQQRDEQALAQRERISQRKLDQQALVLNAEQLAAAVVKAGFVLEDVVNGLEEAANPAEWDAAVGQIDARMRRLEPVNLAAIQEYGEAAQRSEYLDAQNVDLTTALETLEEAIRKIDRETRGRFKDTFDRVNSGVQALYPRLFGGGHAYLELTGEDLLDTGVTIMARPPGKRVSSISLLSGGEKAMTAVALVFAIFQLNPAPFCLLDEVDAPLDEANVGRLANMVKEMSEKVQFLFVSHNKATMEAARQLSGVTMREPGVSRLVSVDLEEAARLAGAA

SEQ ID NO: 75 - protein, 155 aa, Xanthomonas campestris
MSRKVAARKSRIHGNGVFAVAALSKGERIIQYKGRLRTHAEVDADDTGDVESGHTFLFTLSDDYVLDANYEGNIARWINHSCNPNCEAVIEEAEGDDRSKDKVFIEAKRAIKPGQELTYNYGITLGERHTPRLKKIWECRCGSKNCTGTMLQPKR

SEQ ID NO: 76 - protein, 355 aa, Xanthomonas campestris
MPLSRLLTCLVASLAAYAVQAKSPMPPPSPIMLSEFIADQAPTPQAHASTLLETRDGTLLAAWFGGAHEGAADVGIWLAQRGPQHWQTPRRIADGAHAGVDGAALPAWNPVLFQSATGPVQLYYKLGPNPRDWWGLRITSSDNGAHWSPPQRLPDGILGPIKNKPVQLPSGRILAPSSGEDRGWRAHLEWSDDDGAHWQRGLPLNDAATIGAIQPSLLLHADGRLQALGRSQQNKLFSAFSDDGGLHWQPMQLLDVENPNSGTDAVMLHDGRALLVYNPGIAGKQWWDGRGTLAVAVSDDGVQWRRVLTLEDSPKDEFSYPAVIQTRDGLVHISYTWKRQRIKHVVIDPAKLKVR

SEQ ID NO: 77 - protein, 605 aa, Xanthomonas campestris
MASTLRPIRSLMAVAIALAASPAMADSAFDQTVFFGDSLTDSGYYNPLLPAASRAVTGKFTTNPGWVWAEYVGDHFGTNAAPNGNGQTGDNYAAGGARIQASSVSELGAAPSVTSQVNTYLAANGGQANPNALYTVWGGANDLLAAATAPAQAQAIIGSAVTAQVGAVGALQAAGARYVMVPTIPDVGITPRFRAGGAAAMAQGTAAATAYNTALFNGLQSAGLRVIPVDTFHILQEVVADPGTYGFSNVTGTACNPALALPACNPTSLVAANAPNTYVFADGIHPTTSTHQILGQYAISLLEAPRLQQVLTRSAQAGGRARADQVAWHLDGKPEADGLRWWGSVRGDIQRYDDADLYDGMAPAGLFGVDWTAGDLVFGGFAGFGGMDADFGNRNGSFKQDDTTLGGFVGWYTGPVWVNAQVSYSWLSYDVDREVQLGPATRVHSGSPDGSNLTAAVNAGYSLGEGNVKYGPVVGLTWQKLKLDGYTESNASSTALGYADQDIDSLVGRIGFQVRLDGAPVKPYLQATYDHEFKDGTEASAWLQSMPEVGMYTVPGQNFDRNYATVVLGARTGIWGLQSNIGLSTTTAQRSARDATVFVNFSGNF

SEQ ID NO: 78 - protein, 202 aa, Xanthomonas campestris
VLFADYTHNLHAGNPNEVFAFDLSDHTVITGLLIGGLIPYLFGAMAMEAVGRAAGAVVEEVRRQFRDIPGIMAGTAKPQYDRAVDMLTRSAIREMIVPSLLPVVVPVIVGLLLGPRALGGLLIGTIVTGLFVAISMTTGGGAWDNAKKYIEDGHFGGKGSEAHKAAITGDTVGDPYKDTAGPAINPLIKIINIVALLLVPLL

SEQ ID NO: 79 - protein, 565 aa, Xanthomonas campestris
MPSLNAAPKRGAGRHAVRGAYSAAIRPHEHRDAQQALSGRRVRLRRSTRRAPPFVRPDRTTSILLGGVMLEHYGLMLALGCAVLAIVYGMLSARWVVAQPSGNARMQEIAAAIQEGARAYLNRQYLAISIAGVVLFVLVGLFLSWYTAIGFAIGAVLSGLAGYIGMNVSVRANVRTAEAARHGISKAMDVAFRGGAITGMLVVGLGLLGVAGYFAILQAIGLPLEQNLHALVGLAFGSSLISIFARLGGGIFTKGADVGADLVGKVEAGIPEDDPRNPAVIADNVGDNVGDCAGMAADLFETYAVTVIATMLLGSLTLAETGPHAVLYPLVLGGVSIVASIIGAAFVKVKPGGSIMGALYKGVIVSGVLAAIAYWPITQALMRDNAHGAMSLYACALIGLVLTGLIVWITEYYTGTQYGPVQHVAAASTTGHGTNIIAGLGISMKSTALPVIAVCAAIWGAFHFGGLYGIAIAATAMLSMAGMIVALDAYGPITDNAGGIAEMAELPPEVRNVTDPLDAVGNTTKAVTKGYAIGSAALGRAGAVCRLHPQPARRQSERGIRVRSV

SEQ ID NO: 80 - protein, 238 aa, Xanthomonas campestris
MPPRASHSATYPRAHPQGQRTQGGDAVCRQRKELHMRGVVSQLKRLGIRGIVLGMACAIAGCGQWASSFSPTGTDLSSMGRGVDFYLKDTEGTPRPLRSFRGKVLVLFFGFTNCPDVCPTTLARAAQVKRALGADAAQVQFAFATLDPQRDVPNVLRTYVKAFDPDFIGLTGSEGEIRVAADNLKVVYAKVPMGKTYTIDHSTRSFVFDREGKLRVGLTHEQTVETVAADIRQVLALQ

SEQ ID NO: 81 - protein, 406 aa, Xanthomonas campestris
MHDVDCIVVGAGVVGLAIARQLAMQGNEVLILEAASAIGTGTSARNSEVIHAGLYYPPGSLKAQLCVRGNALLYAYCRQRNVPHQRCGKLIIACDQAQTAQLEALQRNATDSAAPGVVPLSSAQVQARAPALRCAAALESTTTGIIDSHALMLALQGDAERHGATLALRAPVQSIEPLVAGFRVCTAGGEAMGLTCRWLINAAGHGAPALAAGTEGLPATARVRAQYAKGSYFTLAGRSPFHQLVYPLPEPGGLGVHLTLDLQGRARFGPDVEWVAQPDYHCEPARAEHFYAAIRRYWPALPDDALQPGYCGVRPKISGPGEPAADFRIDGPALHGMAGLVNLFGIESPGLTACLSIAEHVAALLGDEARATAMRVLPVKVVLRRGAPHVPDPGAAAARRQQWRAF

SEQ ID NO: 82 - protein, 228 aa, Xanthomonas campestris
MLTPDLGPASCVPAFVSAFTHSQFPTPDSHPMKSICVYCGSNAGNKPAYAERAIALGDRIAKQGLRLVYGGGNVGLMGTVANAVLAAGGEVTGVIPQQLADWEVAHRGLTTLEIVGSMHERKMRMFELSDAFVALPGGFGTMEEIFEMLTWRQLGIGNKPCAFLDIEGFYAPLIGMIDRMVEERFLHPDQRSDLWYGADIEQMLEWMQHYTPAQASKWIDEKRRSTLV

SEQ ID NO: 83 - protein, 199 aa, Xanthomonas campestris
MNIRVALPQAQVHWCRGWLQAAHADALMQALLDQVQWEVHRIRMFGRVVDSPRLSSWIGDADASYRYSGTQFAPQPWLEALQPVRTRLQDETGSPFNSVLVNRYRSGADAMGWHSDDEPELGAQPVIASLSLGAARRFAFKHRHDAALKQTLELGHGDLLLMGGDTQRHYKHALPRTVKPVGERINLTFRQIAVRVSQR

SEQ ID NO: 84 - protein, 156 aa, Xanthomonas campestris
MTARKRYWLMKSEPDTFSIDDLERVGTEPWNGVRNYQARNFMRDGMQVGDGVFFYHSNCKVPGIVGIAKVASTAYPDETQFDRTSDYHDPKAKREDPRWMLVDVAFERKLKRTIALDEIKQHADALGEGFPLIARGNRLSILPVTAAQWKLLLAME

SEQ ID NO: 85 - protein, 334 aa, Xanthomonas campestris
MSIEQLAETAQAMVAPGKGIIAIDESTSTIAKRFSGVGIENTEENRRAYRELLLTTPKLSDYISGAILFDETIRQKTKDGVPFAKYMADHGIIPGIKVDKGAQPLAGMPGELVTEGLDGLRARLEEYYTLGARFAKWRAVINIGEDIPSGTCIEANAHALARYAALCQEQGLVPMVEPEVIMDGDHDIETCYEVTEATLRSLFGALYEQNVVLEGTILKASMVISGKGCEEQASVEEVAESTVMCLKSTVPAILPGIVFLSGGQTDEQSTAHLNEMHQLGNLPWPLSFSYGRAMQQAALKLWSKDMTGNYAKAQQIIYERAKENGLAALGKWKG

SEQ ID NO: 86 - protein, 1154 aa, Xanthomonas campestris
MLMVRKAFRRGALWLLCGCVGWTAVEAAPAKVDPPGPYDVRVLAGGVGLSKTLAADAPMLAADADWSVSGWVRPAMDVPGTVRIAGLGDARGDGRFFVIDNGRLGFAQGDSVLRSKQALPVATWTQIAAVAQGARLTLYANGRQVASGTRVSRAVAPALVFGPRVPADAASLHFGGDIANFTARAGALDAAAIAQQARHAPNEILQRFEDASPGWRVQTKQMAGQLEPQPAATLPRSAAPFSAPVAKPVTNAPALQPLDALSWRVGGWQLAAAPDLGTRGGAELSRGAAPSSSAWHVATVPGTVLTTLVDRGVYPDPDIGLNNMAIPESLSRQDWWYRSTFELPASTQGKRLELVFNGINYAGEIWVNGVQVGRTRGAFARGRFDVSSYLTPGRNAIAVRVSPPPHPGIAHEQSMTAGVGENGGMQALDGPTFIASEGWDWIPAVRDRNAGLWQDVQLHATGPLAVGDTHVVTAKLAPGHRRAELEIAVPLRNTSGTAVQGTVQLAFGDVRIQREVNVPAGGTTLTLTAADTPELVVTNPRLWWPNGYGEPSLYTLQVGVAAGGVQSDLQQLRFGIREVTYELSLFDERGALRRVLVDLNQARQRGERIVDVRHAAIRPVPGGNAQSLYPGALASPAVTQLDDSTLAPHLVLRINGVRIAIKGGNWGMDDWRKRVSRERLEPYFRLQRDAHFNVVRNWVGQNTGASFFDLADEYGMLVLNDFWQSTQNYNMEPADAALFLANAAEVITRFRNHTSIVLWFGRNEGVPAPILNEGLDALVAALDGTRWYTASSNEVNLQGSGPYNYREPVAYFNKLAQGFSVEVGTPSFSTLESFKASVPAVQDQWPISDSWAYHDWHQSGNGDTASFMRSLSDKLGAPTSLDDFERKAQLLNYETYRAIFEGFNAQLWTKNSGRLLWMSHPAWPSNMWQIYSHDYDTHAAYYGVRNAAEPLHVQMNLPGHEVVVVNNTTTAVRGLRVRAETYAADGRLLQQREQAVDAAAVAVSAPVLQLTPLLANAKDLGFVRLQLLERDGTVRSRNFYWVARDAAAMRCLDALAPVPLELTTQLQAGDAPVLSATVRNPSQHVALGAKLTLVDAQGQRILPAYYSDNYLNLAPGESRTVEIRGPAEASLRAARVQLRGWNLAPSSGTASGSP

SEQ ID NO: 87 - protein, 335 aa, Xanthomonas campestris
  unsure (1)..(335): unsure at all Xaa locations
MEREGCLRPDWGWQALHLTLWDGDALVAAAPGYLKNNSHGEFVFDHAWAHAYARYGQDYFPKWLCGVPYSPVTGPRLLGDPRWHTHLLDGMRTLVESAGLSSAHVNFYPANEDAAFTAEWLERNDLQYHWHNATGWADFEAYLAAMNHKHRKNIRQERAKVQRAGVRFRVLHGDEASADELQAMYGFYLKTFEEYGNSPALTPXFLQHLAQHLPRQLVMFLAELDGQPIAGALCLRGGDTLYGRYWGGEALPGLHFETCYYQGIEYCLCEGLTRFEPGAQGQHKIARGFLPTLVRSRHWIADPGFREPLAQWCADERAAVAQHAVELAEHSPFRD

SEQ ID NO: 88 - protein, 251 aa, Xanthomonas campestris
MRPSLLLLPLAFCAACSRIPPPVTPPGAEETTKTNVLEAGAKVLQSNGPVGKLDIYLVGFHPMKDAPEQQMEAHHYCQQVNQDLAQCALYDGNTAEANLTGIEYIISESLFAQLPAQERAFWHPHNGEILSGQLSAPNLPLVAEKELMRSKMNSYGKTWHTWHSRKGTAPGDTLPLGEPMLAWSFSRDGEIQQPLIEQRDKAVTISTAERRADRKDLTELAKPQHGVDALHRHFPEATPIPGVVDAEATAQ

SEQ ID NO: 89 - protein, 635 aa, Xanthomonas campestris
MTVTCEMDALGRAPDIGRERGLLGRSLVSAGVITDEQLRAALALQQRWNSRLGDVILAQRGVPAQRFYAIVAAHFGLQFVDLVQQPPDPELLTATDLDVYAQRLILPWRREDGVLVLAVADPDPALFAWAREHYGAQVRFVGTAKFDIIWSLQRYADEQLTDNALNLLAAHAPTYSARQVVTRGQKFTLWAIAAVLVAAIAAFPIITLITINVLVALGFLATFGLKLLLVWFGSRHRIDIKVTEDEVAALRDDDLPVYTVLVPMYKEPEVLPILANALRKLDYPISKLDVKLVLEADDFDTIEAAKKLGLEAFFEIIRVPPSQPKTKPKACNYALHFARGELLTIYDAEDKPEPDQLKRVVAAFRKAEKDVVCIQARLNYYNADENWLTRMFTLEYTLWFDFYLPALEYLRIPIPLGGTSNHFRLDVLRQVRAWDPYNVTEDADLGVRLIQNGYRVNVVNSTTFEEANVSIPNWIRQRSRWLKGYMQTWLVHMRDPVHLYRSTGFKGFWGFQFFIGGNFFIALGVPVMWTLCLISMLSGARIFDATFPPWLATISLVNLLLANAFFVYVTLVAAFKRDYFKLAPYALTVPFYWLLQSIAAYKGLWQLIRNPFYWEKTTHGISKHSEQERRAALEE

SEQ ID NO: 90 - protein, 298 aa, Xanthomonas campestris
VDSQTDEPLDEARADRIDWLRALPFIALHLACFAVFWVGVSWFAVGMAVALYALRMFALTGFYHRYFSHRAFKTSRVVQFVFAAIAATCVQRGPLWWAAHHRNHHRHTDTGRDPHSPAQHGFWWSHTGWFLTPRNFRTDWEVIPDLRRFTELRFLDRFDIVMPVLLALGLYLLGDWLAAAAPGLQTNGAQLLVWGFVLSTVALFHATFTINSLAHRFGSRRFDTRDDSRNNWLLALLTFGEGWHNNHHFFPGSARQGVRWWEYDMTWYGLTVMSWVGLVWDLKPMPAMLTRRAGRVAR

SEQ ID NO: 91 - protein, 416 aa, Xanthomonas campestris
MSGGRIAVVGSGIAGLGAAWLLSRRYEVTLFEAADYLGGHTHTHDIQLEGQRYAVDSGFIVFNPQHYPLLTRLFAEIDVASQSTTMSFSVHDARSGLEYNAGSLGGLFCQRRNLLSPRFWGMLADLRRFYRQAPAVLTSDERFSTLGEYLHRHGYSDTFRDQHLVPMASALWSSPSQRILEFPMGQLIGFMANHHMLQISGRPQWQVVRGGSSSYVRALRRRWQVQERLSTPVHAVRRAGNGVVVTSLRGDEHFDEVVLACHADDALALLSDATPAEQQILGAITYQNNDTVLHTDASVLPRDRRAWAAWNAHVPADPQAPCTVSYWMNALQSINAPQPFIVSLNRDQDIAPEKVLRRMRYRHPVQNAAALDAQARKAQIQGQRHTWFAGAAWGFGFHEDGLRSGVDVAHALGVPW

SEQ ID NO: 92 - protein, 260 aa, Xanthomonas campestris
MTVWPLLHVGVFAAVVMVLGWLWQRRSGNAGPVDVLWAGCLAVAAPYCAWMSDGAPLPRVLVAVLGGVWGARLAWHLGVRVFGDPHEDGRYRALREHWNGDQRKFLGFFLAQAVVVVLFSVPFLAAASNPNPAWSVWSSLAIVVWLIAVGGEALADRQLSAHKANPANRGKTCRTGLWRYSRHPNYFFEFVHWFAYLALAVGAGPWPVALCALGPVVMFVFLYRFTGIPYTEQQALRSRGEDYAQYQRSTSAFFPLPPSS

SEQ ID NO: 93 - protein, 263 aa, Xanthomonas campestris
MPATSEKAMRLLNKTIVVTGVASGIGAEVARLARFEGATVIGIDRTPVHMSLHGFHQADLGDPAAIDALVQALPERIDALANVAGVPGTAPLEAVARVNYLGLRHLSHALLPRISRGGAIINVASILGAEWPQRLDLHKQLAAAASFADGSAWLAAHPVAHDTCYQYFKEALIVWTLLQSWPWFADHGVRVNNVSPGPVFTPILGDFAAMLGAERVQEDAKRMQRPAYADEVAEAIVFLASDAARWINGITLPVDGGLASTTL

SEQ ID NO: 94 - protein, 270 aa, Xanthomonas campestris
MKTFSPLLLTLSVCAAALQPTAALAWGPQGHRLVARVAETELTPQARAQVSQLLAGEADPSLAGVATWADELRANDPDLGKRSGPWHYVNLGEHDCGYVPPRDCPDGNCVIAALEQQTAVLADRNQPLAARRQALKFVVHFVGDIHQPMHAGYAHDKGGNDFQLQVAGKGSNLHALWDSGMLNDRHLSDDAYLKVLLALPATAPTSPALPPPGAAWAQASCRIAITPGVYPDTHVLPPTYIATYRPIAETQLRVAGDRLAALLNAALRSP

SEQ ID NO: 95 - protein, 919 aa, Xanthomonas campestris
MCPRSAPPHVLTIAKDAFVMSLPLHRLLLCASCLGLLLLAGCGKPQDGRDGQEAAKPAASATPAAQNDPAAEDARVLIAQLREQLDRHRRIIVLLADEAAQSPRDRATSSSVGQQLFHENLEQRERIAARFETVLASKASTRFATLATVLDDVESAPDLYDADRLAFREVLRDLHARIGRDSALPAVKLHQRIGEDLEALDEIERTYNTELTQIFSRFDRSRAITPKREKWDDYVAHLRTLYTREKILRDHGVVEPYPFSARDSESEIFGRDLPPKTVVLTFDDGPHKAYTEEIVAILKRYDVPGVFFEVGRNLGNLDAQGKPTLAPLAKISHSLMEEGYAVGNHSLTHAQLSKTTGAALRSQVLDTDTLLRAVDDKRAPLFRFPYGARNAEGLQLLGEAGLKSIMWNIDSMDWADPVPESIVQRVLDQVNKEQRGIVLFHDIHDRAVKALPQILDRLIADGYQFAGWNGRDFSVARAPSATAAATVTTGYAKSWAIVIGIDDYARWPKLEYAANDAQAIANTLTGPLGFPSSQVIVLKNAEATRNNILAAFHDRLGDGRAQTNDRVFVFFAGHGATRRLASGRDLGYIIPVDSDPEHLANDAIAMSDLQNIAESLQAKHVLFVMDACYSGLGLTRGGPSSSAYLRENARRIARQMLTAGGADQQVADSGPNGHSVFTWVVLQALAGKGDLNGDGLITGTELAAYVAPAVSAVSAQTPAFGSLPGSQGGEFVFQVPSGEEFLTADTAQLSAAAIALNGRVDAVRPAAVSAGVPANAAPVTVKTLQGGEATLVLPTAVKSSDRQRAQLANERGLQLYKEKRYADAAEQFAEALKLRPDFALAANNLGFVYYRQERYAEAARWLENTLKIDPSRAVAYLNLGDAYAKAGDREKARKAYTTYLALQPQGAGAEQARAQLQTL

SEQ ID NO: 96 - protein, 656 aa, Xanthomonas campestris
MRHDAQPRQLTFRAVVLAIVLAVVLSAANAYLGLFAGLTIATAIPAAVVSMGVLRLLGGGTILENNIVQTGASAGSSIAAGVIFTIPALVIMGYWPDFKYWWVLGIAGMGGLLGVLFSVPLRRSMIVEDPLPFPEGKAAAEVLKAGENPGPGLKILAISGAIGALVKLGAASGLRVIPDTWAQATYLGSSRLVGYLGTNLSPALLGVGYIVGLNVGIVVLSGSILSWHIAIPLYQQFFMGSDPALAQSLVNAPAADAAFGIWGAKIRYLGVGAMLIGGVWTLFSLRKSLFSGIKSGFAAARKSGGGVVAETERDLPMKWMLVALVLCTLPLLGLYQAIVQQWHVSIPMTIIMIVAGFLFVSVSGYLAGLIGSSNNPVSGITISTILFASAVLVLLLGKSGLVPVGIGAAPLGAVAAIMIGAVVCCAAAVGGDNLQDLKAGYLVGATPWKQQLMLGIGAFSCALIMAPVLNLLAQAYGIGPATPQHPNSLGAPQATLMASVAKGLFGGQLPWSMIAIGAGVGAAIIALDEWLKKTGKRFRVPVLAAAIGIYLPLELMVPIFLGGLIAHLVERFHTIRADDEDGRDRVHRPGVLFAAGLITGEALMGIAIAVPIVLSSRADVLALPAALQLNQWVGLAILALVGWLLYRVGKRGEKAA

SEQ ID NO: 97 - protein, 271 aa, Xanthomonas campestris
MVFWRGCNVLLSLFVVDAFTCVRFKGNPAAVVPLQAWLPVPVMQAIAAENNLSETAFFVRDHAGFFDIRWFSPLKEVGFCGHATLASAYVIARQQLAPFPLRLRAPAVGEIGVEQVDEATFEMCFPNQLPTLVTQVPAPLLAALPIAPQAVYANAQAYIAVYADEQQVRAVVPDLAQMLALAPRDVAITAPGATHDLVSRYFWPANGGAEDPVTGSIHAALAPLWARTLGKTDLRALQASARGGELACRVTPAHVYVRGSAVQYLRGEITL

SEQ ID NO: 98 - protein, 427 aa, Xanthomonas campestris
MSWPVMESAMQSMWELDVSPTRFKAHFSLDPGVACLNHGMLGACPISVQQRQSTLRAQLERQPAAFVLRALPALLDTARNCLAEVIGADPQDLLLLPNVTTAMSAVLRSRIFAPGDQILTTDHAYLSCRNLLDFIARSTGAEVMVAPVKVPVHHPDAIVDAVLERVTARTRLAVLDHVTSPTAIVFPIARLVERLAALGIDTLVDGAHAPGMLPLDVIAIGAAYYAGDCHKWLCTPRGAGFLHVRRDRQDGLHPPVISRGYGDATPGRPRLHLEFDWLGTADPTALLCIPAAIDFLATLLPGGLPAVFARNHALVTQAAARLAQALPLARLAPDAMVGSMVAFQLPDAASDAPDDAAASLQRWLYDAHRIDVAVTPWPHRTNRTLRISAQVYNAAEDFMQLDAPLAQCLAPDDRQSNNQCRTTEVMR

SEQ ID NO: 99 - protein, 300 aa, Xanthomonas campestris
MTYPGYSFTPKRLDVRPGIAMSYLDEGPRDGEVVVMLHGNPSWSYLWRHLVSGLSDRYRCIVPDHIGMGLSDKPDDAPDAQPRYDYTLQSRVDDLDRLLQHLGITGPITLAVHDWGGMIGFGWALSHHEQVKRLVITNTAAFPLPPEKPMPWQIAMGRHWRLGEWFIRTFNAFSSGASWLGVSRRMPAAVRRAYVAPYDNWKNRISTIRFMQDIPLSSADQAWSLLERSAQALPSFADRPAFIAWGLRDICFDKHFLAGFRRALPQAEVMAFDDANHYVLEDKHEVLVPAIRAFLERNPL

SEQ ID NO: 100 - protein, 371 aa, Xanthomonas campestris
MSAQPNTTATRPDPGMQVTTFENPMGIDGFEFVEFAAPAGQAAQLHDYFRKMGFTAVLRHRSRPITVYRQGGVNFLLNEDPDSFAADFAAAHGPCACGFAIRFRTPADTVLQTVLGNGGEAVQNKPDTRAVPAPVVKGIGDCMLYLVDRYGEAGSIYDADYEAIEGADQHPAGFGLTFIDHLTHNLYFGNMQQWSDYYERLFNFREIRYFDIKGAKTGLVSKAMTAPDGIVRIPLNESSDPKSQINEYLDAYQGEGIQHIACFTDDIYTSVERMRAAGVTFLDTPDTYFDVVDLRIPDHGEDVERLRRNKILIDADVDTKQRKLLQIFTSNCIGPIFFEIIQRKGNEGFGEGNFQALFESIERDQMKRGVL

SEQ ID NO: 101 - protein, 434 aa, Xanthomonas campestris
MHNDQRYMTGFGNEFASEAVADTLPVGQNSPQRVAHGLYAEQLSGTAFTAPRGENRRSWLYRMRPAAVHGTFSLIEQSQFHNDFGHGPVPPDQLRWSPLPLPQTPTDFIDGLYTMAGNGSPEAMNGVAVHLYAANASMQDRFFYNADGELLLVPQLGRLRVHTELGMLELEPQQIGVIPRGVRFRVELRDGTARGYVCENFGGLLHLPDLGPIGSNGLANPRDFETPCAAFEQREGRFELVAKFQGHLWRADIGHSPLDVVAWHGNYAPYRYDLRRFNTIGSISFDHPDPSIFTVLTSPSDTHGTANMDFAIFPPRWLVAQHTFRPPWFHRNVASEFMGLVHGVYDAKADGFAPGGASLHNCMSGHGPDAATFDKASQADLSRPDVITETMAFMFETRAVLRPTAQSLHAPHRQGDYQQCWAGLRNAFQAPPAS

SEQ ID NO: 102 - protein, 298 aa, Xanthomonas campestris
MPRLSRLALLLSLTFAAGHAVAGPEEDQRARNAVRVLNEIMNIPEQAIPDKLLDEARAIVVIPDTLKAGLVIGGRRGHGLMSMKNADGSWSQPVFVKLTGGSIGFQAGVQSSDVVLVFRNDRSLDNIVNGKFTLGADAGVAAGPVGRNAAAATDGQLKAEIWSWSRARGLFAGVALDGAVLQIDDAADLNAYGGGATPRMIFEGRTNERPSTDVIAFRDRLEEVTYTARTNRSSDGDTPPPAPRPVSQSPAQAPATPPPPGNEASTMPIQPSATPPQQQGFQPVSDGEIRTESLDGNR

SEQ ID NO: 103 - protein, 352 aa, Xanthomonas campestris
MSSLATSFRAAVFAAGVTCIPAHVGATPDGNRTSAEAAQAVQPAQWRTVATHNTPHARHENSMVAIDDRLYLLGGRDDRPLEIFDTTTQRWSQGATPPLMVNHAQAVVSSGKLYVVGGFSGNYPEEASLTNMLIYDPKTDHWQVGPEIPTQRRRGAAGTVEHAGVLYLVGGNTRGHMSGYVPWLDAFDTRTQQWTQLPDAPHARDHFHAVVLDGKLYAAGGRRSAHESGNTLAQTISEVDVYDIAQRSWTVAPAMLPTPRAGTAAIARDGRLLVMGGESTRQVKAHEEVEAYDPRTARWTTLPTLPRGRHGTQAAAVKGDVYLAAGSANRGGGPELEDVLVLAKAQGTQSLD

SEQ ID NO: 104 - protein, 303 aa, Xanthomonas campestris
MRKGAALMVGVTGISGYNFANVLLADGWTVYGLARRPLSHEGVIPVAADLLDAEATKTALQGLPITHVFFCTWTRRDTERENVEANGAMMRHLCDGLSEAPLQHMALVTGTKHYLGAFENYGSGKAETPFRESEPRQPGENFYYTLEDLLFQHAEQHGFGWSVHRSHTMVGIANGSNAMNMGVTLAVYASLCKHTGQPFVFPGSQAQWNSLSDVTDAGLLGRQLAWAGLSPAARNQAFNTVNGDVFRWRWMWGEIAKFFGLEAAPCPQTPEPLEARLSDTAPAQWAELAAKHGLVEPDVNRLA

SEQ ID NO: 105 - protein, 275 aa, Xanthomonas campestris
MPSFKSFLLHRVSPVCLCVATGWGTVAMAQTVLPDATPVHAHETTQHDGHEWGYTGPEGAEHWAELAKENALCGNGQQNSPIDLKDAIDATLGKLQLDYGAVPLVVRNTGHSIQLDLHAGGTMRVGGKQYDALQLHFHHPSEHLLNGRRFPMEAHIVHQGPDGTLGVLAIFFETGKANPAFQRVLDAMPSDKNQTRQVADAVVRASDFLPPANQRSFYRYEGSLTTPPCSETVDWVVLSQPVQVSREQINAFERVYPFNARPLQPLDRRFLLKSH

SEQ ID NO: 106 - protein, 784 aa, Xanthomonas campestris
VMTQMANGADPRATDNDGNTPLHHAVRSSDPGVAALLRDAAAELDALNNEGHSPLAMACQVGNWRLAKFLLERGAQSEPAGGAPVLLAAAGTEEDDPAGVQLLLKHKARVDARDRQRRSALHEAALAGHGDIVEALLAAGANLEARDLLGRTPWLDAARQARSAVLERLVARKTDVLAIDGDGRNAVMLACAADTVSPALIRRLLELDVPADAQDMHGRRAVDVAAEAGRWAIVRLLDPAYPLPSAVSDAHGDGVGPTTQLPDRPPLVLLREGLQLGQRDGLVALARLCGPEELGGLLHDAHLALNADAVDWLLRHGADAEVRDACGDVPMFALLSRGVEAVPALQVLLRHGVSPAGAGGLTRLLSACVQHDAASRGLEQFALELLERGADPFAPSAAGDPPLSLAVRLGWLRLQQWLLEHGVDREARDSHGMTALHLATALGREASLKLLVKQGASPEARAADGQTPLGVALSIGRRDLADWLDWRIWSLPRRTLREADVPAAAMTGDTDAVRRLIDLGLPVDAVDAQGCTALLRAAGSGHLAVVTHLLGRGADPQRAANSGATPLSAAVSMRQTEIVAALLAAGAQIEHRLPGGVTVLMLACALGLPDIVARLLAAGADVHANDDQGLAALHCAALYGFTARDKIRLLALLDTLLLAGADPDYLAGGRVSPLLMLLGARAEPGTACDEQVVLAGVERLLDEDVALDVADQRGFGPLHLAALHGLPLLVQRLLRAGADSDRRDALNRTPREIAIMRGFIDVAGQFEPALPGVSSMARFLREGR

(19) United States
(12) Patent Application Publication
(10) Pub. No.: US 2005/0100892 A1
(43) Pub. Date: May 12, 2005

(54) Method of selecting genes for crop improvement

(76) Inventors: Terrance P. Shea JR., Somerville, MA (US); Steven C. Slater, Middleton, WI (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 10/200,545

(22) Filed: Jul. 22, 2002

(51) Int. Cl. C12Q 1/68; G06F 19/00; G01N 33/48; G01N 33/50; A01H 1/00; C12N 15/82

(52) U.S. Cl. 435/6; 800/288; 702/20

(57) ABSTRACT

Methods of selecting genes for crop improvement are provided. Also disclosed are polynucleotides and proteins selected using methods of the present invention. The disclosed polynucleotides find use in production of transgenic plants to provide plants, particularly crop plants, having improved properties.

### FIELD OF THE INVENTION

The present invention relates generally to the field of genomics. More particularly the invention relates to a method of comparing genomes to identify genes that may be useful in plants.

### BACKGROUND

As more and more genomes of organisms are sequenced, there is a need for tools to identify genes for crop improvement, e.g. genes involved in metabolic pathways in plants. Typically candidate genes for transgenic expression studies have been chosen using the bioinformatic approach of gene mining of genomes based on similarity, e.g. determined by BLAST, of gene sequences in one genome with those of known function or with family members of known function, as found in the scientific literature in another genome. Relying on gene mining alone to isolate genes of interest will result in genes of unknown function being ignored.

Methods of genome comparisons are currently used to study gene evolution and pathogenicity. Jeffrey G. Lawrence et al, Proc. Nat. Acad. Sci. USA, Vol. 95, pp. 9413-9417, 1998, report that some genes in an organism are acquired from a different organism (horizontal gene transfer) rather than evolved within the organism's genome through a series of point mutations. Lawrence et al. further report evaluation of the overall impact of horizontal gene transfer on the evolution of bacterial genomes. Others compare genomes of pathogenic versus non-pathogenic bacteria to determine the pathogenicity islands, aquired clusters of virulence genes, that have caused the bacteria to become pathogenic.

For medical applications, comparisons between genomes of humans and human pathogens are performed to find differences between the genomes to identify genes useful for antibiotic targeting. See, for example, Claire M. Fraser et al., Genomics, Vol 6, No. 5, September-October 2000 (available at “cdc.gov” web site).

In the field of plant genomics, genome comparison has applications in synteny and QTL mapping. It has also been used to identify polymorphisms and to analyze plant gene family evolution. What is needed in the art is a method to identify genes for crop improvement within non-plant genomes. What is further needed is a method to generate a list of candidate genes for evaluation which includes genes of unknown function.

### SUMMARY OF THE INVENTION

The present invention provides methods of selecting genes for crop improvement comprising:
a. comparing sequences of a subject organism to sequences of at least one plant species; and
b. identifying a candidate set comprising sequences of said subject organism that are similar to said sequences of at least one plant species.
   Also provided are methods with the additional steps of:
c. comparing sequences of said candidate set to sequences of a reference organism; and
d. creating a candidate subset which contains members of said candidate set which have a BLAST E value of E-9 or greater when compared to said sequences of a reference organism.
   Of particular interest are methods wherein “comparing” comprises a similarity search; “identifying” comprises selecting sequences of a microbial organism having a BLAST E value of at least E-9 when compared to a sequence of a plant species and/or “reference organism” is not a pathogen to said at least one plant species. Sequences used in methods of the present invention are preferably amino acid sequences.

In methods of the present invention, a subject organism is preferably a microbial organism, more preferably a plant pathogen. Plants useful in methods of the present invention include, but are not limited to, plants tolerant to said plant pathogen and plants sensitive to said plant pathogen.

The present invention provides candidate sets of microbial genes derived using methods the present invention. Of particular interest are candidate sets comprising:
a. one or more of the nucleic acid sequences selected from the group consisting of SEQ ID NO: 1 through SEQ ID NO: 53;
b. one or more nucleic acid molecules encoding proteins having a sequence selected from the group consisting of SEQ ID NO: 54 through SEQ ID NO: 106.
   The present invention further provides and contemplates transformed plants comprising at least one microbial gene selected from the group consisting of:
a. a nucleic acid molecule encoding a protein having an amino acid sequence of SEQ ID NO: 54 through SEQ ID NO: 106;
b. a nucleic acid molecule which encodes a functional homolog of a protein having a sequence of SEQ ID NO: 54 through SEQ ID NO: 106; and
c. a nucleic acid molecule encoding a polypeptide of at least 20 amino acid residues which are identical to a 20 amino acid segment in a protein having a sequence of SEQ ID NO: 54 through SEQ ID NO: 106, and which is a functional homolog of said protein.

### DETAILED DESCRIPTION

This invention provides a comparative genomics method to selectively determine genes or fragments thereof which encode polypeptides for crop improvement. Genes or polypeptides for crop improvement include but are not limited to proteins that control a plant pathway, genes responsible for an agronomically useful trait, and genes responsible for pest or disease resistance.

As used herein “gene” means a polynucleotide which encodes a polypeptide. Depending on the intended use, the polynucleotides of the present invention may be present in the form of DNA, such as cDNA or genomic DNA, or as RNA, for example mRNA. The term “polynucleotide” refers to a nucleic acid molecule.

As used herein, the term “polypeptide” means a chain of amino acid residues that are covalently linked by an amide linkage between the carboxyl group of one amino acid and the amino group of another. The term polypeptide can encompass whole proteins (i.e. a functional protein encoded by a particular gene), as well as fragments of proteins. Of particular interest are polypeptides of the present invention which represent whole proteins or a sufficient portion of the entire protein to impart the relevant biological activity of the protein. The term “protein” also includes molecules consisting of one or more polypeptide chains. Thus, a polypeptide of the present invention may also constitute an entire gene product, but only a portion of a functional oligomeric protein having multiple polypeptide chains.

Of particular interest in the present invention are polypeptides involved in one or more important biological properties in plants. Such polypeptides may be produced in transgenic plants to provide plants, particularly crop plants, having improved phenotypic properties and/or improved response to stressful environmental conditions. Crop plants of interest in the present invention include, but are not limited to soybean, cotton, canola, maize, wheat, sunflower, sorghum, alfalfa, barley, millet, rice, tobacco, fruit and vegetable crops, and turf grass. Other non-crop plants of interest include model plants such as Arabidopsis thaliana.

Of particular interest are uses of the disclosed polynucleotides to create transgenic plants for crop improvement. As used herein “crop improvement” comprises improved yield resulting from improved utilization of key biochemical compounds, such as nitrogen, phosphorous and carbohydrate, or resulting from improved responses to environmental stresses, such as cold, heat, drought, salt, and attack by pests or pathogens. Polynucleotides selected using the methods of the present invention may also be used to provide plants having improved growth and development, and ultimately increased yield, as the result of modified expression of plant growth regulators or modification of cell cycle or photosynthesis pathways. Other crop improvements include flavonoid content, seed oil and protein quantity and quality, herbicide tolerance and rate of homologous recombination.

As used herein “comparative genomics” means comparing nucleic acid sequences or amino acid sequences encoded by genes of a genome to nucleic acid or amino acid sequences of interest. The nucleic acid or amino acid sequences of interest may be another genome, a gene family, regulatory regions or a protein motif.

Comparisons of a large number of sequences are easily performed by search algorithms on a computer. A number of different search algorithms have been developed, one example of which are the suite of programs referred to as BLAST programs. There are five implementations of BLAST, three designed for nucleotide sequences queries (BLASTN, BLASTX, and TBLASTX) and two designed for protein sequence queries (BLASTP and TBLASTN) (Coulson, Trends in Biotechnology, 12: 76-80 (1994); Birren, etal., Genome Analysis, 1: 543-559 (1997)).

BLASTN takes a nucleotide sequence (the query sequence) and its reverse complement and searches them against a nucleotide sequence database. BLASTN was designed for speed, not maximum sensitivity, and may not find distantly related coding sequences. BLASTX takes a nucleotide sequence, translates it in three forward reading frames and three reverse complement reading frames, and then compares the six translations against a protein sequence database. BLASTX is useful for sensitive analysis of preliminary (single-pass) sequence data and is tolerant of sequencing errors (Gish and States, Nature Genetics, 3: 266-272 (1993), the entirety of which is herein incorporated by reference).

Given a coding nucleotide sequence and the protein it encodes, it is often preferable to use the protein as the query sequence to search a database because of the greatly increased sensitivity to detect more subtle relationships. This is due to the larger alphabet of proteins (20 amino acids) compared with the alphabet of nucleic acid sequences (4 bases), where it is far easier to obtain a match by chance. In addition, with nucleotide alignments, only a match (positive score) or a mismatch (negative score) is obtained, but with proteins, the presence of conservative amino acid substitutions can be taken into account. Here, a mismatch may yield a positive score if the non-identical residue has physical/chemical properties similar to the one it replaced. Various scoring matrices are used to supply the substitution scores of all possible amino acid pairs. A general purpose scoring system is the BLOSUM62 matrix (Henikoff and Henikoff, Proteins, 17: 49-61 (1993), the entirety of which is herein incorporated by reference), which is currently the default choice for BLAST programs. BLOSUM62 is tailored for alignments of moderately diverged sequences and thus may not yield the best results under all conditions. Altschul, J. Mol. Biol. 36: 290-300 (1993), the entirety of which is herein incorporated by reference, uses a combination of three matrices to cover all contingencies. This may improve sensitivity, but at the expense of slower searches. In practice, a single BLOSUM62 matrix is often used but others (PAM40 and PAM250) may be attempted when additional analysis is necessary. Low PAM matrices are directed at detecting very strong but localized sequence similarities, whereas high PAM matrices are directed at detecting long but weak alignments between very distantly related sequences.

Embodiments of this invention use sequence comparisons to narrow the number of genes selected for study, e.g. expression in plants. If more than one comparison is made, the comparisons may be done simultaneously or in any order.

Comparative genomics methods of this invention may be used to identify horizontal gene transfer events in dissimilar organisms, e.g. a microbial pathogen and a plant. As used herein “horizontal gene transfer” means genes that are introduced into an organism's genome by acquisition from another genome rather than through mutation and evolution of an existing gene. It is known that different organisms exhibit different codon preferences. Horizontally transferred gene sequences may be identified by looking at those with atypical base composition due to different codon usage of the transferred sequence versus the acquiring organism's codon usage (see Lawrence et al., Proc. Nat. Acad. Sci. USA, Vol. 95, pp. 9413-9417, 1998). However, even after horizontal gene transfer occurs, codon assimilation is likely over a period of time. This happens by point mutation and eventually the acquired gene looks so much like the acquiring organism's genes that one could not determine that its original host by nucleic acid sequence homology. Using comparitive genomics methods of the invention, horizontal gene transfer events from plants, even those genes that have undergone codon assimilation, will be identified.

Neither codon preference nor clustering of gene families is necessary to predict which genes are derived from plants using methods of the present invention. In preferred embodiments genes of unknown or uncharacterized function are selected for evaluation. Bioinformatic analysis such as homology based searches focus on genes of known function and clustering of gene families to identify previously unannotated homologs. Deriving a candidate gene set for study using homology and clustering ignores gene families and homologs in which no member is annotated and it allows such genes and homologs to remain unannotated. Since genes selected for study by methods of the present invention are chosen based on their homology to at least one plant gene, lack of annotation does not prevent, and may actually be a criterium for, including the gene in a candidate set for study.

Subject Organism

In embodiments of this invention, a comparison is made between gene or protein sequences of a subject organism and sequences of any number of plant genomes. As used herein “subject organism” refers to the organism from which gene candidates are sought. The genome of a subject organism contains genes in its genome which are likely to be of utility in modifying plants.

In preferred embodiments of the present invention, a subject organism is chosen based on its association with a plant (for example, a plant pathogen, symbiont, or epiphyte). The close association with the plant makes it likely to encode proteins capable of metabolizing plant-produced compounds. Thus, these genes are of potential utility for modifying plant metabolism. Preferably a subject organism is a non-plant organism, e.g. a microbial organism, virus or insect, more preferably a microbial organism, most preferably a microbial organism that is a plant pathogen.

Examples of preferred subject organisms from the species Agrobacterium and Xanthomonas are provided below in Tables 1 and 2, respectively, with plant host information and the disease they cause in that host. A person of ordinary skill in the art could identify plant pests or other desirable subject organisms from the literature.

TABLE 1

| Agrobacterium Species | Host | Disease |
|---|---|---|
| A. tumefaciens | dicotyledonous plants | crown gall |
| A. rhizogenes | dicotyledonous plants | hairy root |
| A. rubi | caneberry | cane gall |
| A. vitis | grape, chrysanthemum | crown gall |

TABLE 2

| Xanthomonas Species | Plant Host | Disease |
|---|---|---|
| X. axonopodis pv | soybean | Bacterial pustule |
| glycines | | |
| (X. campestris pv | | |
| glycines) | | |
| X. axonopodis pv | cassava | Bacterial blight |
| manihotis | | |
| (X. campestris pv | | |
| manihotis) | | |
| X. axonopodis pv | bean | Common bacterial |
| phaseoli | | blight |
| X. campestris pv | crucifers | Black rot |
| campestris | | |
| X. campestris pv | geranium | Leaf spot and Stem rot |
| pelargonii | | |
| X. citri | citrus | Citrus canker |
| X. oryzae pv oryzae | rice | Bacterial blight |
| X. translucens | small grains | Leaf streak and black |
| (X. campestris pv | | chaff |
| translucens) | | |
| X. vesicatoria | pepper, tomato | Bacterial spot |
| (X. campestris pv | | |
| vesicatoria) | | |

Genome Sequences

The genome sequence of the subject organism may be publically available or it may be obtained through direct sequencing of some or all of an organism. C. M. Fraser et al., Genomics, Vol 6, No. 5, September-October 2000 report that the sequences of close to 30 microbial genomes have been completed between 1995 and 2000, and the sequences of more than 100 genomes should be completed in the next few years. Microbial genomes publically available include Escherichia coli, Bacillus subtilis, Haemophilus influenzae, Helicobacter pylori, Mycobacterium tuberculosis and Saccaromyces cervisiae. PathoSeq database (a collection of proprietary data from Incyte's microbial genomes and publically available genomes; Incyte Genomics, Inc. Palo Alto, Calif.) now contains genomes of some plant pathogens e.g. Agrobacterium tumefaciens but not E.coli. In some cases genomic DNA is sequenced, for which it is useful to predict the most likely full-length open reading frame and the expected sequence of the full protein for each gene. Preferably a cDNA library is sequenced, ESTs are assembled into contigs and a proteome is generated by predicting proteins encoded by the genes and partial genes sequenced.

As used herein “sequences” means nucleic acid sequences and/or amino acid sequences, e.g. DNA, cognate RNA and protein sequences.

For incomplete genomes, sequence comparisons and algorithms are employed to predict the most like open reading frames (ORFs) and the expected sequence of each full length protein and the gene encoding the protein. Prediction of coding regions, ORFs and proteins encoded are easily performed using similarity search algorithms. A full length gene is often not required for encoded protein prediction. Sequence comparisons can be undertaken by determining the similarity of the test or query sequence with sequences in publicly available or propriety databases (“similarity analysis”) or by searching for certain motifs (“intrinsic sequence analysis”), e.g. cis elements (Coulson, Trends in Biotechnology, 12: 76-80 (1994), the entirety of which is herein incorporated by reference; Birren, et al., Genome Analysis, 1: 543-559 (1997), the entirety of which is herein incorporated by reference).

Similarity analysis includes database search and alignment. Examples of public databases include the DNA Database of Japan (DDBJ)(available at the website “ddbj.nig.ac.jp”); the non-redundant protein (i.e., nr-aa) database maintained by the National Center for Biotechnology Information as part of GenBank and available at the web site for “ncbi.nln.nih.gov”; and the European Molecular Biology Laboratory Nucleic Acid Sequence Database (EMBL) (available at the web site “ebi.ac.uk”). As stated above, a number of search algorithms have been developed, one example of which are the suite of programs referred to as BLAST programs. BLASTN and BLASTX may be used in concert for analyzing EST data (Coulson, Trends in Biotechnology, 12: 76-80 (1994); Birren, et al., Genome Analysis, 1: 543-559 (1997).

Homologues in other organisms are available that can be used to predict likely full length genes of a genome and the encoded proteins. Multiple alignments are performed to study similarities and differences in a group of related sequences. CLUSTAL W is a multiple sequence alignment package available that performs progressive multiple sequence alignments based on the method of Feng and Doolittle, J. Mol. Evol. 25: 351-360 (1987), the entirety of which is herein incorporated by reference. Each pair of sequences is aligned and the distance between each pair is calculated; from this distance matrix, a guide tree is calculated, and all of the sequences are progressively aligned based on this tree. A feature of the program is its sensitivity to the effect of gaps on the alignment; gap penalties are varied to encourage the insertion of gaps in probable loop regions instead of in the middle of structured regions. Users can specify gap penalties, choose between a number of scoring matrices, or supply their own scoring matrix for both the pairwise alignments and the multiple alignments. CLUSTAL W for UNIX and VMS systems is available at: ftp.ebi.ac.uk. Another program is MACAW (Schuler et al., Proteins, Struct. Func. Genet, 9:180-190 (1991), the entirety of which is herein incorporated by reference, for which both Macintosh and Microsoft Windows versions are available. MACAW uses a graphical interface, provides a choice of several alignment algorithms, and is available by anonymous ftp at: ncbi.nlm.nih.gov (directory/pub/macaw).

Sequence motifs are derived from multiple alignments and can be used to examine individual sequences or an entire database for subtle patterns. With motifs, it is sometimes possible to detect distant relationships that may not be demonstrable based on comparisons of primary sequences alone. Currently, the largest collection of sequence motifs in the world is PROSITE (Bairoch and Bucher, Nucleic Acid Research, 22: 3583-3589 (1994), the entirety of which is herein incorporated by reference.) PROSITE may be accessed via either the ExPASy server on the World Wide Web or anonymous ftp site. Many commercial sequence analysis packages also provide search programs that use PROSITE data.

A resource for searching protein motifs is the BLOCKS E-mail server developed by S. Henikoff, Trends Biochem Sci., 18:267-268 (1993), the entirety of which is herein incorporated by reference; Henikoff and Henikoff, Nucleic Acid Research, 19:6565-6572 (1991), the entirety of which is herein incorporated by reference; Henikoff and Henikoff, Proteins, 17: 49-61 (1993). BLOCKS searches a protein or nucleotide sequence against a database of protein motifs or “blocks.” Blocks are defined as short, ungapped multiple alignments that represent highly conserved protein patterns. The blocks themselves are derived from entries in PROSITE as well as other sources. Either a protein or nucleotide query can be submitted to the BLOCKS server; if a nucleotide sequence is submitted, the sequence is translated in all six reading frames and motifs are sought in these conceptual translations. Once the search is completed, the server will return a ranked list of significant matches, along with an alignment of the query sequence to the matched BLOCKS entries.

Conserved protein domains can be represented by two-dimensional matrices, which measure either the frequency or probability of the occurrences of each amino acid residue and deletions or insertions in each position of the domain. This type of model, when used to search against protein databases, is sensitive and usually yields more accurate results than simple motif searches. Two popular implementations of this approach are profile searches (such as GCG program ProfileSearch) and Hidden Markov Models (HMMs)(Krough et al., J. Mol. Biol. 235:1501-1531 (1994); Eddy, Current Opinion in Structural Biology 6:361-365 (1996), both of which are herein incorporated by reference in their entirety). In both cases, a large number of common protein domains have been converted into profiles, as present in the PROSITE library, or HHM models, as in the Pfam protein domain library (Sonnhammer et al., Proteins 28:405-420 (1997), the entirety of which is herein incorporated by reference). Pfam contains more than 500 HMM models for enzymes, transcription factors, signal transduction molecules, and structural proteins. Protein databases can be queried with these profiles or HMM models, which will identify proteins containing the domain of interest. For example, HMMSW or HMMFS, two programs in a public domain package called HMMER (Sonnhammer et al., Proteins 28:405-420 (1997)) can be used.

PROSITE and BLOCKS represent collected families of protein motifs. Thus, searching these databases entails submitting a single sequence to determine whether or not that sequence is similar to the members of an established family. Programs working in the opposite direction compare a collection of sequences with individual entries in the protein databases. An example of such a program is the Motif Search Tool, or MoST (Tatusov et al. Proc. Natl. Acad. Sci. 91: 12091-12095 (1994), the entirety of which is herein incorporated by reference.) On the basis of an aligned set of input sequences, a weight matrix is calculated by using one of four methods (selected by the user); a weight matrix is simply a representation, position by position in an alignment, of how likely a particular amino acid will appear. The calculated weight matrix is then used to search the databases. To increase sensitivity, newly found sequences are added to the original data set, the weight matrix is recalculated, and the search is performed again. This procedure continues until no new sequences are found.

Once a subject organism's genome is obtained it is compared to plant sequences. Comparison of the genome of a subject organism and at least one plant may be conducted at the DNA or protein level. In preferred embodiments, the amino acid sequences of the encoded proteins of a subjest organism are compared to plant amino acid sequences to avoid missing matches due to different codon preferences of dissimilar organisms.

Plant sequences used in a comparison with a subject organism may be genes of interest, whole genomes or gene families across several plant species. Plant species of interest include Arabidopsis thaliana and crop plants, such as soybean, cotton, canola, maize, wheat, sunflower, sorghum, alfalfa, barley, millet, rice, tobacco, fruit and vegetable crops, and turf grass.

Proprietary microbial and plant genomes useful for this invention include: Xanthomonas campestris pv campestris, disclosed in U.S. Ser. No. 09/703,708, filed Nov. 2, 2000, and Agrobacterium tumefaciens, disclosed in U.S. Ser. No. 09/514,000, filed Feb. 23, 2000, and genomic rice database (available at Monsanto's rice-research.org Internet Web site), incorporated herein by reference. Maize and soybean EST databases used in Example 1 to illustrate the invention are unpublished. A candidate set resulting from such a comparison contains the genes and/or the encoded proteins of a subject organism that are identical or homologous to plant genes or proteins.

Candidate Set

As used herein, a “candidate set” is the set of genes remaining after at least one comparison between sets of nucleic acid and/or amino acid sequences is performed. In the simplest case a candidate set results from a comparison of the subject genome to sequences from at least one plant and comprises the set of subject genes which are similar to plant genes. As used herein, “similar” means having a BLAST E value greater than or equal to E-9.

E values describe the probability that matches occur by chance. The expectation E (range 0 to infinity) calculated for an alignment between the query sequence and a database sequence can be extrapolated to an expectation over the entire database search, by converting the pairwise expectation to a probability (range 0-1) and multiplying the result by the ratio of the entire database size (expressed in residues) to the length of the matching database sequence. In detail:
E_database=(1−exp(−E))D/d

where D is the size of the database; d is the length of the matching database sequence; and the quantity (1−exp(−E)) is the probability, P, corresponding to the expectation E for the pairwise sequence comparison. E-9 is the same value as 1.00E-9 and 10.0E-10.

Embodiments of this invention use sequence comparisons to narrow the number of subject organism genes included in a candidate set to form a “candidate subset”. If more than one comparison is made, the comparisons may be done simultaneously or in any order to derive the candidate subset. One can create smaller candidate subsets by performing more comparisons.

For example, a candidate subset may contain sequences of a subject organism common to a monocot plant and not found in a dicot plant when comparisons of the subject organism to monocots and dicots are performed to include homologs of monocots and exclude homologs of dicots. Likewise, a candidate subset may comprise sequences from a subject organism found in plants pathogenized by the subject organism by creating a set of subject organism sequences common to plants pathogenized by the subject organism and excluding sequences also found in plants that are not pathogenized by the subject organism.

A candidate subset may also be formed by subtracting sequences homologous to those of a reference organism from the sequences of a subject organism.

Reference Organism

As used herein, “reference organism” means an organism more related to the subject organism than to the at least one plant. The reference organism is preferably not pathogenic to the same plant(s) that the subject organism pathogenizes. The reference organism, however may be a plant pathogen of a different plant species or ecotype or cultivar. The reference organism is preferably closely related (evolutionarily) to the subject organism. For preferred embodiments of the present invention, E. coli is a reference organism for each of the subject organisms Agrobacterium tumefaciens and Xanthomonas campestris pv campestris. The genomes of reference organisms, as for the subject organism, are preferably mostly or fully sequenced.

In a comparison between the genomes of the subject organism and reference organism, the resulting candidate set contains genes represented in the subject organism and not in the reference organism. Likewise, in a comparison between the set of sequences of a subject organism similar to sequences of at least one plant and the sequences of a reference organism, sequences in common are excluded from the resulting candidate set.

A comparison of subject and reference organisms would not be performed in all cases. If housekeeping genes such as glycolysis genes are desired, a single comparison of subject and plant would be performed. Genes involved in metabolic pathways, however, may be found more easily if one narrows the set of the subject organism genes common to plants, by subtracting genes also found in a reference organism. This reduces the number of genes in the candidate subset as compared to the candidate set by 20 to 50 fold.

Express Candidate Set In Plants

A preferred aspect of this invention is to look at the candidate set derived from computer-driven comparisons “by hand” as described in Example 2. The researcher looks at each sequence to keep, for example proteins of unknown function and commercially important proteins such as enzymes used in amino acid biosynthesis, metabolism, transcription, translation, RNA processing, nucleic acid and a protein degradation, protein modification and DNA replication, restriction, modification, recombination and repair, and to eliminate sequences that are homologous to structural proteins. Sequences may be poor quality, e.g. contain many n's. Since homology based on sequencing errors or ambiguity is undesirable, such hits may be discarded.

Once the candidate set has been determined, each gene may be analyzed to confirm that it encodes a protein for crop improvement. Genes from the candidate set may be transferred into a plant cell and the plant cell regenerated into a whole, fertile or sterile plant. Genes identified by the methods of this invention may be transferred into either monocotyledons and dicotyledons including but not limited to crop plants and model plants.

A variety of methods can be used to generate stable transgenic plants. These include particle gun bombardment (Fromm M. E. et al., 1990, Bio/Technology 8:833-839), electroporation of protoplasts (Rhodes, C. A. et al., 1989, Science 240:204-207; Shimamoto K. et al., 1989, Nature 338:274-276)), treatment of protoplasts with polyethylene glycol (Datta S. K. et al., 1990, Bio/Technology, 8:736-740), microinjection (Neuhaus, G. et al., 1987, Theoretical and Applied Genetics, 75:30-36), immersion of seeds in a DNA solution (Ledoux, L. et al., Nature, 249:17-21), and transformation with T-DNA of A. tumefaciens (Valvekens, D. et al., 1988, PNAS, 85:5536-5540; Komari T., 1989, Plant Science, 60:223-229). In many plant species, A. tumefaciens-mediated transformation is the most efficient and easiest of these methods to use. TDNA transfer generally produces the greatest number of transformed plants with the fewest multi-copy insertions, rearrangements, and other undesirable events.

Many different methods for generating transgenic plants using A. tumefaciens have been described. In general, these methods rely on a “disarmed” A. tumefaciens strain that is incapable of inducing tumors, and a binary plasmid transfer system. The disarmed strain has the oncogenic genes of the T-DNA deleted. A binary plasmid transfer system consists of one plasmid with 23-25 base pair T-DNA left and right border sequences, between which a gene for a selectable marker (e.g., an herbicide resistance gene) and other desired genetic elements are cloned and a second plasmid which encodes the A. tumefaciens genes necessary for effecting the transfer of the DNA between the border sequences in the first plasmid. When plant tissue is exposed to Agrobacterium carrying the two plasmids, the DNA between the left and right border repeats is transferred into the plant cells, transformed cells are identified using the selectable marker, and whole plants are regenerated from the transformed tissue. Plant tissue types that have been reported to be transformed using variations of this method include: cultured protoplasts (Komari, T., 1989, Plant Science, 60:223-229), leaf disks (Lloyd, A. M. et al., 1986, Science 234:464466), shoot apices (Gould, J., et al., 1991, Plant Physiology, 95:426-434), root segments (Valvekens, D. et al., 1988, PNAS, 85:5536-5540), tuber disks (Jin, S. et al., 1987, Journal of Bacteriology, 169: 44174425), and embryos (Gordon-Kamm W., et al., 1990, Plant Cell, 2:603-618).

In the case of Arabidopsis thaliana it is possible to perform in planta germline transformation (Katavic B., et al., 1994, Molecular and General Genetics, 245:363-370; (Clough, S. and Bent, A., 1998, Plant Journal, 16:735-743). In the simplest of these methods, flowering Arabidopsis plants are dipped into a culture of Agrobacterium such as that described in the previous paragraph. Among the seeds produced from these plants, 1% or more have integration of T-DNA into the genome.

Monocot plants have generally been more difficult to transform with Agrobacterium than dicot plants. However, “supervirulent” strains of Agrobacterium with increased expression of the virB and virG genes have been reported to transform monocot plants with increased efficiency (Komari T. et al., 1986, Journal of Bacteriology, 166:88-94; Jin S., et al., 1987, Journal of Bacteriology, 169:417-425).

Most T-DNA insertion events are due to illegitimate recombination events and are targeted to random sites in the genome. However, given sufficient homology between the transferred DNA and genomic sequence, it has been reported that integration of T-DNA by homologous recombination may be obtained at a very low frequency. Even with long stretches of DNA homology, the frequency of integration by homologous recombination relative to integration by illegitimate recombination is roughly 1:1000 (Miao, Z. and Lam, E., 1995, Plant Journal, 7:359-365; Kempin S. A. et al., 1997, 389:802-803).

Genes may be transferred into a plant cell by the use of a DNA vector or construct designed for such a purpose. Vectors have been engineered for transformation of large DNA inserts into plant genomes. Binary bacterial artificial chromosomes have been designed to replicate in both E. coli and A. tumefaciens and have all of the features required for transferring large inserts of DNA into plant chromosomes. BAC vectors, e.g., a pBACwich, have been developed to achieve site-directed integration of DNA into a genome.

A construct or vector may also include a plant promoter to express the protein or protein fragment of choice. A number of promoters which are active in plant cells have been described in the literature. These include the nopaline synthase (NOS) promoter, the octopine synthase (OCS) promoter, a cauliflower mosaic virus promoter such as the CaMV 19S promoter and the CaMV 35S promoter, the figwort mosaic virus 35S promoter, the light-inducible promoter from the small subunit of ribulose-1,5-bisphosphate carboxylase (ssRUBISCO), the Adh promoter, the sucrose synthase promoter, the R gene complex promoter, and the chlorophyll a/b binding protein gene promoter. For the purpose of expression in source tissues of the plant, such as the leaf, seed, root or stem, it is preferred that the promoters utilized in the present invention have relatively high expression in these specific tissues. For this purpose, one may choose from a number of promoters for genes with tissue- or cell-specific or -enhanced expression. Examples of such promoters reported in the literature include the chloroplast glutamine synthetase GS2 promoter from pea, the chloroplast fructose-1,6-biphosphatase (FBPase) promoter from wheat, the nuclear photosynthetic ST-LS1 promoter from potato, the phenylalanine ammonia-lyase (PAL) promoter and the chalcone synthase (CHS) promoter from Arabidopsis thaliana. Also reported to be active in photosynthetically active tissues are the ribulose-1,5-bisphosphate carboxylase (RbcS) promoter from eastern larch (Larix laricina), the promoter for the cab gene, cab6, from pine, the promoter for the Cab-1 gene from wheat, the promoter for the CAB-1 gene from spinach, the promoter for the cab1R gene from rice, the pyruvate, orthophosphate dikinase (PPDK) promoter from Zea mays, the promoter for the tobacco Lhcb gene, the Arabidopsis thaliana SUC2 sucrose-H+ symporter promoter, and the promoter for the thylacoid membrane proteins from spinach (psaD, psaF, psaE, PC, FNR, atpC, atpD, cab, rbcS). Other promoters for the chlorophyl a/b-binding proteins may also be utilized in the present invention, such as the promoters for LhcB gene and PsbP gene from white mustard (Sinapis alba). Additional promoters that may be utilized are described, for example, in U.S. Pat. Nos. 5,378,619; 5,391,725; 5,428,147; 5,447,858; 5,608,144; 5,608,144; 5,614,399; 5,633,441; 5,633,435 and 4,633,436.

Constructs or vectors may also include, with the coding region of interest, a nucleic acid sequence that acts, in whole or in part, to terminate transcription of that region. For example, such sequences have been isolated including the Tr7 3′ sequence and the nos 3′ sequence or the like. It is understood that one or more sequences of the present invention that act to terminate transcription may be used.

A vector or construct may also include other regulatory elements or selectable markers. Selectable markers may also be used to select for plants or plant cells that contain the exogenous genetic material. Examples of such include, but are not limited to, a neo gene which codes for kanamycin resistance and can be selected for using kanamycin, G418, etc.; a bar gene which codes for bialaphos resistance; a mutant EPSP synthase gene which encodes glyphosate resistance; a nitrilase gene which confers resistance to bromoxynil, a mutant acetolactate synthase gene (ALS) which confers imidazolinone or sulphonylurea resistance; and a methotrexate resistant DHFR gene.

A vector or construct may also include a screenable marker to monitor expression. Exemplary screenable markers include a b-glucuronidase or uidA gene (GUS), an R-locus gene, which encodes a product that regulates the production of anthocyanin pigments (red color) in plant tissues; a b-lactamase gene, a gene which encodes an enzyme for which various chromogenic substrates are known (e.g., PADAC, a chromogenic cephalosporin); a luciferase gene, a xylE gene which encodes a catechol dioxygenase that can convert chromogenic catechols; an a-amylase gene, a tyrosinase gene which encodes an enzyme capable of oxidizing tyrosine to DOPA and dopaquinone which in turn condenses to melanin; an a-galactosidase, which will turn a chromogenic a-galactose substrate. Included within the terms “selectable or screenable marker genes” are also genes which encode a secretable marker whose secretion can be detected as a means of identifying or selecting for transformed cells. Examples include markers which encode a secretable antigen that can be identified by antibody interaction, or even secretable enzymes which can be detected catalytically. Secretable proteins fall into a number of classes, including small, diffusible proteins detectable, e.g., by ELISA, small active enzymes detectable in extracellular solution (e.g, a-amylase, b-lactamase, phosphinothricin transferase), or proteins which are inserted or trapped in the cell wall (such as proteins which include a leader sequence such as that found in the expression unit of extension or tobacco PR-S). Other possible selectable and/or screenable marker genes will be apparent to those of skill in the art.

Thus, any of the genes found using the method of the present invention may be introduced into a plant cell in a permanent or transient manner in combination with other genetic elements such as vectors, promoters enhancers etc. Further any of the nucleic acid molecules encoding an A. tumefaciens or X. campestris protein may be introduced into a plant cell in a manner that allows for over expression of the protein or fragment thereof encoded by the nucleic acid molecule.

Phenotype Measurement

Expression of the polynucleotides and the concomitant production of polypeptides encoded by the polynucleotides is of interest for production of transgenic plants having improved properties, particularly, improved properties which result in crop plant yield improvement. Expression of polypeptides of the present invention in plant cells may be evaluated by specifically identifying the protein products of the introduced genes or evaluating the phenotypic changes brought about by their expression. It is noted that when the polypeptide being produced in a transgenic plant is native to the target plant species, quantitative analyses comparing the transformed plant to wild type plants may be required to demonstrate increased expression of the polypeptide of this invention.

Assays for the production and identification of specific proteins make use of various physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures may also be used to identify the expression of proteins by their functionality, particularly where the expressed protein is an enzyme capable of catalyzing chemical reactions involving specific substrates and products. These reactions may be measured, for example in plant extracts, by providing and quantifying the loss of substrates or the generation of products of the reactions by physical and/or chemical procedures.

In many cases, the expression of a gene product is determined by evaluating the phenotypic results of its expression. Such evaluations may be simply as visual observations, or may involve assays. Such assays may take many forms including but not limited to analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry. Morphological changes may include greater stature or thicker stalks.

The following examples are illustrative only. It is not intended that the present invention be limited to the illustrative embodiments.

### EXAMPLE 1

This example serves to illustrate comparative genomics methods used to select a candidate subset. First the subject organisms were compared to plants to create a candidate set and then the candidate set was narrowed by excluding sequences similar to sequences in a reference organism to create a candidate subset.

Two subject organisms were selected, Agrobacterium tumefaciens and Xanthomonas campestris. The genome sequences of each subject organism used are the subject of pending patent applications, “Xanthomonas campestris genome sequences and uses thereof,” Ser. No. 09/703,708, filed Nov. 2, 2000 and “Agrobacterium tumefaciens genome sequences and uses thereof,” Ser. No. 09/803,110, filed Mar. 12, 2001 both of which are incorporated herein in their entirety. The peptides encoded by genes and partial genes of both subject organisms were determined using a combination of homology-based and predictive programs. The predicted amino acid sequences are the most probable translations for the identified start and stop signals, and the biases in codon usage seen in Agrobacterium genes.

Comparison of protein sequences from both subject organisms to either nucleic acid or amino acid plant sequence databases was performed using either TBLASTN or BLASTP. Protein sequences from A. tumefaciens and X. campestris were compared to nucleic acid sequences (maize ESTs, soybean ESTs and rice genomic DNA sequences) using TBLASTN version 2.0.8 [Jan-05-1999]. Comparison of protein sequences from both subject organisms to nr-aa (the non-redundant protein database available at the GenBank website “ncbi.nlm.nih.gov”) and an Arabidopsis thaliana protein database comprised of public and Monsanto proprietary Arabidopsis proteins were performed using BLASTP version 2.0.8 [Jan-05-1999]. Subject organism protein sequences that had a BLAST “E value” greater than E-9 using default parameters to any sequence of the maize, soybean, rice or nr-aa database were included in the candidate set of plant-like subject organism sequences.

Next, the candidate set, comprising the subset of plant-like subject organism protein sequences, were compared to an E. coli protein database (used as the reference organism). The homology-based method for the comparison was BLASTP. Sequences from the candidate set that matched a sequence from E coli with a BLASTP E value greater than E-9 were not included in the candidate subset. Since structural genes of the microbial subject organisms are likely to be similar to structural genes in E. coil, this step removes sequences of such genes from the candidate set.

### EXAMPLE 2

This example serves to illustrate reviewing members of the candidate subset “by hand.” In this step, the sequence quality and annotation of each member is examined. If the sequence quality is poor, e.g. single reads of low complexity DNA, the gene prediction may be incorrect. Any gene prediction which looks to be a bad call is removed from the candidate subset. If there are genes that are not of interest, e.g. genes with both publically known sequence and well characterized effect in plants, they can also be removed in this step. In this example no sequence was excluded based on it's annotation. Structural proteins had already been removed by comparison to E. coli as a reference organism. One protein, annotated as as actin-interacting protein, was kept because it is very unusual to find a gene like that in a bacterium.

Gene sequences remaining in the candidate subset are provided as SEQ ID NOs 1-53 and their corresponding protein sequences are provided as SEQ ID NOs 54-106. All are shown with annotation and BLAST E values in Table 3.

TABLE 3

| Seq | | coding | Pep | | | |
|---|---|---|---|---|---|---|
| Num | Seq_ID | seq | Num | ATHAL_TOP | ATHAL_TOP_EVAL | NRAA_TOP |
| 1 | AGR40U_733 | 121-1146 | 54 | gi\|6648045\|sp | 2.00E−36 | g7303243 (AE003816) |
| | | | | \|O80543\|YJ83_{—} | | CG8067 gene product |
| | | | | ARATH HYPOTHETICAL | | [Drosophila |
| | | | | PROTEIN T22J18.3 | | melanogaster] |
| 2 | AGR40U_397 | 121-564 | 55 | gi\|3810594\|gb | 5.00E−13 | g3925819 (AB020211) |
| | | | | \|AAC69376.1\| | | unnamed protein |
| | | | | (AC005398) | | product |
| | | | | hypothetical protein | | [Roseobacter |
| | | | | | | denitrificans] |
| 3 | AGR40U_1084 | 121-819 | 56 | gi\|7484845\| | 2.00E−31 | g1339949 (D85230) |
| | | | | pir\|\|T10618 | | hypothetical |
| | | | | cell-cell signaling | | protein |
| | | | | protein csgA homolog | | [Plectonema |
| | | | | F21C20.110 | | boryanum] |
| 4 | AGR40U_1249 | 121-972 | 57 | gi\|7485974\| | 1.00E−18 | g7485974 |
| | | | | pir\|\|T05561 | | hypothetical protein |
| | | | | hypothetical protein | | F22K18.70 - |
| | | | | F22K18.70 | | Arabidopsis thaliana |
| 5 | AGR40U_1476 | 121-1086 | 58 | gi\|7671457\|emb | 7.00E−20 | g7671457 (AL353995) |
| | | | | \|CAB89397.1\| | | putative protein |
| | | | | (AL353995) | | [Arabidopsis |
| | | | | putative protein | | thaliana] |
| 6 | AGR40U_1775 | 121-1212 | 59 | gi\|6759445\|emb | 3.00E−26 | g7469439 |
| | | | | \|CAB69850.1\| | | hypothetical |
| | | | | (AL137189) | | protein - |
| | | | | putative protein | | Synechocystis sp. |
| | | | | | | (strain PCC |
| | | | | | | 6803) |
| | | | | | | [Synechocystis sp.] |
| 7 | AGR40U_2325 | 121-678 | 60 | g6175185\| | 2.00E−12 | “g4502089 |
| | | | | AAF04911.1\| | | ankyrin 1, |
| | | | | AC011437_26 | | erythrocytic |
| | | | | (AC011437) | | [Homo sapiens]” |
| | | | | ankyrin-like protein | | |
| 8 | AGR40U_2674 | 121-966 | 61 | g\|6630461\| | 1.00E−22 | g6630461 (AC007190) |
| | | | | AAF19549.1\| | | F23N19.9 |
| | | | | AC007190_17 | | [Arabidopsis |
| | | | | (AC007190) F23N19.9 | | thaliana] |
| 9 | AGR40U_2749 | 121-1023 | 62 | gi\|8885554\| | 7.00E−21 | “g7225260 |
| | | | | dbj\|BAA97484.1 | | (AE002362) |
| | | | | (AB025604) | | hydrolase, putative |
| | | | | ripening-related | | [Neisseria |
| | | | | protein-like/ | | meningitidis]” |
| | | | | hydrolase-like | | |
| 10 | AGR40U_2784 | 121-993 | 63 | | | |
| 11 | AGR40U_3537 | 121-1068 | 64 | MJC20.7 | 1.00E−22 | g6321004 |
| | | | | MJC20.7 - | | Yer156cp |
| | | | | unknown protein | | [Saccharomyces |
| | | | | | | cerevisiae] |
| 12 | AGR40U_4011 | 121-1920 | 65 | MLE2.5 MLE2.5 | 3.00E−73 | g7445199 |
| | | | | | | hypothetical protein |
| | | | | | | RP441 - |
| | | | | | | Rickettsia prowazekii |
| | | | | | | [Rickettsia |
| | | | | | | prowazekii] |
| 13 | XCCU3_570 | 121-2496 | 66 | g7630051 | 2.00E−81 | g5881940 (AL117387) |
| | | | | (AL353013) | | putative secreted |
| | | | | putative protein | | protein |
| | | | | [Arabidopsis | | [Streptomyces |
| | | | | thaliana] | | coelicolor A3(2)] |
| | | | | @hypothetical | | |
| | | | | sT49909 sprotein | | |
| | | | | T24H18.120 - Arabidopsis | | |
| | | | | thaliana; >g7630051\| | | |
| | | | | emb\|CAB88259.1\| | | |
| | | | | (AL353013) | | |
| | | | | putative protein | | |
| | | | | [Arabidopsis thaliana] | | |
| | | | | T24H18.12 | | |
| | | | | T24H18_120 [ ] | | |
| 14 | XCCU3_655 | 121-1002 | 67 | g6437556 (AC011623) | 8.00E−32 | g6437556 (AC011623) |
| | | | | unknown protein | | unknown protein |
| | | | | [Arabidopsis thaliana] | | [Arabidopsis |
| | | | | @(AC011623) | | thaliana] |
| | | | | sg6437556\|gb\| | | |
| | | | | AAF08583.1\| | | |
| | | | | AC011623_16 | | |
| | | | | sunknown protein | | |
| | | | | [Arabidopsis thaliana] | | |
| | | | | F24P17.24 [ ] | | |
| 15 | XCCU3_307 | 121-1011 | 68 | g7801672 (AL356014) | 6.00E−28 | g4521959 (J05278) |
| | | | | pirin-like | | pirin |
| | | | | protein | | [Ralstonia sp. |
| | | | | [Arabidopsis thaliana] | | CH34] |
| | | | | @pirin-like | | |
| | | | | sT48990 sprotein - | | |
| | | | | Arabidopsis | | |
| | | | | thaliana; >g7801672\| | | |
| | | | | emb\|CAB91592.1\| | | |
| | | | | (AL356014) | | |
| | | | | pirin-like protein | | |
| | | | | [Arabidopsis thaliana] | | |
| | | | | F25L23.8 | | |
| | | | | F25L23_80 [ ] | | |
| 16 | XCCU3_619 | 121-1257 | 69 | g6759445 (AL137189) | 3.00E−22 | g7469439 |
| | | | | putative protein | | hypothetical |
| | | | | [Arabidopsis thaliana] | | protein - |
| | | | | @hypothetical | | Synechocystis sp. |
| | | | | sT45962 sprotein | | (strain PCC 6803) |
| | | | | F7J8.200 - Arabidopsis | | [Synechocystis sp.] |
| | | | | thaliana; >g6759445\| | | |
| | | | | emb\|CAB69850.1\| | | |
| | | | | (AL137189) | | |
| | | | | putative protein | | |
| | | | | [Arabidopsis thaliana] | | |
| | | | | F7J8.20 | | |
| | | | | putative protein [ ] | | |
| 17 | 3apr_xan10_{—} | 121-1596 | 70 | g7452421 | 7.00E−43 | g5912520 (AL117669) |
| | gm1302_{—} | | | hypothetical protein | | putative large |
| | 3apr_xan10_{—} | | | F10M10.30 - | | secreted protein |
| | gm1303 | | | Arabidopsis thaliana | | [Streptomyces |
| | | | | [ ] | | coelicolor A3(2)] |
| 18 | XCCU3_678 | 121-1059 | 71 | K13E13.12 | 2.00E−29 | g7594580 (AJ288956) |
| | | | | | | hypothetical protein |
| | | | | | | [Arabidopsis |
| | | | | | | thaliana] |
| 19 | XCCU3_731 | 121-1155 | 72 | g8346554 (AL357612) | 1.00E−32 | g7464135 |
| | | | | putative protein | | hypothetical protein |
| | | | | [Arabidopsis thaliana] | | HP0049 - |
| | | | | @(AL357612) | | Helicobacter pylori |
| | | | | sg8346554\|emb | | (strain 26695) |
| | | | | \|CAB93718.1\| | | [Helicobacter |
| | | | | sputative protein | | pylori 26695] |
| | | | | [Arabidopsis thaliana] | | |
| | | | | T22D6.11 | | |
| | | | | T22D6_110 [ ] | | |
| 20 | XCCU3_737 | 121-2067 | 73 | g7573480 (AL163832) | 8.00E−20 | g7478865 |
| | | | | putative protein | | probable regulatory |
| | | | | [Arabidopsis thaliana] | | protein - |
| | | | | @hypothetical | | Mycobacterium |
| | | | | sT49197 sprotein | | tuberculosis |
| | | | | F27K19.30 - Arabidopsis | | (strain H37RV) |
| | | | | thaliana; >g7573480\| | | [Mycobacterium |
| | | | | emb\|CAB87839.1\| | | tuberculosis] |
| | | | | (AL163832) | | |
| | | | | putative protein | | |
| | | | | [Arabidopsis thaliana] | | |
| | | | | F27K19.3 putative | | |
| | | | | protein [ ] | | |
| 21 | XCCU3_839 | 121-3627 | 74 | K15N18.7 | 3.00E−52 | g7379447 (AL162754) |
| | | | | | | hypothetical protein |
| | | | | | | NMA0724 |
| | | | | | | [Neisseria |
| | | | | | | meningitidis] |
| 22 | XCCU3_1085 | 121-588 | 75 | MDH9.9 | 1.00E−19 | g5689489 (AB028999) |
| | | | | | | KIAA1076 protein |
| | | | | | | [Homo sapiens] |
| 23 | XCCU3_1139 | 121-1188 | 76 | MRI1.6 | 1.00E−41 | |
| 24 | XCCU3_1185 | 121-1938 | 77 | K13P22.5 | 2.00E−11 | g2896133 (AF047014) |
| | | | | | | outer membrane |
| | | | | | | esterase |
| | | | | | | [Salmonella |
| | | | | | | typhimurium] |
| 25 | XCCU3_1220 | 121-729 | 78 | g399091 | 1.00E−49 | g7212770 (AF044912) |
| | | | | PYROPHOSPHATE- | | H+ translocating |
| | | | | ENERGIZED VACUOLAR | | pyrophosphate |
| | | | | MEMBRANE PROTON | | synthase |
| | | | | PUMP | | [Rhodospirillum |
| | | | | (PYROPHOSPHATE- | | rubrum] |
| | | | | ENERGIZED INORGANIC | | |
| | | | | PYROPHOSPHATASE) | | |
| | | | | (H +− PPASE) | | |
| | | | | [Arabidopsis thaliana] | | |
| 26 | XCCU3_1221 | 121-1818 | 79 | g7024455 (AB034696) | 1.00E−108 | g7212770 (AF044912) |
| | | | | vacuolar- | | H+ translocating |
| | | | | pyrophosphatase | | pyrophosphate |
| | | | | like protein | | synthase |
| | | | | [Arabidopsis thaliana] | | [Rhodospirillum |
| | | | | | | rubrum] |
| 27 | XCCU3_1242 | 121-837 | 80 | g6403490 (AC010871) | 1.00E−15 | g7226826 (AE002508) |
| | | | | putative SCO1 protein | | conserved |
| | | | | [Arabidopsis thaliana] | | hypothetical protein |
| | | | | @(AC010871) | | [Neisseria |
| | | | | sg6403490\|gb\| | | meningitidis] |
| | | | | AAF07830.1\| | | |
| | | | | AC010871_6 sputative | | |
| | | | | SCO1 protein | | |
| | | | | [Arabidopsis thaliana] | | |
| | | | | T16O11.9 [ ] | | |
| 28 | XCCU3_1255 | 121-1341 | 81 | | | g7649650 (AL353864) |
| | | | | | | putative |
| | | | | | | oxidoreductase. |
| | | | | | | [Streptomyces |
| | | | | | | coelicolor A3(2)] |
| 29 | XCCU3_1953 | 121-807 | 82 | g6729496 (AL132966) | 4.00E−31 | g1169649 |
| | | | | putative protein | | HYPOTHETICAL 21.1 KD |
| | | | | [Arabidopsis thaliana] | | PROTEIN IN FASCIATION |
| | | | | @hypothetical | | LOCUS (ORF6) |
| | | | | sT45885 sprotein | | [Rhodococcus |
| | | | | F4P12.150 - Arabidopsis | | fascians] |
| | | | | thaliana; >g6729496\| | | |
| | | | | emb\|CAB67652.1\| | | |
| | | | | (AL132966) | | |
| | | | | putative protein | | |
| | | | | [Arabidopsis thaliana] | | |
| | | | | F4P12.15 putative | | |
| | | | | protein [ ] | | |
| 30 | XCCU3_2013 | 121-720 | 83 | g4567200 (AC007168) | 3.00E−19 | g4567200 (AC007168) |
| | | | | hypothetical protein | | hypothetical protein |
| | | | | [Arabidopsis thaliana] | | [Arabidopsis |
| | | | | @(AC007168) | | thaliana] |
| | | | | sg4567200\|gb\| | | |
| | | | | AAD23616.1\| | | |
| | | | | AC007168_7 | | |
| | | | | shypothetical | | |
| | | | | protein | | |
| | | | | [Arabidopsis thaliana] | | |
| | | | | AC007168.12 | | |
| | | | | At2g22260 [ ] | | |
| 31 | XCCU3_2157 | 121-591 | 84 | g3810594 (AC005398) | 2.00E−10 | g7471496 |
| | | | | hypothetical protein | | conserved |
| | | | | [Arabidopsis thaliana] | | hypothetical |
| | | | | @(AC005398) | | protein - |
| | | | | sg3810594\|gb\| | | Deinococcus |
| | | | | AAC69376.1\| | | radiodurans |
| | | | | shypothetical | | (strain R1) |
| | | | | protein | | [Deinococcus |
| | | | | [Arabidopsis thaliana] | | radiodurans] |
| | | | | AC005398.10 | | |
| | | | | At2g14660 [ ] | | |
| 32 | XCCU3_2177 | 121-1125 | 85 | g7340654 (AL162506) | 5.00E−94 | “g7436609 |
| | | | | fructose-bisphosphate | | fructose-bisphosphate |
| | | | | aldolase-like protein | | aldolase (EC4.1.2.13), |
| | | | | [Arabidopsis thaliana] | | cytosolic - common |
| | | | | @fructose-bisphosphate | | ice plant |
| | | | | sT48396 saldolase-like | | [Mesembryanthemum |
| | | | | protein - Arabidopsis | | crystallinum]” |
| | | | | thaliana; >g7340654\| | | |
| | | | | emb\|CAB82934.1\| | | |
| | | | | (AL162506) | | |
| | | | | fructose-bisphosphate | | |
| | | | | aldolase-like protein | | |
| | | | | [Arabidopsis thaliana] | | |
| | | | | F17C15.11 fructose- | | |
| | | | | bisphosphate aldolase- | | |
| | | | | like protein [ ] | | |
| 33 | XCCU3_2194 | 121-3585 | 86 | g2342683 (AC000106) | 1.00E−57 | g2342683 (AC000106) |
| | | | | Contains similarity to | | Contains similarity |
| | | | | Bos beta-mannosidase | | to Bos beta- |
| | | | | (gb\|U46067). | | mannosidase |
| | | | | [Arabidopsis thaliana] | | (gb\|U46067). |
| | | | | @(AC000106) | | [Arabidopsis |
| | | | | sg2342683\|gb\| | | thaliana] |
| | | | | AAB70407.1\| | | |
| | | | | s Contains similarity to | | |
| | | | | Bos beta-mannosidase | | |
| | | | | (gb\|U46067). | | |
| | | | | [Arabidopsis thaliana] | | |
| | | | | F7G19.12 [ ] | | |
| 34 | XCCU3_2384 | 121-1128 | 87 | g7486179 | 3.00E−63 | g7486179 |
| | | | | hypothetical protein | | hypothetical protein |
| | | | | F26B6.4 - | | F26B6.4 - |
| | | | | Arabidopsis thaliana | | Arabidopsis thaliana |
| | | | | [ ] | | [Arabidopsis |
| | | | | | | thaliana] |
| 35 | XCCU3_2394 | 121-876 | 88 | g8778719 (AC005106) | 2.00E−27 | g4105683 (AF049892) |
| | | | | T25N20.16 | | unknown |
| | | | | [Arabidopsis thaliana] | | [Oryza sativa |
| | | | | T25N20.15 [ ] | | subsp. indica] |
| 36 | XCCU3_2424 | 121-2028 | 89 | MIG10.8 | 9.00E−19 | g7518066 |
| | | | | | | hypothetical protein |
| | | | | | | PAB1246 - Pyrococcus |
| | | | | | | abyssi (strain Orsay) |
| | | | | | | [Pyrococcus abyssi] |
| 37 | XCCU3_2826 | 121-1017 | 90 | MSJ11.25 | 3.00E−48 | g4099843 (U90417) |
| | | | | | | delta 9 acyl-lipid |
| | | | | | | fatty acid desaturase |
| | | | | | | [Synechococcus |
| | | | | | | vulcanus] |
| 38 | XCCU3_2827 | 121-1371 | 91 | MEE5.5 | 8.00E−55 | g7477963 |
| | | | | | | probable dehydrogenase - |
| | | | | | | Mycobacterium |
| | | | | | | tuberculosis |
| | | | | | | (strain H37RV) |
| | | | | | | [Mycobacterium |
| | | | | | | tuberculosis] |
| 39 | XCCU3_2831 | 121-903 | 92 | g8671769 (AC069551) | 4.00E−19 | g7476359 |
| | | | | T10O22.15 | | hypothetical protein |
| | | | | [Arabidopsis thaliana] | | Rv0446c - |
| | | | | | | Mycobacterium |
| | | | | | | tuberculosis |
| | | | | | | (strain H37RV) |
| | | | | | | [Mycobacterium |
| | | | | | | tuberculosis] |
| 40 | XCCU3_2919 | 121-912 | 93 | g7431087 | 6.00E−10 | g7473368 |
| | | | | hypothetical protein | | probable 3-alpha- |
| | | | | F14M4.3 - | | hydroxysteroid |
| | | | | Arabidopsis thaliana | | dehydrogenase - |
| | | | | [ ] | | Deinococcus |
| | | | | | | radiodurans |
| | | | | | | (strain R1) |
| | | | | | | [Deinococcus |
| | | | | | | radiodurans] |
| 41 | XCCU3_3042 | 121-933 | 94 | g6715723 (AC016447) | 4.00E−26 | g5672692 (AB028448) |
| | | | | putative bifunctional | | nuclease I |
| | | | | nuclease | | [Hordeum vulgare] |
| | | | | [Arabidopsis thaliana] | | |
| | | | | @(AC016447) | | |
| | | | | sg6715723\|gb\| | | |
| | | | | AAF26484.1\| | | |
| | | | | AC016447_7 | | |
| | | | | sputative bifunctional | | |
| | | | | nuclease | | |
| | | | | [Arabidopsis thaliana] | | |
| | | | | T22E19.8 [ ] | | |
| 42 | XCCU3_3228 | 121-2880 | 95 | g1589778 (U62135) | 8.00E−10 | g6580650 (AL133469) |
| | | | | SPINDLY | | putative |
| | | | | [Arabidopsis thaliana] | | oligosaccharide |
| | | | | [ ] | | deacetylase |
| | | | | | | [Streptomyces |
| | | | | | | coelicolor A3(2)] |
| 43 | XCCU3_3277 | 121-2091 | 96 | K16H17.9 | 5.00E−11 | g7518601 |
| | | | | | | hypothetical protein |
| | | | | | | PH0361 - |
| | | | | | | Pyrococcus horikoshii |
| | | | | | | [Pyrococcus |
| | | | | | | horikoshii] |
| 44 | XCCU3_3380 | 121-936 | 97 | “g4263517 (AC004044) | 7.00E−23 | “g6630683 |
| | | | | similar to PHZF, | | (AP000969) |
| | | | | catalyzing the | | EST C25991(C11435) |
| | | | | hydroxylation of | | corresponds to a |
| | | | | phenazine-1-carboxylic | | region of the |
| | | | | acid to 2-hydroxy- | | predicted gene.; |
| | | | | phenazine-1-carboxylic | | Similar to |
| | | | | acid | | similar to PHZF, |
| | | | | [Arabidopsis thaliana] | | catalyzing the |
| | | | | [ ]” | | hydroxylation of |
| | | | | | | phenazine-1-carboxylic |
| | | | | | | acid to 2-hydroxy- |
| | | | | | | phenazine-1-carboxylic |
| | | | | | | acid. |
| | | | | | | (AC004044) |
| | | | | | | [Oryza sativa]” |
| 45 | XCCU3_3475 | 121-1404 | 98 | g6899923 (AL138651) | 2.00E−30 | g116093 |
| | | | | putative protein | | ISOPENICILLIN |
| | | | | [Arabidopsis thaliana] | | N EPIMERASE |
| | | | | @hypothetical | | [Streptomyces |
| | | | | sT48005 sprotein | | clavuligerus] |
| | | | | T17J13.90 - Arabidopsis | | |
| | | | | thaliana; >g6899923\| | | |
| | | | | emb\|CAB71873.1\| | | |
| | | | | (AL138651) putative | | |
| | | | | protein | | |
| | | | | [Arabidopsis thaliana] | | |
| | | | | T17J13.90 [ ] | | |
| 46 | XCCU3_3702 | 121-1023 | 99 | g5903052 (AC008016) | 1.00E−16 | g3114657 (AF060871) |
| | | | | Contains PF\|00561 | | haloalkane |
| | | | | alpha/beta hydrolase | | dehalogenase |
| | | | | fold. | | [Pseudomonas |
| | | | | [Arabidopsis thaliana] | | pavonaceae] |
| | | | | @(AC008016) | | |
| | | | | sg5903052\|gb\| | | |
| | | | | AAD55611.1\| | | |
| | | | | AC008016_21 | | |
| | | | | s Contains PF\|00561 | | |
| | | | | alpha/beta hydrolase | | |
| | | | | fold. | | |
| | | | | [Arabidopsis thaliana] | | |
| | | | | F6D8.27 [ ] | | |
| 47 | XCCU3_3860 | 121-1236 | 100 | g3334223 4- | 2.00E−21 | “g123491 |
| | | | | HYDROXYPHENYL | | 4-HYDROXYPHENYL |
| | | | | PYRUVATE | | PYRUVATE DIOXYGENASE |
| | | | | DIOXYGENASE (4HPPD) | | (4HPPD) (HPD) |
| | | | | (HPD) | | [Pseudomonas, |
| | | | | [Arabidopsis thaliana] | | P.J. 874, Peptide, |
| | | | | | | 357 aa]” |
| 48 | XCCU3_3861 | 121-1425 | 101 | “g7108615 (AF130845) | 1.00E−129 | “g7480748 |
| | | | | homogentisate | | probable |
| | | | | 1,2-dioxygenase | | homogentisate 1,2- |
| | | | | [Arabidopsis thaliana] | | dioxygenase |
| | | | | [ ]” | | Streptomyces |
| | | | | | | coelicolor |
| | | | | | | [Streptomyces |
| | | | | | | coelicolor A3 (2)]” |
| 49 | XCCU3_4058 | 121-1017 | 102 | g7649363 (AL353871) | 3.00E−15 | g6321804 SH3 |
| | | | | putative protein | | domain in C-terminus; |
| | | | | [Arabidopsis thaliana] | | Ysc84p |
| | | | | @hypothetical | | [Saccharomyces |
| | | | | sT49237 sprotein | | cerevisiae] |
| | | | | F7K15.80 - Arabidopsis | | |
| | | | | thaliana; >g7649363\| | | |
| | | | | emb\|CAB89044.1\| | | |
| | | | | (AL353871) putative | | |
| | | | | protein | | |
| | | | | [Arabidopsis thaliana] | | |
| | | | | F7K15.8 | | |
| | | | | F7K15_80 [ ] | | |
| 50 | XCCU3_4082 | 121-1179 | 103 | g7327825 (AL161946) | 1.00E−13 | g7298020 (AE003639) |
| | | | | putative protein | | CG9426 gene product |
| | | | | [Arabidopsis thaliana] | | [Drosophila |
| | | | | @hypothetical | | melanogaster] |
| | | | | sT48187 sprotein | | |
| | | | | F7A7.180 - Arabidopsis | | |
| | | | | thaliana; >g7327825\| | | |
| | | | | emb\|CAB82282.1\| | | |
| | | | | (AL161946) putative | | |
| | | | | protein | | |
| | | | | [Arabidopsis thaliana] | | |
| | | | | F7A7.18 putative | | |
| | | | | protein [ ] | | |
| 51 | XCCU3_4581 | 77-988 | 104 | g7487331 hypothetical | 2.00E−56 | g7487331 hypothetical |
| | | | | protein T22A6.50 - | | protein |
| | | | | Arabidopsis thaliana | | T22A6.50 - |
| | | | | [ ] | | Arabidopsis thaliana |
| | | | | | | [Arabidopsis |
| | | | | | | thaliana] |
| 52 | XCCU3_4884 | 121-948 | 105 | g4803960 (AC006202) | 2.00E−33 | g5360551 (AB022175) |
| | | | | putative carbonic | | a-type carbonic |
| | | | | anhydrase | | anhydrase |
| | | | | [Arabidopsis thaliana] | | [Rhodopseudomonas |
| | | | | @(AC006202) | | palustris] |
| | | | | sg4803960\|gb\| | | |
| | | | | AAD29832.1\| | | |
| | | | | AC006202_10 | | |
| | | | | sputative carbonic | | |
| | | | | anhydrase | | |
| | | | | [Arabidopsis thaliana] | | |
| | | | | AC006202.3 | | |
| | | | | At2g28210 [ ] | | |
| 53 | XCCU3_5000 | 102-2456 | 106 | g6175185 (AC011437) | 3.00E−14 | g4803678 (X56958) |
| | | | | ankyrin-like protein | | ankyrin (brank-2) |
| | | | | [Arabidopsis thaliana] | | [Homo sapiens] |
| | | | | @(AC011437) | | |
| | | | | sg6175185\|gb\| | | |
| | | | | AAF04911.1\| | | |
| | | | | AC011437_26 | | |
| | | | | sankyrin-like protein | | |
| | | | | [Arabidopsis thaliana] | | |
| | | | | F7O18.18 [ ] | | |

| Seq | NRAA_{—} | | MAIZE_{—} | | SOY_{—} | | RICE_{—} |
|---|---|---|---|---|---|---|---|
| Num | TOP_EVAL | MAIZE_TOP | TOP_EVAL | SOY_TOP | TOP_EVAL | RICE_TOP | TOP_EVAL |
| 1 | 4.00E−39 | | | 6916_{—} | 1.00E−40 | | |
| | | | | 1.R1040277 | | | |
| 2 | 2.00E−35 | | | | | OJ00 | 3.00E−12 |
| | | | | | | 0119_{—} | |
| | | | | | | 04.0208.C2 | |
| 3 | 2.00E−42 | ZEAMA-23MAY00- | 7.00E−38 | | | | |
| | | CLUSTER22290_1 | | | | | |
| 4 | 2.00E−17 | ZEAMA-23MAY00- | 2.00E−14 | | | OJ00 | 1.00E−16 |
| | | CLUSTER5260_1 | | | | 0314_{—} | |
| | | | | | | 11.0421.C11 | |
| 5 | 9.00E−19 | ZEAMA-23MAY00- | 1.00E−27 | | | | |
| | | CLUSTER14060_1 | | | | | |
| 6 | 3.00E−35 | ZEAMA-23MAY00- | 1.00E−35 | 3329_{—} | 3.00E−33 | | |
| | | CLUSTER1955_1 | | 1.R1040 | | | |
| 7 | 1.00E−14 | ZEAMA-23MAY00- | 2.00E−11 | 290_{—} | 4.00E−11 | | |
| | | CLUSTER261990_1 | | 1.R1040 | | | |
| 8 | 2.00E−21 | | | | | | |
| 9 | 1.00E−23 | ZEAMA-23MAY00- | 5.00E−20 | 6796_{—} | 8.00E−21 | | |
| | | CLUSTER12838_2 | | 1.R1040 | | | |
| 10 | | ZEAMA-23MAY00- | 2.00E−22 | | | | |
| | | CLUSTER12990_1 | | | | | |
| 11 | 8.00E−27 | ZEAMA-23MAY00- | 3.00E−24 | 21108_{—} | 4.00E−12 | | |
| | | CLUSTER56970_1271 | | 1.R1040 | | | |
| 12 | 1.00E−110 | ZEAMA-23MAY00- | 6.00E−17 | | | | |
| | | CLUSTER435029_1 | | | | | |
| 13 | 6.00E−91 | ZEAMA-23MAY00- | 1.00E−38 | 155874_{—} | 2.00E−25 | OJ00 | 1.00E−11 |
| | | CLUSTER8031_2 | | 1.R1040 | | 0320_{—} | |
| | | | | | | 24.0411.C7 | |
| 14 | 1.00E−30 | | | | | | |
| 15 | 4.00E−47 | ZEAMA-23MAY00- | 3.00E−16 | 25536_{—} | 2.00E−28 | | |
| | | CLUSTER90594_1 | | 1.R1040 | | | |
| 16 | 1.00E−40 | ZEAMA-23MAY00- | 1.00E−31 | 3329_{—} | 4.00E−31 | | |
| | | CLUSTER1955_1 | | 1.R1040 | | | |
| 17 | 3.00E−73 | ZEAMA-23MAY00- | 1.00E−12 | 163147_{—} | 3.00E−12 | | |
| | | CLUSTER84325_1 | | 1.R1040 | | | |
| 18 | 1.00E−30 | ZEAMA-23MAY00- | 5.00E−32 | 14964_{—} | 4.00E−33 | OJ99 | 1.00E−20 |
| | | CLUSTER523510_1 | | 1.R1040 | | 0923_{—} | |
| | | | | | | 09.9C23.C30 | |
| 19 | 5.00E−54 | ZEAMA-23MAY00- | 1.00E−31 | | | | |
| | | CLUSTER3818_1 | | | | | |
| 20 | 8.00E−29 | | | | | | |
| 21 | 1.00E−167 | ZEAMA-23MAY00- | 1.00E−26 | | | OJ00 | 1.00E−12 |
| | | CLUSTER20204_1 | | | | 0210_{—} | |
| | | | | | | 09.0419.C2 | |
| 22 | 3.00E−18 | ZEAMA-23MAY00- | 9.00E−23 | 75558_{—} | 2.00E−16 | OJ00 | 1.00E−11 |
| | | CLUSTER112212_1 | | 1.R1040 | | 0302_{—} | |
| | | | | | | 02.0419.C20 | |
| 23 | | | | | | | |
| 24 | 7.00E−49 | ZEAMA-23MAY00- | 2.00E−12 | | | | |
| | | CLUSTER183755_1 | | | | | |
| 25 | 8.00E−76 | ZEAMA-23MAY00- | 3.00E−45 | 1572_{—} | 4.00E−49 | OJ99 | 1.00E−46 |
| | | CLUSTER10681_1 | | 2.R1040 | | 0803_{—} | |
| | | | | | | 12.0103.C2 | |
| 26 | 1.00E−157 | ZEAMA-23MAY00- | 2.00E−70 | | | OJ00 | 1.00E−65 |
| | | CLUSTER500_11 | | | | 0350_{—} | |
| | | | | | | 03.0310.C10 | |
| 27 | 1.00E−24 | ZEAMA-23MAY00- | 1.00E−17 | 22489_{—} | 5.00E−11 | | |
| | | CLUSTER73482_1 | | 1.R1040 | | | |
| 28 | 5.00E−15 | ZEAMA-23MAY00- | 6.00E−61 | | | | |
| | | CLUSTER253481_1 | | | | | |
| 29 | 3.00E−33 | ZEAMA-23MAY00- | 1.00E−31 | | | OJ00 | 4.00E−30 |
| | | CLUSTER27935_1 | | | | 0313_{—} | |
| | | | | | | 23.0419.C26 | |
| 30 | 4.00E−18 | | | | | | |
| 31 | 1.00E−38 | | | | | OJ00 | 5.00E−10 |
| | | | | | | 0112_{—} | |
| | | | | | | 06.0426.C11 | |
| 32 | 1.00E−93 | | | | | | |
| 33 | 1.00E−56 | ZEAMA-23MAY00- | 1.00E−30 | 46430_{—} | 1.00E−25 | | |
| | | CLUSTER19232_1 | | 1.R1040 | | | |
| 34 | 4.00E−62 | ZEAMA-23MAY00- | 1.00E−20 | 77529_{—} | 5.00E−12 | | |
| | | CLUSTER1131_25 | | 1.R1040 | | | |
| 35 | 1.00E−27 | ZEAMA-23MAY00- | 2.00E−28 | 24650_{—} | 3.00E−26 | OJ99 | 2.00E−25 |
| | | CLUSTER94488_1 | | 1.R1040 | | 0605_{—} | |
| | | | | | | 41.0225.C3 | |
| 36 | 6.00E−23 | ZEAMA-23MAY00- | 1.00E−18 | 237931_{—} | 1.00E−12 | | |
| | | CLUSTER4785_1 | | 1.R1040 | | | |
| 37 | 1.00E−51 | | | 165186_{—} | 4.00E−14 | | |
| | | | | 1.R1040 | | | |
| 38 | 1.00E−79 | | | | | | |
| 39 | 2.00E−25 | ZEAMA-23MAY00- | 9.00E−27 | 72546_{—} | 1.00E−11 | OJ99 | 1.00E−11 |
| | | CLUSTER4781_1 | | 1.R1040 | | 0429_{—} | |
| | | | | | | 03.9B02.C46 | |
| 40 | 2.00E−25 | ZEAMA-23MAY00- | 2.00E−13 | 119588_{—} | 3.00E−10 | OJ00 | 4.00E−14 |
| | | CLUSTER13977_2 | | 1.R1040 | | 0209_{—} | |
| | | | | | | 21.0313.C17 | |
| 41 | 1.00E−25 | ZEAMA-23MAY00- | 4.00E−23 | | | | |
| | | CLUSTER1856_3 | | | | | |
| 42 | 1.00E−21 | | | | | | |
| 43 | 1.00E−150 | | | | | | |
| 44 | 8.00E−25 | ZEAMA-23MAY00- | 3.00E−27 | | | | |
| | | CLUSTER45498_1 | | | | | |
| 45 | 2.00E−41 | ZEAMA-23MAY00- | 2.00E−19 | 79185_{—} | 5.00E−14 | OJ99 | 3.00E−38 |
| | | CLUSTER12860_1 | | 1.R10.40 | | 0612_{—} | |
| | | | | | | 30.9B05.C10 | |
| 46 | 6.00E−27 | ZEAMA23MAY00- | 2.00E−12 | 72100_{—} | 2.00E−14 | OJ99 | 2.00E−11 |
| | | CLUSTER1922_1 | | 1.R1040 | | 0915_{—} | |
| | | | | | | 11.0103.C23 | |
| 47 | 1.00E−102 | ZEAMA-23MAY00- | 8.00E−12 | | | | |
| | | CLUSTER27415_1 | | | | | |
| 48 | 1.00E−150 | ZEAMA-23MAY00- | 1.00E−71 | | | OJ99 | 2.00E−59 |
| | | CLUSTER1031_1 | | | | 1201_{—} | |
| | | | | | | 14.0118.C4 | |
| 49 | 2.00E−22 | ZEAMA-23MAY00- | 2.00E−15 | 24743_{—} | 1.00E−14 | | |
| | | CLUSTER32737_1 | | 1.R1040 | | | |
| 50 | 2.00E−20 | ZEAMA-23MAY00- | 3.00E−13 | | | OJ99 | 4.00E−11 |
| | | CLUSTER107757_1 | | | | 0810_{—} | |
| | | | | | | 13.9C23.C29 | |
| 51 | 2.00E−55 | ZEAMA-23MAY00- | 2.00E−23 | | | OJ00 | 1.00E−40 |
| | | CLUSTER394210_1 | | | | 0327_{—} | |
| | | | | | | 29.0419.C13 | |
| 52 | 2.00E−53 | ZEAMA-23MAY00- | 2.00E−28 | uC-gmro | 2.00E−12 | OJ00 | 1.00E−29 |
| | | CLUSTER79822_1 | | pic0 | | 0313_{—} | |
| | | | | 50b1 | | 36.0421.C7 | |
| | | | | 0b1 | | | |
| 53 | 1.00E−47 | ZEAMA-23MAY00- | 2.00E−13 | 290_{—} | 5.00E−10 | OJ00 | 7.00E−11 |
| | | CLUSTER797_1 | | 1.R1040 | | 0306_{—} | |
| | | | | | | 20.0417.C9 | |

Table Headings:
“Seq Num” is the SEQ ID NO of the sequence in the sequence listing.
“Seq ID” is a name assigned to the sequence. Names beginning with AGR are from
Agrobacterium tumefaciens and those beginning with XCCU are from Xanthomonas campestris pv. campestris.
“Coding Seq” refers to the coding coordinates within the gene which are translated to a protein
“Pep Num” is the SEQ ID NO of the protein in the sequence listing
“ATHAL_TOP” is a public annotation provided for the top BLAST hit for the sequence from the Arabidopsis thaliana genome and may include a gi number or GenBank identifier.
“ATHAL_TOP_EVAL” is the E value of the top hit in the Arabidopsis thaliana protein database using BLASTP version 2.0.8 [Jan 5, 1999] and default parameters.
“NRAA_TOP” is a public annotation provided for the top BLAST hit for the sequence from the non-redundant amino acid database and may include a gi number or GenBank identifier.
“NRAA_TOP_EVAL” is the E value of the top hit in the non-redundant amino acid database using BLASTP version 2.0.8 [Jan 5, 1999] and default parameters.
“MAIZE_TOP” is an arbitrary identifier of unpublished sequence.
“MAIZE_TOP_EVAL” is the E value of the top hit in the unpublished nucleic acid database using TBLASTN version 2.0.8 [Jan 5, 1999] and default parameters.
“SOY_TOP” is an arbitrary identifier of unpublished sequence.
“SOY_TOP_EVAL” is the E value of the top hit in the unpublished nucleic acid database using TBLASTN version 2.0.8 [Jan 5, 1999] and default parameters.
“RICE_TOP” is an arbitrary identifier of sequence available on Monsanto's web site.
“RICE_TOP_EVAL” is the E value of the top hit in Monsanto's rice nucleic acid database using TBLASTN version 2.0.8 [Jan 5, 1999] and default parameters.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. AR such similar substitutes and modifications apparent to those skilled in the art are deemed to be within the spirit, scope and concept of the invention as defined by the appended claims.

All publications and patent applications cited herein are incorporated by reference in their entirely to the same extent as if each individual publication or patent application was specifically and individually indicated to be incorporated by reference.

1-12. (canceled)

13. A candidate set of microbial genes derived using the method of claim 1.

14. A candidate subset of microbial genes derived using the method of claim 3.

15. A candidate set of claim 13 comprising one or more of the nucleic acid sequences selected from the group consisting of SEQ ID NO: 1 through SEQ ID NO: 53.

16. A candidate set of claim 13 comprising one or more nucleic acid molecules encoding proteins having a sequence selected from the group consisting of SEQ ID NO: 54 through SEQ ID NO: 106.

17. A transformed plant comprising at least one microbial gene selected from the group consisting of SEQ ID NO: 1 through SEQ ID NO: 53.

18. A transformed plant comprising at least one microbial gene selected from the group consisting of:
a. a nucleic acid molecule encoding a protein having an amino acid sequence of SEQ ID NO: 54 through SEQ ID NO: 106;
b. a nucleic acid molecule which encodes a functional homolog of a protein having a sequence of SEQ ID NO: 54 through SEQ ID NO: 106; and
c. a nucleic acid molecule encoding a polypeptide of at least 20 amino acid residues which are identical to a 20 amino acid segment in a protein having a sequence of SEQ ID NO: 54 through SEQ ID NO: 106, and which is a functional homolog of said protein.

19. A method of screening non-plant gene sequences for sequences predicted to be useful in improving crops, comprising:
(a) selecting a crop plant and a non-plant subject organism;
(b) comparing gene sequences of said crop plant and gene sequences of said non-plant subject organism;
(c) selecting a candidate set of gene sequences comprising gene sequences of said non-plant subject organism that are similar to gene sequences of said crop plant and are predicted to be useful in improving crops.

20. The method of claim 19, wherein said candidate set comprises gene sequences of said non-plant subject organism with a BLAST E value greater than or equal to about E-9 when compared to said gene sequences of said crop plant.

21. The method of claim 19, further comprising the step of selecting a candidate subset from said candidate set.

22. The method of claim 19, further comprising the step of selecting a candidate subset from said candidate set by subtracting gene sequences homologous to those of a reference organism from said gene sequences of said non-plant subject organism.

23. The method of claim 19, wherein said reference organism comprises an organism more closely related to said non-plant subject organism than to said crop plant.

24. A transgenic plant, containing in its genome a transgene selected from a candidate set of gene sequences, said candidate set comprising gene sequences of a non-plant subject organism selected for their similarity to gene sequences of said plant.
